SEQUENCE LISTING (429 sequences)

SEQ ID NO: 1 - protein, 5 aa, Artificial Sequence
  Cyclic peptide with His-Ala-Val cell adhesion recognition motif
CHAVC

SEQ ID NO: 2 - protein, 6 aa, Artificial Sequence
  Cyclic peptide with His-Ala-Val cell adhesion recognition motif
CHAVCY

SEQ ID NO: 3 - protein, 6 aa, Artificial Sequence
  Cyclic peptide with His-Ala-Val cell adhesion recognition motif
CHAVDC

SEQ ID NO: 4 - protein, 7 aa, Artificial Sequence
  Cyclic peptide with His-Ala-Val cell adhesion recognition motif
CHAVDIC

SEQ ID NO: 5 - protein, 8 aa, Artificial Sequence
  Cyclic peptide with His-Ala-Val cell adhesion recognition motif
CHAVDINC

SEQ ID NO: 6 - protein, 9 aa, Artificial Sequence
  Cyclic peptide with His-Ala-Val cell adhesion recognition motif
CHAVDINGC

SEQ ID NO: 7 - protein, 6 aa, Artificial Sequence
  Cyclic peptide with His-Ala-Val cell adhesion recognition motif
CAHAVC

SEQ ID NO: 8 - protein, 7 aa, Artificial Sequence
  Cyclic peptide with His-Ala-Val cell adhesion recognition motif
CAHAVDC

SEQ ID NO: 9 - protein, 8 aa, Artificial Sequence
  Cyclic peptide with His-Ala-Val cell adhesion recognition motif
CAHAVDIC

SEQ ID NO: 10 - protein, 8 aa, Artificial Sequence
  Cyclic peptide with His-Ala-Val cell adhesion recognition motif
CRAHAVDC

SEQ ID NO: 11 - protein, 8 aa, Artificial Sequence
  Cyclic peptide with His-Ala-Val cell adhesion recognition motif
CLRAHAVC

SEQ ID NO: 12 - protein, 9 aa, Artificial Sequence
  Cyclic peptide with His-Ala-Val cell adhesion recognition motif
CLRAHAVDC

SEQ ID NO: 13 - protein, 6 aa, Artificial Sequence
  Cyclic peptide with His-Ala-Val cell adhesion recognition motif
CSHAVC

SEQ ID NO: 14 - protein, 7 aa, Artificial Sequence
  Cyclic peptide with His-Ala-Val cell adhesion recognition motif
CFSHAVC

SEQ ID NO: 15 - protein, 8 aa, Artificial Sequence
  Cyclic peptide with His-Ala-Val cell adhesion recognition motif
CLFSHAVC

SEQ ID NO: 16 - protein, 6 aa, Artificial Sequence
  Cyclic peptide with His-Ala-Val cell adhesion recognition motif
CHAVSC

SEQ ID NO: 17 - protein, 7 aa, Artificial Sequence
  Cyclic peptide with His-Ala-Val cell adhesion recognition motif
CSHAVSC

SEQ ID NO: 18 - protein, 8 aa, Artificial Sequence
  Cyclic peptide with His-Ala-Val cell adhesion recognition motif
CSHAVSSC

SEQ ID NO: 19 - protein, 7 aa, Artificial Sequence
  Cyclic peptide with His-Ala-Val cell adhesion recognition motif
CHAVSSC

SEQ ID NO: 20 - protein, 5 aa, Artificial Sequence
  Cyclic peptide with His-Ala-Val cell adhesion recognition motif
KHAVD

SEQ ID NO: 21 - protein, 5 aa, Artificial Sequence
  Cyclic peptide with His-Ala-Val cell adhesion recognition motif
DHAVK

SEQ ID NO: 22 - protein, 5 aa, Artificial Sequence
  Cyclic peptide with His-Ala-Val cell adhesion recognition motif
KHAVE

SEQ ID NO: 23 - protein, 6 aa, Artificial Sequence
  Cyclic peptide with His-Ala-Val cell adhesion recognition motif
AHAVDI

SEQ ID NO: 24 - protein, 7 aa, Artificial Sequence
  Cyclic peptide with His-Ala-Val cell adhesion recognition motif
SHAVDSS

SEQ ID NO: 25 - protein, 8 aa, Artificial Sequence
  Cyclic peptide with His-Ala-Val cell adhesion recognition motif
KSHAVSSD

SEQ ID NO: 26 - protein, 6 aa, Artificial Sequence
  Cyclic peptide with His-Ala-Val cell adhesion recognition motif
CHAVCS

SEQ ID NO: 27 - protein, 6 aa, Artificial Sequence
  Cyclic peptide with His-Ala-Val cell adhesion recognition motif
SCHAVC

SEQ ID NO: 28 - protein, 7 aa, Artificial Sequence
  Cyclic peptide with His-Ala-Val cell adhesion recognition motif
CHAVCSS

SEQ ID NO: 29 - protein, 7 aa, Artificial Sequence
  Cyclic peptide with His-Ala-Val cell adhesion recognition motif
SCHAVCS

SEQ ID NO: 30 - protein, 6 aa, Artificial Sequence
  Cyclic peptide with His-Ala-Val cell adhesion recognition motif
CHAVCT

SEQ ID NO: 31 - protein, 6 aa, Artificial Sequence
  Cyclic peptide with His-Ala-Val cell adhesion recognition motif
CHAVCE

SEQ ID NO: 32 - protein, 6 aa, Artificial Sequence
  Cyclic peptide with His-Ala-Val cell adhesion recognition motif
CHAVCD

SEQ ID NO: 33 - protein, 6 aa, Artificial Sequence
  Cyclic peptide with His-Ala-Val cell adhesion recognition motif
CHAVYC

SEQ ID NO: 34 - protein, 6 aa, Artificial Sequence
  Cyclic peptide with His-Ala-Val cell adhesion recognition motif
CHAVCY

SEQ ID NO: 35 - protein, 5 aa, Artificial Sequence
  Cyclic peptide with His-Ala-Val cell adhesion recognition motif
CHAVC

SEQ ID NO: 36 - protein, 5 aa, Artificial Sequence
  Cyclic peptide with His-Ala-Val cell adhesion recognition motif
CHAVC

SEQ ID NO: 37 - protein, 5 aa, Artificial Sequence
  Cyclic peptide with His-Ala-Val cell adhesion recognition motif
CHAVX

SEQ ID NO: 38 - protein, 5 aa, Artificial Sequence
  Cyclic peptide with His-Ala-Val cell adhesion recognition motif
XHAVC

SEQ ID NO: 39 - protein, 6 aa, Artificial Sequence
  Cyclic peptide with His-Ala-Val cell adhesion recognition motif
CHAVPC

SEQ ID NO: 40 - protein, 6 aa, Artificial Sequence
  Cadherin antagonist motif
XWVXXP

SEQ ID NO: 41 - protein, 4 aa, Artificial Sequence
  Cadherin antagonist containing Trp- cell adhesion recognition sequence
DWVI

SEQ ID NO: 42 - protein, 4 aa, Artificial Sequence
  Cadherin antagonist containing Trp- cell adhesion recognition sequence
DWVV

SEQ ID NO: 43 - protein, 4 aa, Artificial Sequence
  Cadherin antagonist containing Trp- cell adhesion recognition sequence
DWVM

SEQ ID NO: 44 - protein, 5 aa, Artificial Sequence
  Cadherin antagonist containing Trp- cell adhesion recognition sequence
DWVIP

SEQ ID NO: 45 - protein, 5 aa, Artificial Sequence
  Cadherin antagonist containing Trp- cell adhesion recognition sequence
DWVIA

SEQ ID NO: 46 - protein, 5 aa, Artificial Sequence
  Cadherin antagonist containing Trp- cell adhesion recognition sequence
DWVVP

SEQ ID NO: 47 - protein, 6 aa, Artificial Sequence
  Cadherin antagonist containing Trp- cell adhesion recognition sequence
DWVVPP

SEQ ID NO: 48 - protein, 6 aa, Artificial Sequence
  Cadherin antagonist containing Trp- cell adhesion recognition sequence
DWVVAP

SEQ ID NO: 49 - protein, 6 aa, Artificial Sequence
  Cadherin antagonist containing Trp- cell adhesion recognition sequence
DWVMPP

SEQ ID NO: 50 - protein, 6 aa, Artificial Sequence
  Cadherin antagonist containing Trp- cell adhesion recognition sequence
DWVMAP

SEQ ID NO: 51 - protein, 4 aa, Artificial Sequence
  Cadherin antagonist containing Trp- cell adhesion recognition sequence
EWVI

SEQ ID NO: 52 - protein, 4 aa, Artificial Sequence
  Cadherin antagonist containing Trp- cell adhesion recognition sequence
EWVV

SEQ ID NO: 53 - protein, 4 aa, Artificial Sequence
  Cadherin antagonist containing Trp- cell adhesion recognition sequence
EWVM

SEQ ID NO: 54 - protein, 5 aa, Artificial Sequence
  Cadherin antagonist containing Trp- cell adhesion recognition sequence
EWVIP

SEQ ID NO: 55 - protein, 5 aa, Artificial Sequence
  Cadherin antagonist containing Trp- cell adhesion recognition sequence
EWVIA

SEQ ID NO: 56 - protein, 5 aa, Artificial Sequence
  Cadherin antagonist containing Trp- cell adhesion recognition sequence
EWVVP

SEQ ID NO: 57 - protein, 6 aa, Artificial Sequence
  Cadherin antagonist containing Trp- cell adhesion recognition sequence
EWVVPP

SEQ ID NO: 58 - protein, 6 aa, Artificial Sequence
  Cadherin antagonist containing Trp- cell adhesion recognition sequence
EWVVAP

SEQ ID NO: 59 - protein, 6 aa, Artificial Sequence
  Cadherin antagonist containing Trp- cell adhesion recognition sequence
EWVMPP

SEQ ID NO: 60 - protein, 6 aa, Artificial Sequence
  Cadherin antagonist containing Trp- cell adhesion recognition sequence
EWVMAP

SEQ ID NO: 61 - protein, 4 aa, Artificial Sequence
  Cadherin antagonist containing Trp- cell adhesion recognition sequence
WVIP

SEQ ID NO: 62 - protein, 4 aa, Artificial Sequence
  Cadherin antagonist containing Trp- cell adhesion recognition sequence
WVIA

SEQ ID NO: 63 - protein, 4 aa, Artificial Sequence
  Cadherin antagonist containing Trp- cell adhesion recognition sequence
WVVP

SEQ ID NO: 64 - protein, 4 aa, Artificial Sequence
  Cadherin antagonist containing Trp- cell adhesion recognition sequence
WVVA

SEQ ID NO: 65 - protein, 4 aa, Artificial Sequence
  Cadherin antagonist containing Trp- cell adhesion recognition sequence
WVMP

SEQ ID NO: 66 - protein, 4 aa, Artificial Sequence
  Cadherin antagonist containing Trp- cell adhesion recognition sequence
WVMA

SEQ ID NO: 67 - protein, 5 aa, Artificial Sequence
  Cadherin antagonist containing Trp- cell adhesion recognition sequence
WVIPP

SEQ ID NO: 68 - protein, 5 aa, Artificial Sequence
  Cadherin antagonist containing Trp- cell adhesion recognition sequence
WVIAP

SEQ ID NO: 69 - protein, 5 aa, Artificial Sequence
  Cadherin antagonist containing Trp- cell adhesion recognition sequence
WVVPP

SEQ ID NO: 70 - protein, 5 aa, Artificial Sequence
  Cadherin antagonist containing Trp- cell adhesion recognition sequence
WVVAP

SEQ ID NO: 71 - protein, 5 aa, Artificial Sequence
  Cadherin antagonist containing Trp- cell adhesion recognition sequence
WVMPP

SEQ ID NO: 72 - protein, 5 aa, Artificial Sequence
  Cadherin antagonist containing Trp- cell adhesion recognition sequence
WVMAP

SEQ ID NO: 73 - protein, 4 aa, Artificial Sequence
  Cadherin antagonist containing Trp- cell adhesion recognition sequence
DWII

SEQ ID NO: 74 - protein, 4 aa, Artificial Sequence
  Cadherin antagonist containing Trp- cell adhesion recognition sequence
DWIV

SEQ ID NO: 75 - protein, 4 aa, Artificial Sequence
  Cadherin antagonist containing Trp- cell adhesion recognition sequence
DWIM

SEQ ID NO: 76 - protein, 5 aa, Artificial Sequence
  Cadherin antagonist containing Trp- cell adhesion recognition sequence
DWIIP

SEQ ID NO: 77 - protein, 5 aa, Artificial Sequence
  Cadherin antagonist containing Trp- cell adhesion recognition sequence
DWIIA

SEQ ID NO: 78 - protein, 5 aa, Artificial Sequence
  Cadherin antagonist containing Trp- cell adhesion recognition sequence
DWIVP

SEQ ID NO: 79 - protein, 6 aa, Artificial Sequence
  Cadherin antagonist containing Trp- cell adhesion recognition sequence
DWIVPP

SEQ ID NO: 80 - protein, 6 aa, Artificial Sequence
  Cadherin antagonist containing Trp- cell adhesion recognition sequence
DWIVAP

SEQ ID NO: 81 - protein, 6 aa, Artificial Sequence
  Cadherin antagonist containing Trp- cell adhesion recognition sequence
DWIMPP

SEQ ID NO: 82 - protein, 6 aa, Artificial Sequence
  Cadherin antagonist containing Trp- cell adhesion recognition sequence
DWIMAP

SEQ ID NO: 83 - protein, 4 aa, Artificial Sequence
  Cadherin antagonist containing Trp- cell adhesion recognition sequence
EWII

SEQ ID NO: 84 - protein, 4 aa, Artificial Sequence
  Cadherin antagonist containing Trp- cell adhesion recognition sequence
EWIV

SEQ ID NO: 85 - protein, 4 aa, Artificial Sequence
  Cadherin antagonist containing Trp- cell adhesion recognition sequence
EWIM

SEQ ID NO: 86 - protein, 5 aa, Artificial Sequence
  Cadherin antagonist containing Trp- cell adhesion recognition sequence
EWIIP

SEQ ID NO: 87 - protein, 5 aa, Artificial Sequence
  Cadherin antagonist containing Trp- cell adhesion recognition sequence
EWIIA

SEQ ID NO: 88 - protein, 5 aa, Artificial Sequence
  Cadherin antagonist containing Trp- cell adhesion recognition sequence
EWIVP

SEQ ID NO: 89 - protein, 6 aa, Artificial Sequence
  Cadherin antagonist containing Trp- cell adhesion recognition sequence
EWIVPP

SEQ ID NO: 90 - protein, 6 aa, Artificial Sequence
  Cadherin antagonist containing Trp- cell adhesion recognition sequence
EWIVAP

SEQ ID NO: 91 - protein, 6 aa, Artificial Sequence
  Cadherin antagonist containing Trp- cell adhesion recognition sequence
EWIMPP

SEQ ID NO: 92 - protein, 6 aa, Artificial Sequence
  Cadherin antagonist containing Trp- cell adhesion recognition sequence
EWIMAP

SEQ ID NO: 93 - protein, 4 aa, Artificial Sequence
  Cadherin antagonist containing Trp- cell adhesion recognition sequence
WIIP

SEQ ID NO: 94 - protein, 4 aa, Artificial Sequence
  Cadherin antagonist containing Trp- cell adhesion recognition sequence
WIIA

SEQ ID NO: 95 - protein, 4 aa, Artificial Sequence
  Cadherin antagonist containing Trp- cell adhesion recognition sequence
WIVP

SEQ ID NO: 96 - protein, 4 aa, Artificial Sequence
  Cadherin antagonist containing Trp- cell adhesion recognition sequence
WIVA

SEQ ID NO: 97 - protein, 4 aa, Artificial Sequence
  Cadherin antagonist containing Trp- cell adhesion recognition sequence
WIMP

SEQ ID NO: 98 - protein, 4 aa, Artificial Sequence
  Cadherin antagonist containing Trp- cell adhesion recognition sequence
WIMA

SEQ ID NO: 99 - protein, 5 aa, Artificial Sequence
  Cadherin antagonist containing Trp- cell adhesion recognition sequence
WIIPP

SEQ ID NO: 100 - protein, 5 aa, Artificial Sequence
  Cadherin antagonist containing Trp- cell adhesion recognition sequence
WIIAP

SEQ ID NO: 101 - protein, 5 aa, Artificial Sequence
  Cadherin antagonist containing Trp- cell adhesion recognition sequence
WIVPP

SEQ ID NO: 102 - protein, 5 aa, Artificial Sequence
  Cadherin antagonist containing Trp- cell adhesion recognition sequence
WIVAP

SEQ ID NO: 103 - protein, 5 aa, Artificial Sequence
  Cadherin antagonist containing Trp- cell adhesion recognition sequence
WIMPP

SEQ ID NO: 104 - protein, 5 aa, Artificial Sequence
  Cadherin antagonist containing Trp- cell adhesion recognition sequence
WIMAP

SEQ ID NO: 105 - protein, 4 aa, Artificial Sequence
  Cadherin antagonist containing Trp- cell adhesion recognition sequence
DWLI

SEQ ID NO: 106 - protein, 4 aa, Artificial Sequence
  Cadherin antagonist containing Trp- cell adhesion recognition sequence
DWLV

SEQ ID NO: 107 - protein, 4 aa, Artificial Sequence
  Cadherin antagonist containing Trp- cell adhesion recognition sequence
DWLM

SEQ ID NO: 108 - protein, 5 aa, Artificial Sequence
  Cadherin antagonist containing Trp- cell adhesion recognition sequence
DWLIP

SEQ ID NO: 109 - protein, 5 aa, Artificial Sequence
  Cadherin antagonist containing Trp- cell adhesion recognition sequence
DWLIA

SEQ ID NO: 110 - protein, 5 aa, Artificial Sequence
  Cadherin antagonist containing Trp- cell adhesion recognition sequence
DWLVP

SEQ ID NO: 111 - protein, 6 aa, Artificial Sequence
  Cadherin antagonist containing Trp- cell adhesion recognition sequence
DWLVPP

SEQ ID NO: 112 - protein, 6 aa, Artificial Sequence
  Cadherin antagonist containing Trp- cell adhesion recognition sequence
DWLVAP

SEQ ID NO: 113 - protein, 6 aa, Artificial Sequence
  Cadherin antagonist containing Trp- cell adhesion recognition sequence
DWLMPP

SEQ ID NO: 114 - protein, 6 aa, Artificial Sequence
  Cadherin antagonist containing Trp- cell adhesion recognition sequence
DWLMAP

SEQ ID NO: 115 - protein, 4 aa, Artificial Sequence
  Cadherin antagonist containing Trp- cell adhesion recognition sequence
EWLI

SEQ ID NO: 116 - protein, 4 aa, Artificial Sequence
  Cadherin antagonist containing Trp- cell adhesion recognition sequence
EWLV

SEQ ID NO: 117 - protein, 4 aa, Artificial Sequence
  Cadherin antagonist containing Trp- cell adhesion recognition sequence
EWLM

SEQ ID NO: 118 - protein, 5 aa, Artificial Sequence
  Cadherin antagonist containing Trp- cell adhesion recognition sequence
EWLIP

SEQ ID NO: 119 - protein, 5 aa, Artificial Sequence
  Cadherin antagonist containing Trp- cell adhesion recognition sequence
EWLIA

SEQ ID NO: 120 - protein, 5 aa, Artificial Sequence
  Cadherin antagonist containing Trp- cell adhesion recognition sequence
EWLVP

SEQ ID NO: 121 - protein, 6 aa, Artificial Sequence
  Cadherin antagonist containing Trp- cell adhesion recognition sequence
EWLVPP

SEQ ID NO: 122 - protein, 6 aa, Artificial Sequence
  Cadherin antagonist containing Trp- cell adhesion recognition sequence
EWLVAP

SEQ ID NO: 123 - protein, 6 aa, Artificial Sequence
  Cadherin antagonist containing Trp- cell adhesion recognition sequence
EWLMPP

SEQ ID NO: 124 - protein, 6 aa, Artificial Sequence
  Cadherin antagonist containing Trp- cell adhesion recognition sequence
EWLMAP

SEQ ID NO: 125 - protein, 4 aa, Artificial Sequence
  Cadherin antagonist containing Trp- cell adhesion recognition sequence
WLIP

SEQ ID NO: 126 - protein, 4 aa, Artificial Sequence
  Cadherin antagonist containing Trp- cell adhesion recognition sequence
WLIA

SEQ ID NO: 127 - protein, 4 aa, Artificial Sequence
  Cadherin antagonist containing Trp- cell adhesion recognition sequence
WLVP

SEQ ID NO: 128 - protein, 4 aa, Artificial Sequence
  Cadherin antagonist containing Trp- cell adhesion recognition sequence
WLVA

SEQ ID NO: 129 - protein, 4 aa, Artificial Sequence
  Cadherin antagonist containing Trp- cell adhesion recognition sequence
WLMP

SEQ ID NO: 130 - protein, 4 aa, Artificial Sequence
  Cadherin antagonist containing Trp- cell adhesion recognition sequence
WLMA

SEQ ID NO: 131 - protein, 5 aa, Artificial Sequence
  Cadherin antagonist containing Trp- cell adhesion recognition sequence
WLIPP

SEQ ID NO: 132 - protein, 5 aa, Artificial Sequence
  Cadherin antagonist containing Trp- cell adhesion recognition sequence
WLIAP

SEQ ID NO: 133 - protein, 5 aa, Artificial Sequence
  Cadherin antagonist containing Trp- cell adhesion recognition sequence
WLVPP

SEQ ID NO: 134 - protein, 5 aa, Artificial Sequence
  Cadherin antagonist containing Trp- cell adhesion recognition sequence
WLVAP

SEQ ID NO: 135 - protein, 5 aa, Artificial Sequence
  Cadherin antagonist containing Trp- cell adhesion recognition sequence
WLMPP

SEQ ID NO: 136 - protein, 5 aa, Artificial Sequence
  Cadherin antagonist containing Trp- cell adhesion recognition sequence
WLMAP

SEQ ID NO: 137 - protein, 4 aa, Artificial Sequence
  Cadherin antagonist containing Trp- cell adhesion recognition sequence
DWVL

SEQ ID NO: 138 - protein, 4 aa, Artificial Sequence
  Cadherin antagonist containing Trp- cell adhesion recognition sequence
DWIL

SEQ ID NO: 139 - protein, 4 aa, Artificial Sequence
  Cadherin antagonist containing Trp- cell adhesion recognition sequence
DWLL

SEQ ID NO: 140 - protein, 4 aa, Artificial Sequence
  Cadherin antagonist containing Trp- cell adhesion recognition sequence
EWVL

SEQ ID NO: 141 - protein, 4 aa, Artificial Sequence
  Cadherin antagonist containing Trp- cell adhesion recognition sequence
EWIL

SEQ ID NO: 142 - protein, 4 aa, Artificial Sequence
  Cadherin antagonist containing Trp- cell adhesion recognition sequence
EWLL

SEQ ID NO: 143 - protein, 5 aa, Artificial Sequence
  Cadherin antagonist containing Trp- cell adhesion recognition sequence
DWVLP

SEQ ID NO: 144 - protein, 5 aa, Artificial Sequence
  Cadherin antagonist containing Trp- cell adhesion recognition sequence
DWILP

SEQ ID NO: 145 - protein, 5 aa, Artificial Sequence
  Cadherin antagonist containing Trp- cell adhesion recognition sequence
DWLLP

SEQ ID NO: 146 - protein, 5 aa, Artificial Sequence
  Cadherin antagonist containing Trp- cell adhesion recognition sequence
EWVLP

SEQ ID NO: 147 - protein, 5 aa, Artificial Sequence
  Cadherin antagonist containing Trp- cell adhesion recognition sequence
EWILP

SEQ ID NO: 148 - protein, 5 aa, Artificial Sequence
  Cadherin antagonist containing Trp- cell adhesion recognition sequence
EWLLP

SEQ ID NO: 149 - protein, 5 aa, Artificial Sequence
  Cadherin antagonist containing Trp- cell adhesion recognition sequence
DWVLA

SEQ ID NO: 150 - protein, 5 aa, Artificial Sequence
  Cadherin antagonist containing Trp- cell adhesion recognition sequence
DWILA

SEQ ID NO: 151 - protein, 5 aa, Artificial Sequence
  Cadherin antagonist containing Trp- cell adhesion recognition sequence
DWLLA

SEQ ID NO: 152 - protein, 5 aa, Artificial Sequence
  Cadherin antagonist containing Trp- cell adhesion recognition sequence
EWVLA

SEQ ID NO: 153 - protein, 5 aa, Artificial Sequence
  Cadherin antagonist containing Trp- cell adhesion recognition sequence
EWILA

SEQ ID NO: 154 - protein, 5 aa, Artificial Sequence
  Cadherin antagonist containing Trp- cell adhesion recognition sequence
EWLLA

SEQ ID NO: 155 - protein, 6 aa, Artificial Sequence
  Cadherin antagonist containing Trp- cell adhesion recognition sequence
DWVLPP

SEQ ID NO: 156 - protein, 6 aa, Artificial Sequence
  Cadherin antagonist containing Trp- cell adhesion recognition sequence
DWILPP

SEQ ID NO: 157 - protein, 6 aa, Artificial Sequence
  Cadherin antagonist containing Trp- cell adhesion recognition sequence
DWLLPP

SEQ ID NO: 158 - protein, 6 aa, Artificial Sequence
  Cadherin antagonist containing Trp- cell adhesion recognition sequence
EWVLPP

SEQ ID NO: 159 - protein, 6 aa, Artificial Sequence
  Cadherin antagonist containing Trp- cell adhesion recognition sequence
EWILPP

SEQ ID NO: 160 - protein, 6 aa, Artificial Sequence
  Cadherin antagonist containing Trp- cell adhesion recognition sequence
EWLLPP

SEQ ID NO: 161 - protein, 6 aa, Artificial Sequence
  Cadherin antagonist containing Trp- cell adhesion recognition sequence
DWVLAP

SEQ ID NO: 162 - protein, 6 aa, Artificial Sequence
  Cadherin antagonist containing Trp- cell adhesion recognition sequence
DWILAP

SEQ ID NO: 163 - protein, 6 aa, Artificial Sequence
  Cadherin antagonist containing Trp- cell adhesion recognition sequence
DWLLAP

SEQ ID NO: 164 - protein, 6 aa, Artificial Sequence
  Cadherin antagonist containing Trp- cell adhesion recognition sequence
EWVLAP

SEQ ID NO: 165 - protein, 6 aa, Artificial Sequence
  Cadherin antagonist containing Trp- cell adhesion recognition sequence
EWILAP

SEQ ID NO: 166 - protein, 6 aa, Artificial Sequence
  Cadherin antagonist containing Trp- cell adhesion recognition sequence
EWLLAP

SEQ ID NO: 167 - protein, 4 aa, Artificial Sequence
  Cadherin antagonist containing Trp- cell adhesion recognition sequence
WVLP

SEQ ID NO: 168 - protein, 4 aa, Artificial Sequence
  Cadherin antagonist containing Trp- cell adhesion recognition sequence
WILP

SEQ ID NO: 169 - protein, 4 aa, Artificial Sequence
  Cadherin antagonist containing Trp- cell adhesion recognition sequence
WLLP

SEQ ID NO: 170 - protein, 4 aa, Artificial Sequence
  Cadherin antagonist containing Trp- cell adhesion recognition sequence
WVLA

SEQ ID NO: 171 - protein, 4 aa, Artificial Sequence
  Cadherin antagonist containing Trp- cell adhesion recognition sequence
WILA

SEQ ID NO: 172 - protein, 4 aa, Artificial Sequence
  Cadherin antagonist containing Trp- cell adhesion recognition sequence
WLLA

SEQ ID NO: 173 - protein, 5 aa, Artificial Sequence
  Cadherin antagonist containing Trp- cell adhesion recognition sequence
WVLPP

SEQ ID NO: 174 - protein, 5 aa, Artificial Sequence
  Cadherin antagonist containing Trp- cell adhesion recognition sequence
WILPP

SEQ ID NO: 175 - protein, 5 aa, Artificial Sequence
  Cadherin antagonist containing Trp- cell adhesion recognition sequence
WLLPP

SEQ ID NO: 176 - protein, 5 aa, Artificial Sequence
  Cadherin antagonist containing Trp- cell adhesion recognition sequence
WVLAP

SEQ ID NO: 177 - protein, 5 aa, Artificial Sequence
  Cadherin antagonist containing Trp- cell adhesion recognition sequence
WILAP

SEQ ID NO: 178 - protein, 5 aa, Artificial Sequence
  Cadherin antagonist containing Trp- cell adhesion recognition sequence
WLLAP

SEQ ID NO: 179 - protein, 6 aa, Artificial Sequence
  Cadherin antagonist containing Trp- cell adhesion recognition sequence
DWVIPP

SEQ ID NO: 180 - protein, 5 aa, Artificial Sequence
  Cadherin antagonist containing Trp- cell adhesion recognition sequence
DWVVA

SEQ ID NO: 181 - protein, 5 aa, Artificial Sequence
  Cadherin antagonist containing Trp- cell adhesion recognition sequence
EWVMP

SEQ ID NO: 182 - protein, 11 aa, Artificial Sequence
  Cadherin antagonist containing HAV binding motifcell adhesion recognition sequence
XFXIXXXXGXX

SEQ ID NO: 183 - protein, 11 aa, Artificial Sequence
  Cadherin antagonist containing HAV binding motifcell adhesion recognition sequence
WLXIXXXXGQI

SEQ ID NO: 184 - protein, 11 aa, Artificial Sequence
  Cadherin antagonist containing HAV binding motif
IFIINPISGQL

SEQ ID NO: 185 - protein, 11 aa, Artificial Sequence
  Cadherin antagonist containing HAV binding motif
IFILNPISGQL

SEQ ID NO: 186 - protein, 11 aa, Artificial Sequence
  Cadherin antagonist containing HAV binding motif
VFAVEKETGWL

SEQ ID NO: 187 - protein, 11 aa, Artificial Sequence
  Cadherin antagonist containing HAV binding motif
VFSINSMSGRM

SEQ ID NO: 188 - protein, 11 aa, Artificial Sequence
  Cadherin antagonist containing HAV binding motif
VFIIERETGWL

SEQ ID NO: 189 - protein, 11 aa, Artificial Sequence
  Cadherin antagonist containing HAV binding motif
VFTIEKESGWL

SEQ ID NO: 190 - protein, 11 aa, Artificial Sequence
  Cadherin antagonist containing HAV binding motif
VFNIDSMSGRM

SEQ ID NO: 191 - protein, 11 aa, Artificial Sequence
  Cadherin antagonist containing HAV binding motif
WLKIDSVNGQI

SEQ ID NO: 192 - protein, 11 aa, Artificial Sequence
  Cadherin antagonist containing HAV binding motif
WLKIDPVNGQI

SEQ ID NO: 193 - protein, 11 aa, Artificial Sequence
  Cadherin antagonist containing HAV binding motif
WLAMDPDSGQV

SEQ ID NO: 194 - protein, 11 aa, Artificial Sequence
  Cadherin antagonist containing HAV binding motif
WLHINATNGQI

SEQ ID NO: 195 - protein, 11 aa, Artificial Sequence
  Cadherin antagonist containing HAV binding motif
WLEINPDTGAI

SEQ ID NO: 196 - protein, 11 aa, Artificial Sequence
  Cadherin antagonist containing HAV binding motif
WLAVDPDSGQI

SEQ ID NO: 197 - protein, 11 aa, Artificial Sequence
  Cadherin antagonist containing HAV binding motif
WLEINPETGAI

SEQ ID NO: 198 - protein, 11 aa, Artificial Sequence
  Cadherin antagonist containing HAV binding motif
WLHINTSNGQI

SEQ ID NO: 199 - protein, 11 aa, Artificial Sequence
  Cadherin antagonist containing HAV binding motif
NLKIDPVNGQI

SEQ ID NO: 200 - protein, 10 aa, Artificial Sequence
  Cadherin antagonist containing HAV binding motif
LKIDPVNGQI

SEQ ID NO: 201 - protein, 7 aa, Artificial Sequence
  Cadherin antagonist containing HAV binding motif
INPISGQ

SEQ ID NO: 202 - protein, 7 aa, Artificial Sequence
  Cadherin antagonist containing HAV binding motif
LNPISGQ

SEQ ID NO: 203 - protein, 7 aa, Artificial Sequence
  Cadherin antagonist containing HAV binding motif
IDPVSGQ

SEQ ID NO: 204 - protein, 8 aa, Artificial Sequence
  Cadherin antagonist containing HAV binding motif
KIDPVNGQ

SEQ ID NO: 205 - protein, 5 aa, Artificial Sequence
  Cadherin antagonist containing HAV binding motif
PISGQ

SEQ ID NO: 206 - protein, 5 aa, Artificial Sequence
  Cadherin antagonist containing HAV binding motif
PVNGQ

SEQ ID NO: 207 - protein, 5 aa, Artificial Sequence
  Cadherin antagonist containing HAV binding motif
PVSGR

SEQ ID NO: 208 - protein, 5 aa, Artificial Sequence
  Cadherin antagonist containing HAV binding motif
IDPVN

SEQ ID NO: 209 - protein, 5 aa, Artificial Sequence
  Cadherin antagonist containing HAV binding motif
INPIS

SEQ ID NO: 210 - protein, 5 aa, Artificial Sequence
  Cadherin antagonist containing HAV binding motif
KIDPV

SEQ ID NO: 211 - protein, 9 aa, Artificial Sequence
  Non-classical cadherin cell adhesion recognition sequence
XFXXXXXXG

SEQ ID NO: 212 - protein, 4 aa, Artificial Sequence
  OB-cadherin antagonist
IDDK

SEQ ID NO: 213 - protein, 4 aa, Artificial Sequence
  OB-cadherin antagonist
DDKS

SEQ ID NO: 214 - protein, 5 aa, Artificial Sequence
  OB-cadherin antagonist
VIDDK

SEQ ID NO: 215 - protein, 5 aa, Artificial Sequence
  OB-cadherin antagonist
IDDKS

SEQ ID NO: 216 - protein, 6 aa, Artificial Sequence
  OB-cadherin antagonist
VIDDKS

SEQ ID NO: 217 - protein, 5 aa, Artificial Sequence
  OB-cadherin antagonist
DDKSG

SEQ ID NO: 218 - protein, 6 aa, Artificial Sequence
  OB-cadherin antagonist
IDDKSG

SEQ ID NO: 219 - protein, 7 aa, Artificial Sequence
  OB-cadherin antagonist
VIDDKSG

SEQ ID NO: 220 - protein, 6 aa, Artificial Sequence
  OB-cadherin antagonist
FVIDDK

SEQ ID NO: 221 - protein, 7 aa, Artificial Sequence
  OB-cadherin antagonist
FVIDDKS

SEQ ID NO: 222 - protein, 8 aa, Artificial Sequence
  OB-cadherin antagonist
FVIDDKSG

SEQ ID NO: 223 - protein, 7 aa, Artificial Sequence
  OB-cadherin antagonist
IFVIDDK

SEQ ID NO: 224 - protein, 8 aa, Artificial Sequence
  OB-cadherin antagonist
IFVIDDKS

SEQ ID NO: 225 - protein, 9 aa, Artificial Sequence
  OB-cadherin antagonist
IFVIDDKSG

SEQ ID NO: 226 - protein, 4 aa, Artificial Sequence
  OB-cadherin antagonist
IEEY

SEQ ID NO: 227 - protein, 4 aa, Artificial Sequence
  OB-cadherin antagonist
EEYT

SEQ ID NO: 228 - protein, 5 aa, Artificial Sequence
  OB-cadherin antagonist
VIEEY

SEQ ID NO: 229 - protein, 5 aa, Artificial Sequence
  OB-cadherin antagonist
IEEYT

SEQ ID NO: 230 - protein, 6 aa, Artificial Sequence
  OB-cadherin antagonist
VIEEYT

SEQ ID NO: 231 - protein, 5 aa, Artificial Sequence
  OB-cadherin antagonist
EEYTG

SEQ ID NO: 232 - protein, 6 aa, Artificial Sequence
  OB-cadherin antagonist
IEEYTG

SEQ ID NO: 233 - protein, 7 aa, Artificial Sequence
  OB-cadherin antagonist
VIEEYTG

SEQ ID NO: 234 - protein, 6 aa, Artificial Sequence
  OB-cadherin antagonist
FVIEEY

SEQ ID NO: 235 - protein, 7 aa, Artificial Sequence
  OB-cadherin antagonist
FVIEEYT

SEQ ID NO: 236 - protein, 8 aa, Artificial Sequence
  OB-cadherin antagonist
FVIEEYTG

SEQ ID NO: 237 - protein, 7 aa, Artificial Sequence
  OB-cadherin antagonist
FFVIEEY

SEQ ID NO: 238 - protein, 8 aa, Artificial Sequence
  OB-cadherin antagonist
FFVIEEYT

SEQ ID NO: 239 - protein, 9 aa, Artificial Sequence
  OB-cadherin antagonist
FFVIEEYTG

SEQ ID NO: 240 - protein, 4 aa, Artificial Sequence
  OB-cadherin antagonist
VEAQ

SEQ ID NO: 241 - protein, 4 aa, Artificial Sequence
  OB-cadherin antagonist
EAQT

SEQ ID NO: 242 - protein, 5 aa, Artificial Sequence
  OB-cadherin antagonist
SVEAQ

SEQ ID NO: 243 - protein, 5 aa, Artificial Sequence
  OB-cadherin antagonist
VEAQT

SEQ ID NO: 244 - protein, 6 aa, Artificial Sequence
  OB-cadherin antagonist
SVEAQT

SEQ ID NO: 245 - protein, 5 aa, Artificial Sequence
  OB-cadherin antagonist
EAQTG

SEQ ID NO: 246 - protein, 6 aa, Artificial Sequence
  OB-cadherin antagonist
VEAQTG

SEQ ID NO: 247 - protein, 7 aa, Artificial Sequence
  OB-cadherin antagonist
SVEAQTG

SEQ ID NO: 248 - protein, 6 aa, Artificial Sequence
  OB-cadherin antagonist
FSVEAQ

SEQ ID NO: 249 - protein, 7 aa, Artificial Sequence
  OB-cadherin antagonist
FSVEAQT

SEQ ID NO: 250 - protein, 8 aa, Artificial Sequence
  OB-cadherin antagonist
FSVEAQTG

SEQ ID NO: 251 - protein, 7 aa, Artificial Sequence
  OB-cadherin antagonist
YFSVEAQ

SEQ ID NO: 252 - protein, 8 aa, Artificial Sequence
  OB-cadherin antagonist
YFSVEAQT

SEQ ID NO: 253 - protein, 9 aa, Artificial Sequence
  OB-cadherin antagonist
YFSVEAQTG

SEQ ID NO: 254 - protein, 4 aa, Artificial Sequence
  VE-cadherin antagonist
VDAE

SEQ ID NO: 255 - protein, 4 aa, Artificial Sequence
  VE-cadherin antagonist
DAET

SEQ ID NO: 256 - protein, 5 aa, Artificial Sequence
  VE-cadherin antagonist
RVDAE

SEQ ID NO: 257 - protein, 5 aa, Artificial Sequence
  VE-cadherin antagonist
VDAET

SEQ ID NO: 258 - protein, 6 aa, Artificial Sequence
  VE-cadherin antagonist
RVDAET

SEQ ID NO: 259 - protein, 5 aa, Artificial Sequence
  VE-cadherin antagonist
DAETG

SEQ ID NO: 260 - protein, 6 aa, Artificial Sequence
  VE-cadherin antagonist
VDAETG

SEQ ID NO: 261 - protein, 7 aa, Artificial Sequence
  VE-cadherin antagonist
RVDAETG

SEQ ID NO: 262 - protein, 6 aa, Artificial Sequence
  VE-cadherin antagonist
FRVDAE

SEQ ID NO: 263 - protein, 7 aa, Artificial Sequence
  VE-cadherin antagonist
FRVDAET

SEQ ID NO: 264 - protein, 8 aa, Artificial Sequence
  VE-cadherin antagonist
FRVDAETG

SEQ ID NO: 265 - protein, 7 aa, Artificial Sequence
  VE-cadherin antagonist
VFRVDAE

SEQ ID NO: 266 - protein, 8 aa, Artificial Sequence
  VE-cadherin antagonist
VFRVDAET

SEQ ID NO: 267 - protein, 9 aa, Artificial Sequence
  VE-cadherin antagonist
VFRVDAETG

SEQ ID NO: 268 - protein, 6 aa, Artificial Sequence
  Atypical cadherin Trp-containing cell adhesion recognition sequence
XWXWNQ

SEQ ID NO: 269 - protein, 4 aa, Artificial Sequence
  Atypical cadherin Trp-containing cell adhesion recognition sequence
GWVW

SEQ ID NO: 270 - protein, 5 aa, Artificial Sequence
  Atypical cadherin Trp-containing cell adhesion recognition sequence
GWVWN

SEQ ID NO: 271 - protein, 6 aa, Artificial Sequence
  Atypical cadherin Trp-containing cell adhesion recognition sequence
GWVWNQ

SEQ ID NO: 272 - protein, 4 aa, Artificial Sequence
  Atypical cadherin Trp-containing cell adhesion recognition sequence
WVWN

SEQ ID NO: 273 - protein, 5 aa, Artificial Sequence
  Atypical cadherin Trp-containing cell adhesion recognition sequence
WVWNQ

SEQ ID NO: 274 - protein, 4 aa, Artificial Sequence
  Atypical cadherin Trp-containing cell adhesion recognition sequence
DWIW

SEQ ID NO: 275 - protein, 5 aa, Artificial Sequence
  Atypical cadherin Trp-containing cell adhesion recognition sequence
DWIWN

SEQ ID NO: 276 - protein, 6 aa, Artificial Sequence
  Atypical cadherin Trp-containing cell adhesion recognition sequence
DWIWNQ

SEQ ID NO: 277 - protein, 4 aa, Artificial Sequence
  Atypical cadherin Trp-containing cell adhesion recognition sequence
WIWN

SEQ ID NO: 278 - protein, 5 aa, Artificial Sequence
  Atypical cadherin Trp-containing cell adhesion recognition sequence
WIWNQ

SEQ ID NO: 279 - protein, 4 aa, Artificial Sequence
  Atypical cadherin Trp-containing cell adhesion recognition sequence
SWMW

SEQ ID NO: 280 - protein, 5 aa, Artificial Sequence
  Atypical cadherin Trp-containing cell adhesion recognition sequence
SWMWN

SEQ ID NO: 281 - protein, 6 aa, Artificial Sequence
  Atypical cadherin Trp-containing cell adhesion recognition sequence
SWMWNQ

SEQ ID NO: 282 - protein, 4 aa, Artificial Sequence
  Atypical cadherin Trp-containing cell adhesion recognition sequence
WMWN

SEQ ID NO: 283 - protein, 5 aa, Artificial Sequence
  Atypical cadherin Trp-containing cell adhesion recognition sequence
WMWNQ

SEQ ID NO: 284 - protein, 4 aa, Artificial Sequence
  Atypical cadherin Trp-containing cell adhesion recognition sequence
SWVW

SEQ ID NO: 285 - protein, 5 aa, Artificial Sequence
  Atypical cadherin Trp-containing cell adhesion recognition sequence
SWVWN

SEQ ID NO: 286 - protein, 6 aa, Artificial Sequence
  Atypical cadherin Trp-containing cell adhesion recognition sequence
SWVWNQ

SEQ ID NO: 287 - protein, 4 aa, Artificial Sequence
  Atypical cadherin Trp-containing cell adhesion recognition sequence
GWMW

SEQ ID NO: 288 - protein, 5 aa, Artificial Sequence
  Atypical cadherin Trp-containing cell adhesion recognition sequence
GWMWN

SEQ ID NO: 289 - protein, 6 aa, Artificial Sequence
  Atypical cadherin Trp-containing cell adhesion recognition sequence
GWMWNQ

SEQ ID NO: 290 - protein, 4 aa, Artificial Sequence
  Atypical cadherin Trp-containing cell adhesion recognition sequence
AWVI

SEQ ID NO: 291 - protein, 5 aa, Artificial Sequence
  Atypical cadherin Trp-containing cell adhesion recognition sequence
AWVIP

SEQ ID NO: 292 - protein, 6 aa, Artificial Sequence
  Atypical cadherin Trp-containing cell adhesion recognition sequence
AWVIPP

SEQ ID NO: 293 - protein, 4 aa, Artificial Sequence
  Atypical cadherin Trp-containing cell adhesion recognition sequence
WVIP

SEQ ID NO: 294 - protein, 5 aa, Artificial Sequence
  Atypical cadherin Trp-containing cell adhesion recognition sequence
WVIPP

SEQ ID NO: 295 - protein, 7 aa, Artificial Sequence
  Atypical cadherin Trp-containing cell adhesion recognition sequence
GWVWNQF

SEQ ID NO: 296 - protein, 8 aa, Artificial Sequence
  Atypical cadherin Trp-containing cell adhesion recognition sequence
GWVWNQFF

SEQ ID NO: 297 - protein, 9 aa, Artificial Sequence
  Atypical cadherin Trp-containing cell adhesion recognition sequence
GWVWNQFFV

SEQ ID NO: 298 - protein, 6 aa, Artificial Sequence
  Atypical cadherin Trp-containing cell adhesion recognition sequence
WVWNQF

SEQ ID NO: 299 - protein, 7 aa, Artificial Sequence
  Atypical cadherin Trp-containing cell adhesion recognition sequence
WVWNQFF

SEQ ID NO: 300 - protein, 8 aa, Artificial Sequence
  Atypical cadherin Trp-containing cell adhesion recognition sequence
WVWNQFFV

SEQ ID NO: 301 - protein, 4 aa, Artificial Sequence
  Atypical cadherin Trp-containing cell adhesion recognition sequence
RGWV

SEQ ID NO: 302 - protein, 5 aa, Artificial Sequence
  Atypical cadherin Trp-containing cell adhesion recognition sequence
RGWVW

SEQ ID NO: 303 - protein, 6 aa, Artificial Sequence
  Atypical cadherin Trp-containing cell adhesion recognition sequence
RGWVWN

SEQ ID NO: 304 - protein, 7 aa, Artificial Sequence
  Atypical cadherin Trp-containing cell adhesion recognition sequence
RGWVWNQ

SEQ ID NO: 305 - protein, 8 aa, Artificial Sequence
  Atypical cadherin Trp-containing cell adhesion recognition sequence
RGWVWNQF

SEQ ID NO: 306 - protein, 9 aa, Artificial Sequence
  Atypical cadherin Trp-containing cell adhesion recognition sequence
RGWVWNQFF

SEQ ID NO: 307 - protein, 10 aa, Artificial Sequence
  Atypical cadherin Trp-containing cell adhesion recognition sequence
RGWVWNQFFV

SEQ ID NO: 308 - protein, 4 aa, Artificial Sequence
  Atypical cadherin Trp-containing cell adhesion recognition sequence
KRGW

SEQ ID NO: 309 - protein, 5 aa, Artificial Sequence
  Atypical cadherin Trp-containing cell adhesion recognition sequence
KRGWV

SEQ ID NO: 310 - protein, 6 aa, Artificial Sequence
  Atypical cadherin Trp-containing cell adhesion recognition sequence
KRGWVW

SEQ ID NO: 311 - protein, 7 aa, Artificial Sequence
  Atypical cadherin Trp-containing cell adhesion recognition sequence
KRGWVWN

SEQ ID NO: 312 - protein, 8 aa, Artificial Sequence
  Atypical cadherin Trp-containing cell adhesion recognition sequence
KRGWVWNQ

SEQ ID NO: 313 - protein, 9 aa, Artificial Sequence
  Atypical cadherin Trp-containing cell adhesion recognition sequence
KRGWVWNQF

SEQ ID NO: 314 - protein, 10 aa, Artificial Sequence
  Atypical cadherin Trp-containing cell adhesion recognition sequence
KRGWVWNQFF

SEQ ID NO: 315 - protein, 11 aa, Artificial Sequence
  Atypical cadherin Trp-containing cell adhesion recognition sequence
KRGWVWNQFFV

SEQ ID NO: 316 - protein, 7 aa, Artificial Sequence
  Atypical cadherin Trp-containing cell adhesion recognition sequence
DWIWNQM

SEQ ID NO: 317 - protein, 8 aa, Artificial Sequence
  Atypical cadherin Trp-containing cell adhesion recognition sequence
DWIWNQMH

SEQ ID NO: 318 - protein, 9 aa, Artificial Sequence
  Atypical cadherin Trp-containing cell adhesion recognition sequence
DWIWNQMHI

SEQ ID NO: 319 - protein, 6 aa, Artificial Sequence
  Atypical cadherin Trp-containing cell adhesion recognition sequence
WIWNQM

SEQ ID NO: 320 - protein, 7 aa, Artificial Sequence
  Atypical cadherin Trp-containing cell adhesion recognition sequence
WIWNQMH

SEQ ID NO: 321 - protein, 8 aa, Artificial Sequence
  Atypical cadherin Trp-containing cell adhesion recognition sequence
WIWNQMHI

SEQ ID NO: 322 - protein, 4 aa, Artificial Sequence
  Atypical cadherin Trp-containing cell adhesion recognition sequence
RDWI

SEQ ID NO: 323 - protein, 5 aa, Artificial Sequence
  Atypical cadherin Trp-containing cell adhesion recognition sequence
RDWIW

SEQ ID NO: 324 - protein, 6 aa, Artificial Sequence
  Atypical cadherin Trp-containing cell adhesion recognition sequence
RDWIWN

SEQ ID NO: 325 - protein, 7 aa, Artificial Sequence
  Atypical cadherin Trp-containing cell adhesion recognition sequence
RDWIWNQ

SEQ ID NO: 326 - protein, 8 aa, Artificial Sequence
  Atypical cadherin Trp-containing cell adhesion recognition sequence
RDWIWNQM

SEQ ID NO: 327 - protein, 9 aa, Artificial Sequence
  Atypical cadherin Trp-containing cell adhesion recognition sequence
RDWIWNQMH

SEQ ID NO: 328 - protein, 10 aa, Artificial Sequence
  Atypical cadherin Trp-containing cell adhesion recognition sequence
RDWIWNQMHI

SEQ ID NO: 329 - protein, 4 aa, Artificial Sequence
  Atypical cadherin Trp-containing cell adhesion recognition sequence
KRDW

SEQ ID NO: 330 - protein, 5 aa, Artificial Sequence
  Atypical cadherin Trp-containing cell adhesion recognition sequence
KRDWI

SEQ ID NO: 331 - protein, 6 aa, Artificial Sequence
  Atypical cadherin Trp-containing cell adhesion recognition sequence
KRDWIW

SEQ ID NO: 332 - protein, 7 aa, Artificial Sequence
  Atypical cadherin Trp-containing cell adhesion recognition sequence
KRDWIWN

SEQ ID NO: 333 - protein, 8 aa, Artificial Sequence
  Atypical cadherin Trp-containing cell adhesion recognition sequence
KRDWIWNQ

SEQ ID NO: 334 - protein, 9 aa, Artificial Sequence
  Atypical cadherin Trp-containing cell adhesion recognition sequence
KRDWIWNQM

SEQ ID NO: 335 - protein, 10 aa, Artificial Sequence
  Atypical cadherin Trp-containing cell adhesion recognition sequence
KRDWIWNQMH

SEQ ID NO: 336 - protein, 11 aa, Artificial Sequence
  Atypical cadherin Trp-containing cell adhesion recognition sequence
KRDWIWNQMHI

SEQ ID NO: 337 - protein, 7 aa, Artificial Sequence
  Atypical cadherin Trp-containing cell adhesion recognition sequence
SWMWNQF

SEQ ID NO: 338 - protein, 8 aa, Artificial Sequence
  Atypical cadherin Trp-containing cell adhesion recognition sequence
SWMWNQFF

SEQ ID NO: 339 - protein, 9 aa, Artificial Sequence
  Atypical cadherin Trp-containing cell adhesion recognition sequence
SWMWNQFFL

SEQ ID NO: 340 - protein, 6 aa, Artificial Sequence
  Atypical cadherin Trp-containing cell adhesion recognition sequence
WMWNQF

SEQ ID NO: 341 - protein, 7 aa, Artificial Sequence
  Atypical cadherin Trp-containing cell adhesion recognition sequence
WMWNQFF

SEQ ID NO: 342 - protein, 8 aa, Artificial Sequence
  Atypical cadherin Trp-containing cell adhesion recognition sequence
WMWNQFFL

SEQ ID NO: 343 - protein, 4 aa, Artificial Sequence
  Atypical cadherin Trp-containing cell adhesion recognition sequence
RSWM

SEQ ID NO: 344 - protein, 5 aa, Artificial Sequence
  Atypical cadherin Trp-containing cell adhesion recognition sequence
RSWMW

SEQ ID NO: 345 - protein, 6 aa, Artificial Sequence
  Atypical cadherin Trp-containing cell adhesion recognition sequence
RSWMWN

SEQ ID NO: 346 - protein, 7 aa, Artificial Sequence
  Atypical cadherin Trp-containing cell adhesion recognition sequence
RSWMWNQ

SEQ ID NO: 347 - protein, 8 aa, Artificial Sequence
  Atypical cadherin Trp-containing cell adhesion recognition sequence
RSWMWNQF

SEQ ID NO: 348 - protein, 9 aa, Artificial Sequence
  Atypical cadherin Trp-containing cell adhesion recognition sequence
RSWMWNQFF

SEQ ID NO: 349 - protein, 10 aa, Artificial Sequence
  Atypical cadherin Trp-containing cell adhesion recognition sequence
RSWMWNQFFL

SEQ ID NO: 350 - protein, 4 aa, Artificial Sequence
  Atypical cadherin Trp-containing cell adhesion recognition sequence
KRSW

SEQ ID NO: 351 - protein, 5 aa, Artificial Sequence
  Atypical cadherin Trp-containing cell adhesion recognition sequence
KRSWM

SEQ ID NO: 352 - protein, 6 aa, Artificial Sequence
  Atypical cadherin Trp-containing cell adhesion recognition sequence
KRSWMW

SEQ ID NO: 353 - protein, 7 aa, Artificial Sequence
  Atypical cadherin Trp-containing cell adhesion recognition sequence
KRSWMWN

SEQ ID NO: 354 - protein, 8 aa, Artificial Sequence
  Atypical cadherin Trp-containing cell adhesion recognition sequence
KRSWMWNQ

SEQ ID NO: 355 - protein, 9 aa, Artificial Sequence
  Atypical cadherin Trp-containing cell adhesion recognition sequence
KRSWMWNQF

SEQ ID NO: 356 - protein, 10 aa, Artificial Sequence
  Atypical cadherin Trp-containing cell adhesion recognition sequence
KRSWMWNQFF

SEQ ID NO: 357 - protein, 11 aa, Artificial Sequence
  Atypical cadherin Trp-containing cell adhesion recognition sequence
KRSWMWNQFFL

SEQ ID NO: 358 - protein, 7 aa, Artificial Sequence
  Atypical cadherin Trp-containing cell adhesion recognition sequence
SWVWNQF

SEQ ID NO: 359 - protein, 8 aa, Artificial Sequence
  Atypical cadherin Trp-containing cell adhesion recognition sequence
SWVWNQFF

SEQ ID NO: 360 - protein, 9 aa, Artificial Sequence
  Atypical cadherin Trp-containing cell adhesion recognition sequence
SWVWNQFFV

SEQ ID NO: 361 - protein, 6 aa, Artificial Sequence
  Atypical cadherin Trp-containing cell adhesion recognition sequence
WVWNQF

SEQ ID NO: 362 - protein, 7 aa, Artificial Sequence
  Atypical cadherin Trp-containing cell adhesion recognition sequence
WVWNQFF

SEQ ID NO: 363 - protein, 8 aa, Artificial Sequence
  Atypical cadherin Trp-containing cell adhesion recognition sequence
WVWNQFFV

SEQ ID NO: 364 - protein, 4 aa, Artificial Sequence
  Atypical cadherin Trp-containing cell adhesion recognition sequence
RSWV

SEQ ID NO: 365 - protein, 5 aa, Artificial Sequence
  Atypical cadherin Trp-containing cell adhesion recognition sequence
RSWVW

SEQ ID NO: 366 - protein, 6 aa, Artificial Sequence
  Atypical cadherin Trp-containing cell adhesion recognition sequence
RSWVWN

SEQ ID NO: 367 - protein, 7 aa, Artificial Sequence
  Atypical cadherin Trp-containing cell adhesion recognition sequence
RSWVWNQ

SEQ ID NO: 368 - protein, 8 aa, Artificial Sequence
  Atypical cadherin Trp-containing cell adhesion recognition sequence
RSWVWNQF

SEQ ID NO: 369 - protein, 9 aa, Artificial Sequence
  Atypical cadherin Trp-containing cell adhesion recognition sequence
RSWVWNQFF

SEQ ID NO: 370 - protein, 10 aa, Artificial Sequence
  Atypical cadherin Trp-containing cell adhesion recognition sequence
RSWVWNQFFV

SEQ ID NO: 371 - protein, 5 aa, Artificial Sequence
  Atypical cadherin Trp-containing cell adhesion recognition sequence
KRSWV

SEQ ID NO: 372 - protein, 6 aa, Artificial Sequence
  Atypical cadherin Trp-containing cell adhesion recognition sequence
KRSWVW

SEQ ID NO: 373 - protein, 7 aa, Artificial Sequence
  Atypical cadherin Trp-containing cell adhesion recognition sequence
KRSWVWN

SEQ ID NO: 374 - protein, 8 aa, Artificial Sequence
  Atypical cadherin Trp-containing cell adhesion recognition sequence
KRSWVWNQ

SEQ ID NO: 375 - protein, 9 aa, Artificial Sequence
  Atypical cadherin Trp-containing cell adhesion recognition sequence
KRSWVWNQF

SEQ ID NO: 376 - protein, 10 aa, Artificial Sequence
  Atypical cadherin Trp-containing cell adhesion recognition sequence
KRSWVWNQFF

SEQ ID NO: 377 - protein, 11 aa, Artificial Sequence
  Atypical cadherin Trp-containing cell adhesion recognition sequence
KRSWVWNQFFV

SEQ ID NO: 378 - protein, 7 aa, Artificial Sequence
  Atypical cadherin Trp-containing cell adhesion recognition sequence
GWVWNQM

SEQ ID NO: 379 - protein, 8 aa, Artificial Sequence
  Atypical cadherin Trp-containing cell adhesion recognition sequence
GWVWNQMF

SEQ ID NO: 380 - protein, 9 aa, Artificial Sequence
  Atypical cadherin Trp-containing cell adhesion recognition sequence
GWVWNQMFV

SEQ ID NO: 381 - protein, 8 aa, Artificial Sequence
  Atypical cadherin Trp-containing cell adhesion recognition sequence
RGWVWNQM

SEQ ID NO: 382 - protein, 9 aa, Artificial Sequence
  Atypical cadherin Trp-containing cell adhesion recognition sequence
RGWVWNQMF

SEQ ID NO: 383 - protein, 10 aa, Artificial Sequence
  Atypical cadherin Trp-containing cell adhesion recognition sequence
RGWVWNQMFV

SEQ ID NO: 384 - protein, 9 aa, Artificial Sequence
  Atypical cadherin Trp-containing cell adhesion recognition sequence
KRGWVWNQM

SEQ ID NO: 385 - protein, 11 aa, Artificial Sequence
  Atypical cadherin Trp-containing cell adhesion recognition sequence
KRGWVWNQMFV

SEQ ID NO: 386 - protein, 9 aa, Artificial Sequence
  Atypical cadherin Trp-containing cell adhesion recognition sequence
GWVWNQFFL

SEQ ID NO: 387 - protein, 10 aa, Artificial Sequence
  Atypical cadherin Trp-containing cell adhesion recognition sequence
RGWVWNQFFL

SEQ ID NO: 388 - protein, 11 aa, Artificial Sequence
  Atypical cadherin Trp-containing cell adhesion recognition sequence
KRGWVWNQFFL

SEQ ID NO: 389 - protein, 7 aa, Artificial Sequence
  Atypical cadherin Trp-containing cell adhesion recognition sequence
AWVIPPI

SEQ ID NO: 390 - protein, 8 aa, Artificial Sequence
  Atypical cadherin Trp-containing cell adhesion recognition sequence
AWVIPPIS

SEQ ID NO: 391 - protein, 9 aa, Artificial Sequence
  Atypical cadherin Trp-containing cell adhesion recognition sequence
AWVIPPISV

SEQ ID NO: 392 - protein, 6 aa, Artificial Sequence
  Atypical cadherin Trp-containing cell adhesion recognition sequence
WVIPPI

SEQ ID NO: 393 - protein, 7 aa, Artificial Sequence
  Atypical cadherin Trp-containing cell adhesion recognition sequence
WVIPPIS

SEQ ID NO: 394 - protein, 8 aa, Artificial Sequence
  Atypical cadherin Trp-containing cell adhesion recognition sequence
WVIPPISV

SEQ ID NO: 395 - protein, 4 aa, Artificial Sequence
  Atypical cadherin Trp-containing cell adhesion recognition sequence
RAWV

SEQ ID NO: 396 - protein, 5 aa, Artificial Sequence
  Atypical cadherin Trp-containing cell adhesion recognition sequence
RAWVI

SEQ ID NO: 397 - protein, 6 aa, Artificial Sequence
  Atypical cadherin Trp-containing cell adhesion recognition sequence
RAWVIP

SEQ ID NO: 398 - protein, 7 aa, Artificial Sequence
  Atypical cadherin Trp-containing cell adhesion recognition sequence
RAWVIPP

SEQ ID NO: 399 - protein, 8 aa, Artificial Sequence
  Atypical cadherin Trp-containing cell adhesion recognition sequence
RAWVIPPI

SEQ ID NO: 400 - protein, 9 aa, Artificial Sequence
  Atypical cadherin Trp-containing cell adhesion recognition sequence
RAWVIPPIS

SEQ ID NO: 401 - protein, 10 aa, Artificial Sequence
  Atypical cadherin Trp-containing cell adhesion recognition sequence
RAWVIPPISV

SEQ ID NO: 402 - protein, 4 aa, Artificial Sequence
  Atypical cadherin Trp-containing cell adhesion recognition sequence
KRAW

SEQ ID NO: 403 - protein, 5 aa, Artificial Sequence
  Atypical cadherin Trp-containing cell adhesion recognition sequence
KRAWV

SEQ ID NO: 404 - protein, 6 aa, Artificial Sequence
  Atypical cadherin Trp-containing cell adhesion recognition sequence
KRAWVI

SEQ ID NO: 405 - protein, 7 aa, Artificial Sequence
  Atypical cadherin Trp-containing cell adhesion recognition sequence
KRAWVIP

SEQ ID NO: 406 - protein, 8 aa, Artificial Sequence
  Atypical cadherin Trp-containing cell adhesion recognition sequence
KRAWVIPP

SEQ ID NO: 407 - protein, 9 aa, Artificial Sequence
  Atypical cadherin Trp-containing cell adhesion recognition sequence
KRAWVIPPI

SEQ ID NO: 408 - protein, 10 aa, Artificial Sequence
  Atypical cadherin Trp-containing cell adhesion recognition sequence
KRAWVIPPIS

SEQ ID NO: 409 - protein, 4 aa, Artificial Sequence
  Atypical cadherin Trp-containing cell adhesion recognition sequence
VWNQ

SEQ ID NO: 410 - protein, 5 aa, Artificial Sequence
  Atypical cadherin Trp-containing cell adhesion recognition sequence
VWNQM

SEQ ID NO: 411 - protein, 5 aa, Artificial Sequence
  Atypical cadherin Trp-containing cell adhesion recognition sequence
VWNQF

SEQ ID NO: 412 - protein, 6 aa, Artificial Sequence
  Atypical cadherin Trp-containing cell adhesion recognition sequence
VWNQMF

SEQ ID NO: 413 - protein, 6 aa, Artificial Sequence
  Atypical cadherin Trp-containing cell adhesion recognition sequence
VWNQFF

SEQ ID NO: 414 - protein, 4 aa, Artificial Sequence
  Atypical cadherin Trp-containing cell adhesion recognition sequence
WNQM

SEQ ID NO: 415 - protein, 4 aa, Artificial Sequence
  Atypical cadherin Trp-containing cell adhesion recognition sequence
WNQF

SEQ ID NO: 416 - protein, 5 aa, Artificial Sequence
  Atypical cadherin Trp-containing cell adhesion recognition sequence
WNQFF

SEQ ID NO: 417 - protein, 4 aa, Artificial Sequence
  Atypical cadherin Trp-containing cell adhesion recognition sequence
IWNQ

SEQ ID NO: 418 - protein, 5 aa, Artificial Sequence
  Atypical cadherin Trp-containing cell adhesion recognition sequence
IWNQM

SEQ ID NO: 419 - protein, 6 aa, Artificial Sequence
  Atypical cadherin Trp-containing cell adhesion recognition sequence
IWNQMH

SEQ ID NO: 420 - protein, 4 aa, Artificial Sequence
  Atypical cadherin Trp-containing cell adhesion recognition sequence
WNQM

SEQ ID NO: 421 - protein, 5 aa, Artificial Sequence
  Atypical cadherin Trp-containing cell adhesion recognition sequence
WNQMH

SEQ ID NO: 422 - protein, 4 aa, Artificial Sequence
  Atypical cadherin Trp-containing cell adhesion recognition sequence
MWNQ

SEQ ID NO: 423 - protein, 5 aa, Artificial Sequence
  Atypical cadherin Trp-containing cell adhesion recognition sequence
MWNQF

SEQ ID NO: 424 - protein, 6 aa, Artificial Sequence
  Atypical cadherin Trp-containing cell adhesion recognition sequence
MWNQFF

SEQ ID NO: 425 - protein, 5 aa, Artificial Sequence
  Calcium bindin motif
DXNDN

SEQ ID NO: 426 - protein, 4 aa, Artificial Sequence
  Calcium binding motif
LDRE

SEQ ID NO: 427 - protein, 15 aa, Artificial Sequence
  N-cadherin CAR sequence which modulating agents may be directed against.
FHLRAHAVDINGNQV

SEQ ID NO: 428 - protein, 48 aa, Artificial Sequence
  occludin CAR sequence region
GVNPTAQSSGSLYGSQIYALCNQFYTPAATGLYVDQYLYHYCVVDPQE

SEQ ID NO: 429 - protein, 4 aa, Artificial Sequence
  Modulating agent
LYHY

(19) United States
(12) Patent Application Publication
(10) Pub. No.: US 2009/0291967 A1
(43) Pub. Date: Nov. 26, 2009

(54) SMALL MOLECULE MODULATORS OF CELL ADHESION

(75) Inventors: Mukur Gupta, Morrisville, NC (US); Brian Huber, Durham, NC (US); Orest W. Blaschuk, Westmount (CA)

Correspondence Address: SEED INTELLECTUAL PROPERTY LAW GROUP PLLC, 701 FIFTH AVE, SUITE 5400, SEATTLE, WA, 98104, US

(73) Assignee: ADHEREX TECHNOLOGIES, INC., Ottawa (CA)

(21) Appl. No.: 12/398,649

(22) Filed: Mar. 5, 2009

Related U.S. Application Data
(60) Provisional application No. 61/034,075, filed on Mar. 5, 2008.

(51) Int. Cl. A61K 31/4965; C07D 249/08; C07D 271/06; C07D 401/04; A61K 31/4196; A61K 31/4245; A61P 35/00

(52) U.S. Cl. 514/255.05; 548/262.2; 548/132; 544/405; 514/384; 514/364

(57) ABSTRACT

Compounds, particularly compounds having activity as modulators of cadherin-mediated cell adhesion having the following structure:

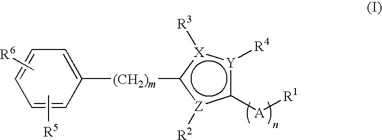

or a pharmaceutically acceptable salt, stereoisomer or prodrug thereof,
wherein R¹, R², R³, R⁴, R⁵, R⁶, A, X, Y, Z, m and n are as defined herein. Methods associated with preparation and use of the same, as well as pharmaceutical compositions containing the same, are also disclosed.

### CROSS-REFERENCE TO RELATED APPLICATION

This application claims the benefit under 35 U.S.C. § 119(e) of U.S. Provisional Patent Application No. 61/034,075, filed Mar. 5, 2008; where this provisional application is incorporated herein by reference in its entirety.

### BACKGROUND

1. Technical Field

The present invention generally relates to compounds, particularly compounds active as modulators of cadherin-mediated cell adhesion, as well as to methods associated with the same.

2. Description of the Related Art

Cell adhesion is a complex process that is important for maintaining tissue integrity and generating physical and permeability barriers within the body. All tissues are divided into discrete compartments, each of which is composed of a specific cell type that adheres to similar cell types. Such adhesion triggers the formation of intercellular junctions (i.e., readily definable contact sites on the surfaces of adjacent cells that are adhering to one another), also known as tight junctions, gap junctions and belt desmosomes. The formation of such junctions gives rise to physical and permeability barriers that restrict the free passage of cells and other biological substances from one tissue compartment to another. For example, the blood vessels of all tissues are composed of endothelial cells. In order for components in the blood to enter a given tissue compartment, they must first pass from the lumen of a blood vessel through the barrier formed by the endothelial cells of that vessel. Similarly, in order for substances to enter the body via the gut, the substances must first pass through a barrier formed by the epithelial cells of that tissue. To enter the blood via the skin, both epithelial and endothelial cell layers must be crossed.

Cell adhesion is mediated by specific cell surface adhesion molecules (CAMs). There are many different families of CAMs, including the immunoglobulin, integrin, selectin and cadherin superfamilies, and each cell type expresses a unique combination of these molecules. Cadherins are a rapidly expanding family of calcium-dependent CAMs (Munro et al., In: Cell Adhesion and Invasion in Cancer Metastasis, P. Brodt, ed., pp. 17-34, RG Landes Co. (Austin Tex., 1996). The classical cadherins (abbreviated CADs) are integral membrane glycoproteins that generally promote cell adhesion through homophilic interactions (a CAD on the surface of one cell binds to an identical CAD on the surface of another cell), although CADs also appear to be capable of forming heterotypic complexes with one another under certain circumstances and with lower affinity. Cadherins have been shown to regulate epithelial, endothelial, neural and cancer cell adhesion, with different CADs expressed on different cell types. N (neural)-cadherin is predominantly expressed by neural cells, endothelial cells and a variety of cancer cell types. E (epithelial)-cadherin is predominantly expressed by epithelial cells. Other CADs are P (placental)-cadherin, which is found in human skin and R (retinal)-cadherin. A detailed discussion of the classical cadherins is provided in Munro S B et al., 1996, In: Cell Adhesion and Invasion in Cancer Metastasis, P. Brodt, ed., pp. 17-34 (RG Landes Company, Austin Tex.).

The structures of the CADs are generally similar. As illustrated in FIG. 1, CADs are composed of five extracellular domains (EC1-EC5), a single hydrophobic domain (TM) that transverses the plasma membrane (PM), and two cytoplasmic domains (CP1 and CP2). The calcium binding motifs DXNDN, DXD and LDRE are interspersed throughout the extracellular domains. The first extracellular domain (EC1) contains the classical cadherin cell adhesion recognition (CAR) sequence, HAV (His-Ala-Val), along with flanking sequences on either side of the CAR sequence that may play a role in conferring specificity. Synthetic peptides containing the CAR sequence and antibodies directed against the CAR sequence have been shown to inhibit CAD-dependent processes (Munro et al., supra; Blaschuk et al., J. Mol. Biol. 211:679-82, 1990; Blaschuk et al., Develop. Biol. 139:227-29, 1990; Alexander et al., J. Cell Physiol. 156:610-18, 1993). The three-dimensional solution and crystal structures of the EC1 domain have been determined (Overduin et al., Science 267:386-389, 1995; Shapiro et al., Nature 374:327-337, 1995).

Although cell adhesion is required for certain normal physiological functions, there are situations in which cell adhesion is undesirable. Many pathologies (such as autoimmune and inflammatory diseases) involve abnormal cellular adhesion. Cell adhesion may also play a role in graft rejection. In such circumstances, modulation of cell adhesion may be desirable. For example, N-cadherin is known to promote neurite outgrowth via a homophilic binding mechanism. N-cadherin is normally found on both the advancing growth cone and on cellular substrates, and the inhibition of N-cadherin function results in diminished neurite outgrowth. Such inhibition may be the result of pathology or injury involving severed neuronal connections and/or spinal cord damage. In such cases, enhancement of N-cadherin mediated neurite outgrowth would be beneficial. However, previous attempts to promote neurite outgrowth have achieved limited success due, in part, to difficulties associated with maintaining continuous growth over a particular defined region.

Although a number of peptide-based modulators of N-cadherin have been described (e.g., peptides comprising the CAR sequence, HAV), there remains a need in the art for alternative compounds that modulate cell adhesion without certain of the disadvantages that may be associated with some peptide-based therapeutics. The present invention fulfills this need and further provides other related advantages.

### BRIEF SUMMARY

In brief, this invention is generally directed to compounds having activity as cell adhesion modulators, as well as to methods for their preparation and use, and to pharmaceutical compositions containing the same. Such compounds have the following general structure (I):

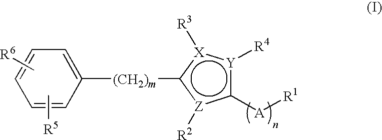

or a pharmaceutically acceptable salt, stereoisomer or prodrug thereof, wherein R¹, R², R³, R⁴, R⁵, R⁶, X, Y, Z, m and n are as defined below.

The compounds of the present invention have utility over a wide range of therapeutic applications, and may be used to treat a variety of conditions, including conditions benefiting from modulation of cell adhesion, in both men and women, as well as a mammal in general (also referred to herein as a “subject”).

In still a further embodiment, pharmaceutical compositions are disclosed containing one or more compounds of formula (I) in combination with a pharmaceutically acceptable carrier and/or diluent.

These and other aspects of the invention will be apparent upon reference to the following detailed description. To this end, various references are set forth herein which describe in more detail certain background information, procedures, compounds and/or compositions, and are each hereby incorporated by reference in their entirety.

### DETAILED DESCRIPTION

As mentioned above, compounds are disclosed having the following general structure (I):

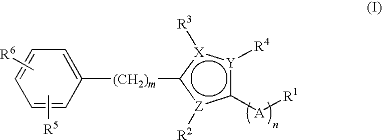

or a pharmaceutically acceptable salt, stereoisomer or prodrug thereof, wherein

A is —NH—, —O— or —S—;

X and Y are independently nitrogen, oxygen or carbon;

Z is nitrogen or oxygen;

R¹ is hydrogen, optionally substituted alkyl, optionally substituted aryl or optionally substituted heterocycle;

R², and R⁴ are independently either present or absent and when present are independently hydrogen, optionally substituted alkyl, optionally substituted aryl or optionally substituted heterocycle, except that R², R³ and R⁴ cannot be carboxyl;

R⁵ and R⁶ are independently hydrogen, halogen, optionally substituted alkyl, optionally substituted aryl, optionally substituted heterocycle or —OR⁷, or R⁵ and R⁶, when attached to adjacent carbons of the phenyl ring, join to form an optionally substituted, fused aryl group;

R⁷ is hydrogen, lower alkyl, aryl or alkylaryl;

m and n are independently 0 or 1; and

the ring formed by X, Y and Z is aromatic.

As used herein, the above terms have the following meaning:

“Alkyl” means a straight chain or branched, noncyclic or cyclic, unsaturated or saturated aliphatic hydrocarbon containing from 1 to 8 carbon atoms, while the term “lower alkyl” has the same meaning as alkyl but contains from 1 to 4 carbon atoms. Representative saturated straight chain alkyls include methyl, ethyl, n-propyl, n-butyl, n-pentyl, n-hexyl, and the like; while saturated branched alkyls include isopropyl, sec-butyl, isobutyl, tert-butyl, isopentyl, and the like. Representative saturated cyclic alkyls include cyclopropyl, cyclobutyl, cyclopentyl, cyclohexyl, and the like; while unsaturated cyclic alkyls include cyclopentenyl and cyclohexenyl, and the like. Unsaturated alkyls contain at least one double or triple bond between adjacent carbon atoms (referred to as an “alkenyl” or “alkynyl”, respectively). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1 butynyl, and the like.

“Aryl” means an aromatic carbocyclic moiety such as phenyl or naphthyl.

“Alkylaryl” means any alkyl group as defined herein which is further substituted with an aryl group. Alkylaryls include benzyl and the like.

“Heterocycle” means a 5- to 7-membered monocyclic, or 7- to 10-membered bicyclic, heterocyclic ring which is either saturated, unsaturated, or aromatic, and which contains from 1 to 4 heteroatoms independently selected from nitrogen, oxygen and sulfur, and wherein the nitrogen and sulfur heteroatoms may be optionally oxidized, and the nitrogen heteroatom may be optionally quaternized, including bicyclic rings in which any of the above heterocycles are fused to a benzene ring. The heterocycle may be attached via any heteroatom or carbon atom. Heterocycles include heteroaryls as defined below. Thus, in addition to the heteroaryls listed below, heterocycles also include morpholinyl, pyrrolidinonyl, pyrrolidinyl, piperidinyl, piperizynyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, tetrahydropyrimidinyl, tetrahydrothiophenyl, tetrahydrothiopyranyl, and the like.

“Heteroaryl” means an aromatic heterocycle ring of 5- to 10 members and having at least one heteroatom selected from nitrogen, oxygen and sulfur, and containing at least 1 carbon atom, including both mono- and bicyclic ring systems. Representative heteroaryls are pyridyl, furyl, benzofuranyl, thiophenyl, benzothiophenyl, quinolinyl, pyrrolyl, indolyl, oxazolyl, benzoxazolyl, imidazolyl, benzimidazolyl, thiazolyl, benzothiazolyl, isoxazolyl, pyrazolyl, isothiazolyl, pyridazinyl, pyrimidinyl, pyrazinyl, triazinyl, cinnolinyl, phthalazinyl, and quinazolinyl.

“Halogen” means fluoro, chloro, bromo and iodo.

The terms “optionally substituted alkyl,” “optionally substituted aryl” and “optionally substituted heterocycle” means that, when substituted, at least one hydrogen atom is replaced with a substituent. In the case of an oxo substituent (═O) two hydrogen atoms are replaced. In this regard, substituents include oxo, halogen, heterocycle, —CN, —OR^{x}, —NR^{x}R^{y}, —NR^{x}C(═O)R^{y}, —NR^{x}SO₂R^{y}, —C(═O)R^{x}, —C(═O)OR^{x}, —C(═O)NR^{x}R^{y}, —SOₙR^{x} and —SOₙNR^{x}R^{y}, wherein n is 0, 1 or 2, R^{x} and R^{y} are the same or different and independently hydrogen, alkyl or heterocycle, and each of said alkyl and heterocycle substituents may be further substituted with one or more of oxo, halogen, hydroxy, cyano, alkyl, alkoxy, heterocycle, —NR^{x}R^{y}, —NR^{x}C(═O)R^{y}, —NR^{x}SO₂R^{y}, —C(═O)R^{x}, —C(═O)OR^{x}, —C(═O)NR^{x}R^{y}, —SOₙR^{x} and —SOₙNR^{x}R^{y}.

In some embodiments, substituents of an optionally substituted aryl group may join to form a fused ring. In these embodiments any two of the substituents, when attached to adjacent atoms of the aryl group, may be taken together with the atoms to which they are attached to form a fused aryl ring, wherein the fused aryl ring may be substituted with one or more substituents as defined above.

In one embodiment of structure (I), A is —S—, X, Y and Z are nitrogen, m is 0 and n is 1, and compounds of this invention have the following structure (II):

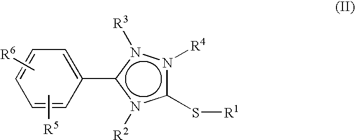

In further embodiments of structure (II), R¹ is hydrogen or methyl, at least two of R², R³ and R⁴ are absent and at least one of R², R³ and R⁴ is hydrogen, methyl or ethyl.

In another embodiment of structure (I), A is —O—, X, Y and Z are nitrogen, m is 0 and n is 1, and compounds of this invention have the following structure (III):

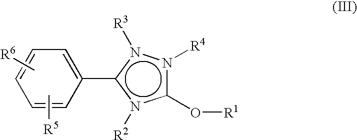

In further embodiments of structure (III), R¹ is hydrogen or methyl, at least two of R², R³ and R⁴ are absent and at least one of R², R³ and R⁴ is hydrogen.

In another embodiment of structure (I), A is —NH—, X, Y and Z are nitrogen, m is 0 and n is 1, and compounds of this invention have the following structure (IV):

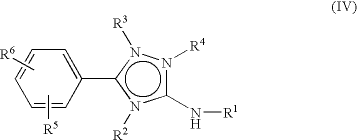

In further embodiments of structure (IV), R¹ is hydrogen, at least two of R², R³ and R⁴ are absent and at least one of R², R³ and R⁴ is hydrogen.

In another embodiment of structure (I), A is —S—, X and Y are nitrogen, Z is oxygen, m is 0 and n is 1, and compounds of this invention have the following structure (V):

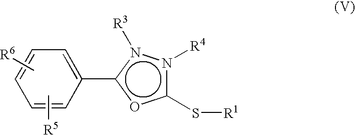

In further embodiments of structure (V), R¹ is hydrogen and R³ and R⁴ are absent.

In another embodiment of structure (I), A is —NH—, X and Y are nitrogen, Z is oxygen, m is 0 and n is 1, and compounds of this invention have the following structure (VI):

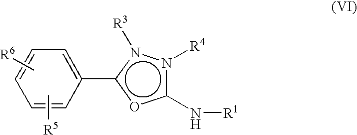

In further embodiments of structure (VI), R¹ is hydrogen and R³ and R⁴ are absent.

In another embodiment of structure (I), X, Y and Z are nitrogen and m and n are 0, and compounds of this invention have the following structure (VII):

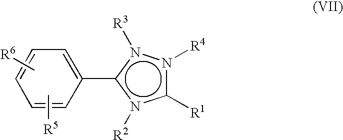

In further embodiments of structure (VII), R¹ is hydrogen or optionally substituted alkyl, at least two of R², R³ and R⁴ are absent and at least one of R², R³ and R⁴ is hydrogen.

In further embodiments of structure (VII), R¹ is methyl, at least two of R², R³ and R⁴ are absent and at least one of R², R³ and R⁴ is hydrogen.

In other embodiments of structure (VII), R¹ is optionally substituted aryl, at least two of R², R³ and R⁴ are absent and at least one of R², R³ and R⁴ is hydrogen.

In other embodiments of structure (VII), R¹ is optionally substituted heterocycle, at least two of R², R³ and R⁴ are absent and at least one of R², R³ and R⁴ is hydrogen.

In another embodiment of structure (I), X and Y are nitrogen, Z is oxygen and m and n are 0, and compounds of this invention have the following structure (VIII):

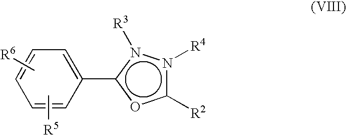

In further embodiments of structure (VIII), R¹ is methyl and R³ and R⁴ are absent.

In another embodiment of structure (I), m and n are 0 and either Y and Z are nitrogen, X is oxygen and R³ is absent or X and Z are nitrogen, Y is oxygen and R⁴ is absent as shown by structures (IX) and (X):

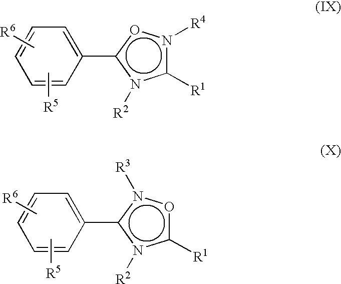

In further embodiments of structures (IX) and (X), R¹ is methyl and R², R³ and R⁴ are all absent.

In another embodiment of structure (I), m and n are 0 and either Y and Z are nitrogen and X is carbon or X and Z are nitrogen and Y is carbon as shown by structures (XI) and (XII):

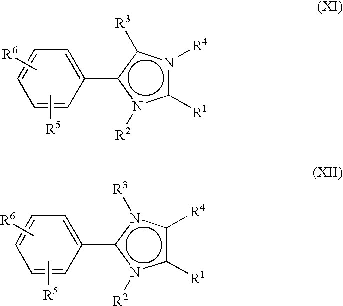

In further embodiments of structure (XI), R¹ is methyl, R² and R³ are hydrogen and R⁴ is absent.

In further embodiments of structure (XII), R¹ is methyl, R² and R⁴ are hydrogen and R³ is absent.

In another embodiment of structure (I), X, Y and Z are nitrogen, m is 1 and n is 0, and compounds of this invention have the following structure (XIII):

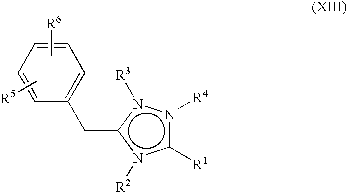

In further embodiments of structure (XIII), R¹ is methyl, R² is hydrogen and R³ and R⁴ are absent.

In further embodiments of structure (I), at least one of R⁵ and R⁶ has the following structure (where the wavy line indicates the point of attachment to the phenyl ring):

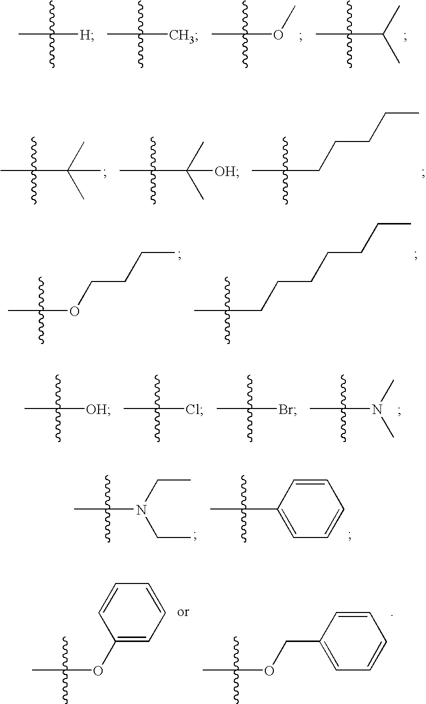

In further embodiments of structure (I), R⁵ and R⁶, when attached to adjacent atoms of the phenyl group, are taken together with the carbon atoms to which they are attached to form an optionally substituted, fused phenyl ring as shown by structures (XIV) and (XV) (where A represents an optionally substituted, fused phenyl ring):

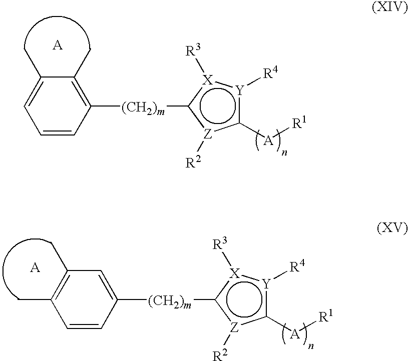

In further embodiments of structure (I), n is O and R¹ has the following structure (where the wavy line indicates the point of attachment to the ring):

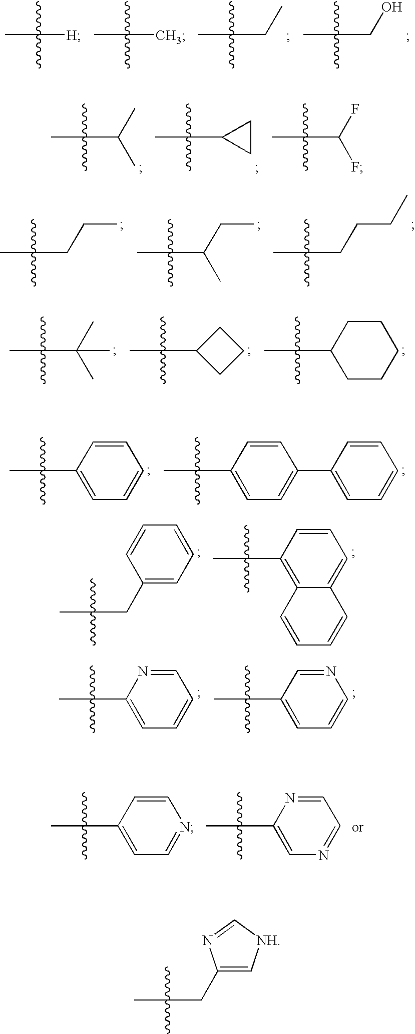

In further embodiments of structure (I), Z is nitrogen and R² has the following structure (where the wavy line indicates the point of attachment to the nitrogen atom):

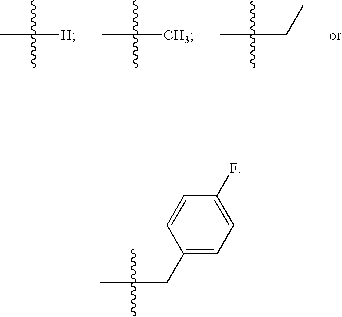

In further embodiments, pharmaceutical compositions comprising a compound of formula (I) and a pharmaceutically acceptable carrier or diluent are provided.

In other embodiments, a method is provided for modulating cadherin mediated cell adhesion in a subject comprising the step of administering to a subject in need of such treatment a therapeutically effective amount of a composition comprising a compound of formula (I).

In further embodiments, methods are provided for reducing unwanted cellular adhesion in a mammal, comprising administering to a mammal a cell adhesion modulating agent that inhibits cadherin-mediated cell adhesion, wherein the modulating agent comprises a compound of formula (I).

In other embodiments, the compound of formula (I) may, but need not, be linked to a targeting agent.

In other embodiments, methods are provided for enhancing the delivery of a drug to a tumor in a mammal, comprising administering to a mammal: (a) a cell adhesion modulating agent that inhibits cadherin-mediated cell adhesion, wherein the modulating agent comprises a compound of formula (I); and (b) a drug.

In more specific embodiments the tumors include, for example, bladder tumors, ovarian tumors and melanomas.

In other specific embodiments, the compound of formula (I) may be administered to the tumor or systemically.

In more embodiments, methods are provided for inhibiting the development of a cancer in a mammal, comprising administering to a mammal a cell adhesion modulating agent that inhibits cadherin-mediated cell adhesion, wherein the modulating agent comprises a compound of formula (I).

In more specific embodiments, the cancers include, for example, carcinomas, leukemias and melanomas.

In other embodiments, the invention provides methods for inhibiting angiogenesis in a mammal, comprising administering to a mammal a modulating agent that inhibits cadherin-mediated cell adhesion, wherein the modulating agent comprises a compound of formula (I).

In more specific embodiments, cancers include, for example, carcinomas, leukemias and melanomas.

In other embodiments, the invention provides methods for enhancing drug delivery to the central nervous system of a mammal, comprising administering to a mammal a modulating agent that inhibits cadherin-mediated cell adhesion, wherein the modulating agent comprises a compound of formula (I).

In other specific embodiments, the present invention provides methods for enhancing wound healing in a mammal, comprising contacting a wound in a mammal with a modulating agent that enhances cadherin-mediated cell adhesion, wherein the modulating agent comprises a compound of formula (I).

In other embodiments, the invention provides methods for enhancing adhesion of foreign tissue implanted within a mammal, comprising contacting a site of implantation of foreign tissue in a mammal with a modulating agent that enhances cadherin-mediated cell adhesion, wherein the modulating agent comprises a compound of formula (I).

In other embodiments, the present invention further provides methods for modulating the immune system of a mammal, comprising administering to a mammal a cell adhesion modulating agent that inhibits cadherin-mediated cell adhesion, wherein the modulating agent comprises a compound of formula (I).

In further embodiments, the invention provides methods for increasing vasopermeability in a mammal, comprising administering to a mammal a cell adhesion modulating agent that inhibits cadherin-mediated cell adhesion, wherein the modulating agent comprises a compound of formula (I).

In other embodiments, the present invention provides methods for treating a demyelinating neurological disease, such as multiple sclerosis, in a mammal, comprising administering to a mammal: (a) a cell adhesion modulating agent that inhibits cadherin-mediated cell adhesion, wherein the modulating agent comprises a compound of formula (I); and (b) one or more cells capable of replenishing an oligodendrocyte population.

In more specific embodiments, suitable cells include, for example, Schwann cells, oligodendrocyte progenitor cells and oligodendrocytes.

In other embodiments, the present invention further provides methods for inhibiting synaptic stability in a mammal, comprising administering to a mammal a cell adhesion modulating agent that inhibits cadherin-mediated cell adhesion, wherein the modulating agent comprises a compound of formula (I).

In further embodiments, the invention provides methods for modulating neurite outgrowth, comprising contacting a neuron with a modulating agent that comprises a compound of formula (I).

In more specific embodiments, neurite outgrowth may be inhibited or enhanced, and/or may be directed.

In other embodiments, the present invention provides methods for treating spinal cord injuries in a mammal, comprising administering to a mammal a cell adhesion modulating agent that enhances neurite outgrowth, wherein the modulating agent comprises a compound of formula (I).

In more specific embodiments, neurite outgrowth may be inhibited or enhanced, and/or directed.

In other embodiments, methods are provided for treating macular degeneration in a mammal, comprising administering to a mammal a cell adhesion modulating agent that enhances classical cadherin-mediated cell adhesion, wherein the modulating agent comprises a compound of formula (I).

In more embodiments, methods are provided for facilitating migration of an N-cadherin expressing cell on astrocytes, comprising contacting an N-cadherin expressing cell with: (a) a cell adhesion modulating agent that inhibits cadherin-mediated cell adhesion, wherein the modulating agent comprises a compound of formula (I); and (b) one or more astrocytes.

In more specific embodiments the N-cadherin expressing cells may be, for example, a Schwann cell, oligodendrocyte progenitor cell or oligodendrocyte.

In further embodiments, the invention provides kits for administering a drug via the skin of a mammal, comprising: (a) a skin patch; and (b) a cell adhesion modulating agent comprising a compound of formula (I).

In other embodiments methods for modulating classical cadherin-mediated intercellular adhesion, comprising contacting a classical cadherin-expressing cell with a composition comprising a compound of formula (I) are provided.

The compounds of the present invention may be prepared by known organic synthesis techniques, including the methods described in more detail in the Examples. In general, the compounds of structure (I) above may be made by the following Reaction Schemes 1-13, wherein all substituents are as defined above unless indicated otherwise.

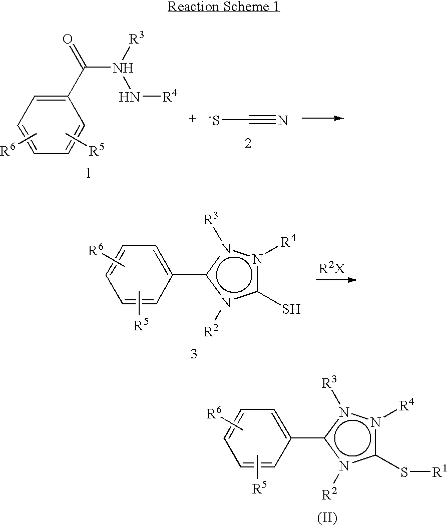

Compounds of structure (II) can be synthesized by methods known to those skilled in the art. For example, referring to Reaction Scheme 1, benzoylhydrazine 1 thiocyanate 2 can be purchased or prepared using methods known to those skilled in the art and reacted together to produce triazole 3. The sulfur group of triazole 3 can optionally be further functionalized using methods known to those skilled in the art, for example by reaction with R²X (X=halo), to obtain compounds of formula (II).

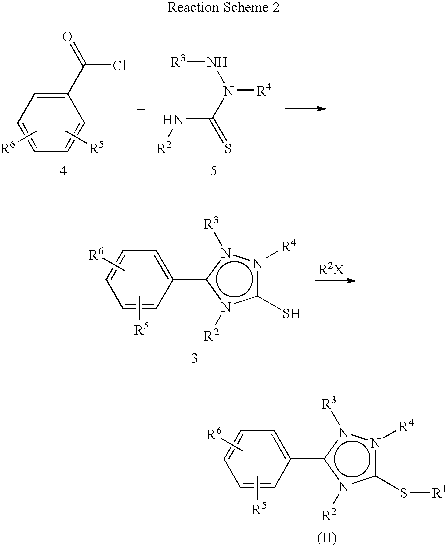

Alternatively, compounds of structure (II) can be synthesized by other methods. For example, referring to Reaction Scheme 2, benzoyl chloride 4 and thiosemicarbazide 5 can be purchased or synthesized using methods known to those skilled in the art and reacted together to produce triazole 3. The sulfur group of triazole 3 can optionally be further functionalized using methods known to those skilled in the art, for example by reaction with R²X (X=halo), to obtain compounds of formula (II).

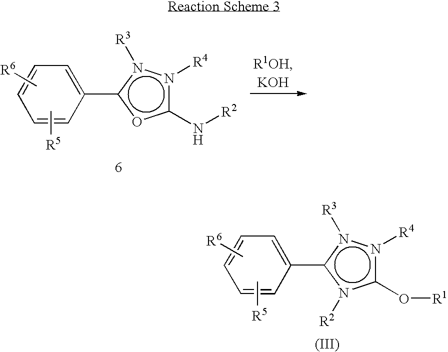

Compounds of structure (III) can be synthesized by methods known to those skilled in the art. For example, referring to Reaction Scheme 3, oxadiazole 6 can be purchased or prepared as described herein or by other methods known to those skilled in the art and reacted with an appropriate alcohol and a base, such as potassium hydroxide, to obtain compounds of formula (III).

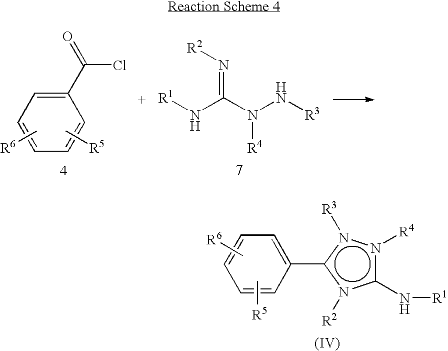

Compounds of structure (IV) can be synthesized by methods known to those skilled in the art. For example, referring to Reaction Scheme 4, benzoyl chloride 4 and aminoguanidine 7 can be purchased or prepared by methods known to those skilled in the art and reacted together in the presence of a base, such as sodium hydroxide, in a solvent, such as pyridine, to obtain compounds of formula (IV).

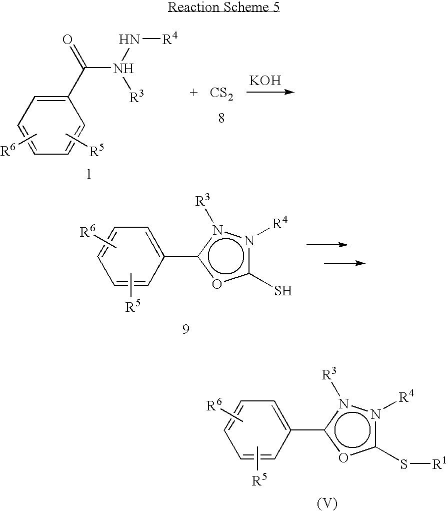

Compounds of structure (V) can be synthesized by methods known to those skilled in the art. For example, referring to Reaction Scheme 5, benzhydrazide 1 and can be purchased or prepared by methods known to those skilled in the art and reacted with carbon disulfide 8 in the presence of a base, such as potassium hydroxide, in a solvent, such as ethanol, to obtain compounds of formula 9. The sulfur group of 9 can optionally be further functionalized using methods known to those skilled in the art to obtain compounds of formula (V).

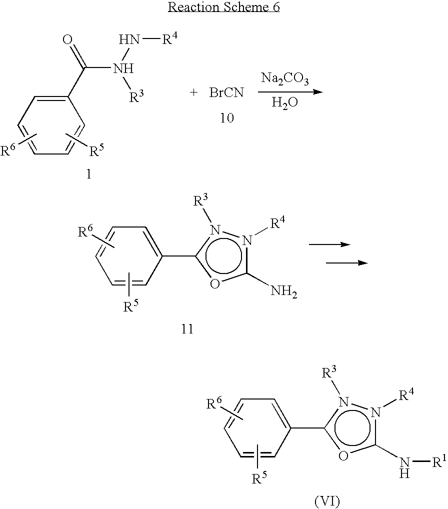

Compounds of structure (VI) can be synthesized by methods known to those skilled in the art. For example, referring to Reaction Scheme 6, benzhydrazide 1 and can be purchased or prepared by methods known to those skilled in the art and reacted with cyanogen bromide 10 in the presence of a base, such as sodium carbonate, in a solvent, such as water, to obtain compounds of formula II. The nitrogen group of 11 can optionally be further functionalized using methods known to those skilled in the art to obtain compounds of formula (VI).

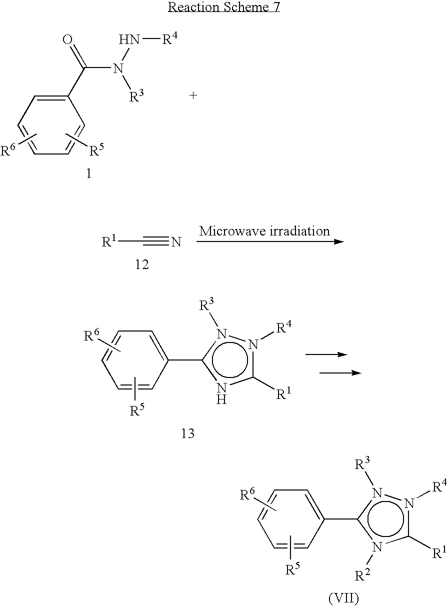

Compounds of structure (VII) can be synthesized by methods known to those skilled in the art. For example, referring to Reaction Scheme 7, benzhydrazide 1 and nitrile 12 can be purchased or prepared by methods known to those skilled in the art and subjected to microwave irradiation in the presence of a base, such as potassium carbonate, in a solvent, such as n-butyl alcohol, to obtain compounds of formula 13. The nitrogen group of 13 can optionally be further functionalized using methods known to those skilled in the art to obtain compounds of formula (VII).

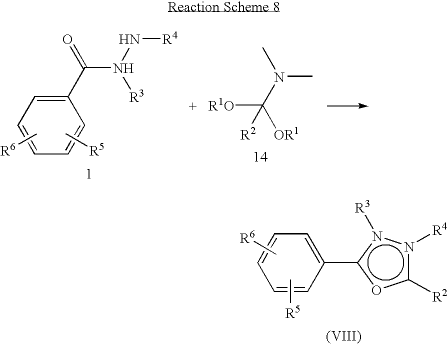

Compounds of structure (VIII) can be synthesized by methods known to those skilled in the art. For example, referring to Reaction Scheme 8, benzhydrazide 1 and alkylacetamidodialkylacetal 14 can be purchased or prepared by methods known to those skilled in the art in the presence of an acid, such as acetic acid, in a solvent, such as acetonitrile, to obtain compounds of formula (VIII).

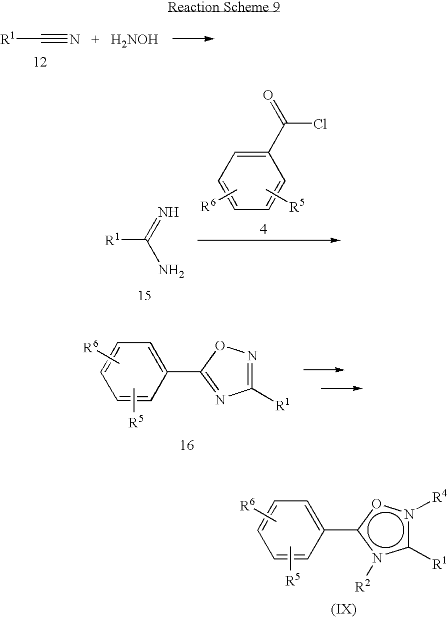

Compounds of structure (IX) can be synthesized by methods known to those skilled in the art. For example, referring to Reaction Scheme 9, nitrile 12 can be purchased or prepared by methods known to those skilled in the art and reacted with hydroxylamine to obtain amidine 15. Amidine 15 can be further reacted with benzoyl chloride 4 in a solvent, such as pyridine, to produce compounds of formula 16. The nitrogen groups of 16 can optionally be further functionalized using methods known to those skilled in the art to obtain compounds of formula (IX).

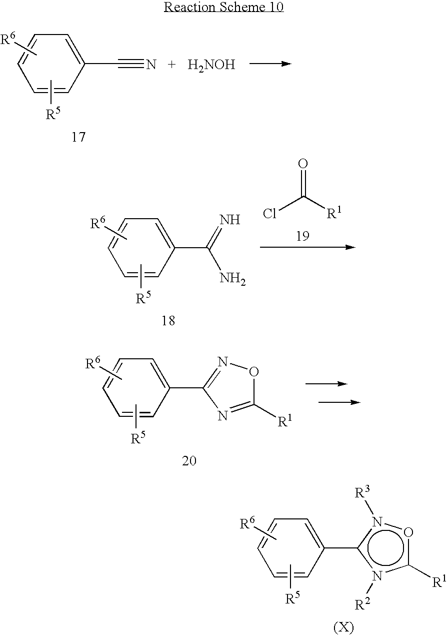

Compounds of structure (X) can be synthesized by methods known to those skilled in the art. For example, referring to Reaction Scheme 10, benzonitrile 17 can be purchased or prepared by methods known to those skilled in the art and reacted with a hydroxylamine to obtain benzamidine 18. Benzamidine 18 can be further reacted with acyl chloride 19 in a solvent, such as pyridine, to produce compounds of formula 20. The nitrogen groups of 20 can optionally be further functionalized using methods known to those skilled in the art to obtain compounds of formula (X).

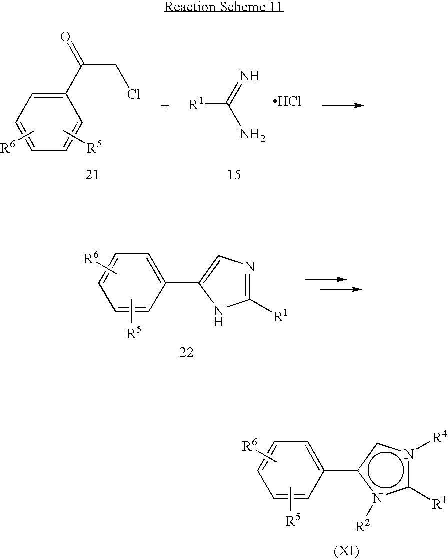

Compounds of structure (XI) can be synthesized by methods known to those skilled in the art. For example, referring to Reaction Scheme 11, chloride 21 and amidine 15 can be purchased or prepared by methods known to those skilled in the art and reacted together in a solvent, such as pyridine, in the presence of a base, such as potassium carbonate, to produce compounds of formula 22. The nitrogen groups of 22 can optionally be further functionalized using methods known to those skilled in the art to obtain compounds of formula (XI).

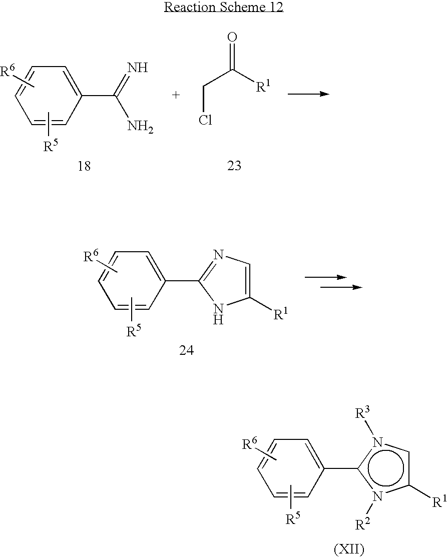

Compounds of structure (XII) can be synthesized by methods known to those skilled in the art. For example, referring to Reaction Scheme 12, benzamidine 18 and chloride 23 can be purchased or prepared by methods known to those skilled in the art and reacted together in a solvent, such as water, in the presence of a base, such as potassium bicarbonate, to produce compounds of formula 24. The nitrogen groups of 24 can optionally be further functionalized using methods known to those skilled in the art to obtain compounds of formula (XII).

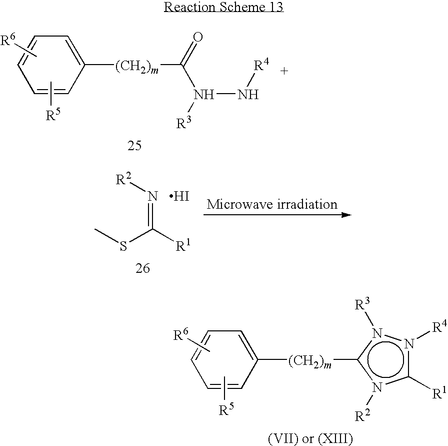

Compounds of structure (VII) or (XIII) can be synthesized by methods known to those skilled in the art. For example, referring to Reaction Scheme 13, benzhydrazide 25 and isothioamide 26 can be purchased or prepared by methods known to those skilled in the art and can be subjected to microwave irradiation in the presence of silica gel in a solvent, such as triethylamine, in the presence of an acid, such as ammonium acetate, to produce compounds of formula (VII) (m=0) or (XIII) (m=1).

### Evaluating Activity of Candidate Compounds

As noted above, compounds of formula (I) are capable of modulating (i.e., enhancing or inhibiting) classical cadherin-mediated cell adhesion. The ability of a modulating agent to modulate cell adhesion may generally be evaluated in vitro by assaying the effect on one or more of the following: (1) neurite outgrowth, (2) adhesion between endothelial cells, (3) adhesion between epithelial cells (e.g., normal rat kidney cells and/or human skin) and/or (4) adhesion between cancer cells. In general, a modulating agent is an inhibitor of cell adhesion if, within one or more of these representative assays, contact of the test cells with the modulating agent results in a discernible disruption of cell adhesion. Modulating agents that enhance cell adhesion are considered to be modulators of cell adhesion if they are capable of enhancing neurite outgrowth as described below and/or are capable of promoting cell adhesion, as judged by plating assays to assess epithelial cell adhesion to a modulating agent attached to a support material, such as tissue culture plastic. For modulating agents that affect N-cadherin mediated functions, assays involving endothelial or cancer cell adhesion or neurite outgrowth are preferred.

Within a representative neurite outgrowth assay, neurons may be cultured on a monolayer of cells (e.g., 3T3) that express N-cadherin. Neurons grown on such cells (under suitable conditions and for a sufficient period of time) extend longer neurites than neurons cultured on cells that do not express N-cadherin. For example, neurons may be cultured on monolayers of 3T3 cells transfected with cDNA encoding N-cadherin essentially as described by Doherty and Walsh, Curr. Op. Neurobiol. 4:49-55, 1994; Williams et al., Neuron 13:583-594, 1994; Hall et al., Cell Adhesion and Commun. 3:441-450, 1996; Doherty and Walsh, Mol. Cell. Neurosci. 8:99-111, 1994; and Safell et al., Neuron 18:231-242, 1997. Briefly, monolayers of control 3T3 fibroblasts and 3T3 fibroblasts that express N-cadherin may be established by overnight culture of 80,000 cells in individual wells of an 8-chamber well tissue culture slide. 3000 cerebellar neurons isolated from post-natal day 3 mouse brains may be cultured for 18 hours on the various monolayers in control media (SATO/2% FCS), or media supplemented with various concentrations of the modulating agent or control peptide. The cultures may then be fixed and stained for GAP43, which specifically binds to the neurons and their neurites. The length of the longest neurite on each GAP43 positive neuron may be measured by computer assisted morphometry. Additional neurite outgrowth assays for evaluating or confirming activity can include those described, for example, in Lagenaur et al., (Proc. Natl. Acad. Sci. USA 84: 7753-7757, 1987) and Hamburger et al. (J. Morphol. 88, 49-92, 1951).

A modulating agent that modulates N-cadherin-mediated cell adhesion may inhibit or enhance such neurite outgrowth. Under the conditions described above, the presence of 500 μg/mL of a modulating agent that disrupts neural cell adhesion should, in certain embodiments, result in a decrease in the mean neurite length by at least 50%, relative to the length in the absence of modulating agent or in the presence of a negative control peptide. Alternatively, the presence of 500 μg/mL of a modulating agent that enhances neural cell adhesion should, in certain embodiments, result in an increase in the mean neurite length by at least 50%.

Within one representative cell adhesion assay, the addition of a modulating agent to cells that express a cadherin results in disruption of cell adhesion. A “cadherin-expressing cell,” as used herein, may be any type of cell that expresses at least one cadherin on the cell surface at a detectable level, using standard techniques such as immunocytochemical protocols (Blaschuk and Farookhi, Dev. Biol. 136:564-567, 1989). Cadherin-expressing cells include endothelial (e.g., bovine pulmonary artery endothelial cells), epithelial and/or cancer cells (e.g., the human ovarian cancer cell line SKOV3 (ATCC #HTB-77)). For example, such cells may be plated under standard conditions that permit cell adhesion in the presence and absence of modulating agent (e.g., 500 μg/mL). Disruption of cell adhesion may be determined visually within 24 hours, by observing retraction of the cells from one another.

For use within one such assay, bovine pulmonary artery endothelial cells may be harvested by sterile ablation and digestion in 0.1% collagenase (type II; Worthington Enzymes, Freehold, N.J.). Cells may be maintained in Dulbecco's minimum essential medium supplemented with 10% fetal calf serum and 1% antibiotic-antimycotic at 37° C. in 7% CO₂ in air. Cultures may be passaged weekly in trypsin-EDTA and seeded onto tissue culture plastic at 20,000 cells/cm². Endothelial cultures may be used at 1 week in culture, which is approximately 3 days after culture confluency is established. The cells may be seeded onto coverslips and treated (e.g., for 30 minutes) with modulating agent or a control compound at, for example, 500 μg/ml and then fixed with 1% paraformaldehyde. As noted above, disruption of cell adhesion may be determined visually within 24 hours, by observing retraction of the cells from one another. This assay evaluates the effect of a modulating agent on N-cadherin mediated cell adhesion.

Within another such assay, the effect of a modulating agent on normal rat kidney (NRK) cells may be evaluated. According to a representative procedure, NRK cells (ATCC #1571-CRL) may be plated at 10-20,000 cells per 35 mm tissue culture flasks containing DMEM with 10% FCS and sub-cultured periodically (Laird et al., J. Cell Biol. 131:1193-1203, 1995). Cells may be harvested and replated in 35 mm tissue culture flasks containing 1 mm coverslips and incubated until 50-65% confluent (24-36 hours). At this time, coverslips may be transferred to a 24-well plate, washed once with fresh DMEM and exposed to modulating agent at a concentration of, for example, 1 mg/mL for 24 hours. Fresh modulating agent may then be added, and the cells left for an additional 24 hours. Cells may be fixed with 100% methanol for 10 minutes and then washed three times with PBS. Coverslips may be blocked for 1 hour in 2% BSA/PBS and incubated for a further 1 hour in the presence of mouse anti-E-cadherin antibody (Transduction Labs, 1:250 dilution). Primary and secondary antibodies may be diluted in 2% BSA/PBS. Following incubation in the primary antibody, coverslips may be washed three times for 5 minutes each in PBS and incubated for 1 hour with donkey anti-mouse antibody conjugated to fluorescein (diluted 1:200). Following further washes in PBS (3×5 min) coverslips can be mounted and viewed by confocal microscopy.

In the absence of modulating agent, NRK cells form characteristic tightly adherent monolayers with a cobblestone morphology in which cells display a polygonal shape. NRK cells that are treated with a modulating agent that disrupts E-cadherin mediated cell adhesion may assume a non-polygonal and elongated morphology (i.e., a fibroblast-like shape) within 48 hours of treatment with 1 mg/mL of modulating agent. Gaps appear in confluent cultures of such cells. In addition, 1 mg/mL of such a modulating agent reproducibly induces a readily apparent reduction in cell surface staining of E-cadherin, as judged by immunofluorescence microscopy (Laird et al., J. Cell Biol. 131:1193-1203, 1995), of at least 75% within 48 hours.

A third cell adhesion assay involves evaluating the effect of a modulating agent on permeability of adherent epithelial and/or endothelial cell layers. For example, the effect on permeability of human skin may be evaluated. Such skin may be derived from a natural source or may be synthetic. Human abdominal skin for use in such assays may generally be obtained from humans at autopsy within 24 hours of death. Briefly, a cyclic peptide and a test marker (e.g., the fluorescent markers Oregon Green™ and Rhodamine Green™ Dextran) may be dissolved in a sterile buffer, and the ability of the marker to penetrate through the skin and into a receptor fluid may be measured using a Franz Cell apparatus (Franz, Curr. Prob. Dermatol. 7:58-68, 1978; Franz, J. Invest. Dermatol. 64:190-195, 1975). In general, a modulating agent that enhances the permeability of human skin results in a statistically significant increase in the amount of marker in the receptor compartment after 6-48 hours in the presence of 500 μg/mL modulating agent. This assay evaluates the effect of a modulating agent on E-cadherin mediated cell adhesion.

Alternatively, cells that do not naturally express a cadherin may be used within such assays. Such cells may be stably transfected with a polynucleotide (e.g., a cDNA) encoding a classical cadherin of interest, such that the cadherin is expressed on the surface of the cell. Transfection of cells for use in cell adhesion assays may be performed using standard techniques and published cadherin sequences. Expression of the cadherin may be confirmed by assessing adhesion of the transfected cells, in conjunction with immunocytochemical techniques using antibodies directed against the cadherin of interest. The stably transfected cells that aggregate, as judged by light microscopy, following transfection express sufficient levels of the cadherin. Preferred cells for use in such assays include L cells, which do not detectably adhere in the absence of transfection (Nagafuchi et al., Nature 329:341-343, 1987). Following transfection of L cells with a cDNA encoding a cadherin, aggregation may be observed. Modulating agents that detectably inhibit such aggregation may be used to modulate functions mediated by the cadherin. Such assays have been used for numerous nonclassical cadherins, including OB-cadherin (Okazaki et al., J. Biol. Chem. 269:12092-98, 1994), cadherin-5 (Breier et al., Blood 87:630-641, 1996), cadherin-6 (Mbalaviele et al., J. Cell. Biol. 141:1467-1476, 1998), cadherin-8 (Kido et al., Genomics 48:186-194, 1998), cadherin-15 (Shimoyama et al., J. Biol. Chem. 273:10011-10018, 1998), PB-cadherin (Sugimoto et al., J. Biol. Chem. 271:11548-11556, 1996), LI-cadherin (Kreft et al., J. Cell. Biol. 136:1109-1121, 1997), protocadherin 42 and 43 (Sano et al., EMBO J. 12:2249-2256, 1993) and desmosomal cadherins (Marcozzi et al., J. Cell. Sci. 111:495-509, 1998). It will be apparent to those of ordinary skill in the art that assays may be performed in a similar manner for classical cadherins. In general, a modulating agent that is a compound of formula (I) and that modulates adhesion of a cell that expresses the same cadherin is considered to modulate a function mediated by the cadherin.

The compounds of the present invention may generally be utilized as the free base. Alternatively, the compounds of this invention may be used in the form of acid addition salts. Acid addition salts of the free amino compounds of the present invention may be prepared by methods well known in the art, and may be formed from organic and inorganic acids. Suitable organic acids include maleic, fumaric, benzoic, ascorbic, succinic, methanesulfonic, acetic, oxalic, propionic, tartaric, salicylic, citric, gluconic, lactic, mandelic, cinnamic, aspartic, stearic, palmitic, glycolic, glutamic, and benzenesulfonic acids. Suitable inorganic acids include hydrochloric, hydrobromic, sulfuric, phosphoric, and nitric acids. Thus, the term “pharmaceutically acceptable salt” of structure (I) is intended to encompass any and all acceptable salt forms.

In addition, prodrugs are also included within the context of this invention. Prodrugs are any covalently bonded carriers that release a compound of structure (I) in vivo when such prodrug is administered to a patient. Prodrugs are generally prepared by modifying functional groups in a way such that the modification is cleaved, either by routine manipulation or in vivo, yielding the parent compound. Prodrugs include, for example, compounds of this invention wherein hydroxy, amine or sulfhydryl groups are bonded to any group that, when administered to a patient, cleaves to form the hydroxy, amine or sulfhydryl groups. Thus, representative examples of prodrugs include (but are not limited to) acetate, formate and benzoate derivatives of alcohol and amine functional groups of the compounds of structure (I). Further, in the case of a carboxylic acid (—COOH), esters may be employed, such as methyl esters, ethyl esters, and the like.

With regard to stereoisomers, the compounds of structure (I) may have chiral centers and may occur as racemates, racemic mixtures and as individual enantiomers or diastereomers. All such isomeric forms are included within the present invention, including mixtures thereof. Furthermore, some of the crystalline forms of the compounds of structure (I) may exist as polymorphs, which are included in the present invention. In addition, some of the compounds of structure (I) may also form solvates with water or other organic solvents. Such solvates are similarly included within the scope of this invention.

A modulating agent as described herein may, but need not, be linked to one or more additional molecules. In particular, as discussed below, it may be beneficial for certain applications to link multiple modulating agents (which may, but need not, be identical) to a support molecule (e.g., keyhole limpet hemocyanin) or a solid support, such as a polymeric matrix (which may be formulated as a membrane or microstructure, such as an ultra thin film), a container surface (e.g., the surface of a tissue culture plate or the interior surface of a bioreactor), or a bead or other particle, which may be prepared from a variety of materials including glass, plastic or ceramics. For certain applications, biodegradable support materials are preferred, such as cellulose and derivatives thereof, collagen, spider silk or any of a variety of polyesters (e.g., those derived from hydroxy acids and/or lactones) or sutures (see U.S. Pat. No. 5,245,012). Within certain embodiments, modulating agents and molecules comprising compounds of formula (I) may be attached to a support such as a polymeric matrix, preferably in an alternating pattern.

Suitable methods for linking a modulating agent to a support material will depend upon the composition of the support and the intended use, and will be readily apparent to those of ordinary skill in the art. Attachment may generally be achieved through noncovalent association, such as adsorption or affinity or, preferably, via covalent attachment (which may be a direct linkage between a modulating agent and functional groups on the support, or may be a linkage by way of a cross-linking agent or linker). Attachment of a modulating agent by adsorption may be achieved by contact, in a suitable buffer, with a solid support for a suitable amount of time. The contact time varies with temperature, but is generally between about 5 seconds and 1 day, and typically between about 10 seconds and 1 hour.

Covalent attachment of a modulating agent to a molecule or solid support may generally be achieved by first reacting the support material with a bifunctional reagent that will also react with a functional group, such as a hydroxyl, thiol, carboxyl, ketone or amino group, on the modulating agent. For example, a modulating agent may be bound to an appropriate polymeric support or coating using benzoquinone, by condensation of an aldehyde group on the support with an amine and an active hydrogen on the modulating agent or by condensation of an amino group on the support with a carboxylic acid on the modulating agent. A preferred method of generating a linkage is via amino groups using glutaraldehyde. A modulating agent may be linked to cellulose via ester linkages. Similarly, amide linkages may be suitable for linkage to other molecules such as keyhole limpet hemocyanin or other support materials. Multiple modulating agents and/or molecules comprising compounds of formula (I) may be attached, for example, by random coupling, in which equimolar amounts of such molecules are mixed with a matrix support and allowed to couple at random.

Although modulating agents as described herein may preferentially bind to specific tissues or cells, and thus may be sufficient to target a desired site in vivo, it may be beneficial for certain applications to include an additional targeting agent. Accordingly, a targeting agent may also, or alternatively, be linked to a modulating agent to facilitate targeting to one or more specific tissues. As used herein, a “targeting agent,” may be any substance (such as a compound or cell) that, when linked to a modulating agent enhances the transport of the modulating agent to a target tissue, thereby increasing the local concentration of the modulating agent. Targeting agents include antibodies or fragments thereof, receptors, ligands and other molecules that bind to cells of, or in the vicinity of, the target tissue. Known targeting agents include serum hormones, antibodies against cell surface antigens, lectins, adhesion molecules, tumor cell surface binding ligands, steroids, cholesterol, lymphokines, fibrinolytic enzymes and those drugs and proteins that bind to a desired target site. Among the many monoclonal antibodies that may serve as targeting agents are anti-TAC, or other interleukin-2 receptor antibodies; 9.2.27 and NR-ML-05, reactive with the 250 kilodalton human melanoma-associated proteoglycan; and NR-LU-10, reactive with a pancarcinoma glycoprotein. An antibody targeting agent may be an intact (whole) molecule, a fragment thereof, or a functional equivalent thereof. Examples of antibody fragments are F(ab′)2, -Fab′, Fab and F[v] fragments, which may be produced by conventional methods or by genetic or protein engineering. Linkage is generally covalent and may be achieved by, for example, direct condensation or other reactions, or by way of bi- or multi-functional linkers. Within other embodiments, it may also be possible to target a polynucleotide encoding a modulating agent to a target tissue, thereby increasing the local concentration of modulating agent. Such targeting may be achieved using well known techniques, including retroviral and adenoviral infection.

For certain embodiments, it may be beneficial to also, or alternatively, link a drug to a modulating agent. As used herein, the term “drug” refers to any bioactive agent intended for administration to a mammal to prevent or treat a disease or other undesirable condition. Drugs include hormones, growth factors, proteins, peptides and other compounds. The use of certain specific drugs within the context of the present invention is discussed below.

Within certain aspects of the present invention, one or more modulating agents as described herein may be present within a pharmaceutical composition. A pharmaceutical composition comprises one or more modulating agents in combination with one or more pharmaceutically or physiologically acceptable carriers, diluents or excipients. Such compositions may comprise buffers (e.g., neutral buffered saline or phosphate buffered saline), carbohydrates (e.g., glucose, mannose, sucrose or dextrans), mannitol, proteins, polypeptides or amino acids such as glycine, antioxidants, chelating agents such as EDTA or glutathione, adjuvants (e.g., aluminum hydroxide) and/or preservatives. Within yet other embodiments, compositions of the present invention may be formulated as a lyophilizate. A modulating agent (alone or in combination with a targeting agent and/or drug) may, but need not, be encapsulated within liposomes using well known technology. Compositions of the present invention may be formulated for any appropriate manner of administration, including for example, topical, oral, nasal, intravenous, intracranial, intraperitoneal, subcutaneous, or intramuscular administration. For certain topical applications, formulation as a cream or lotion, using well known components, is preferred.

For certain embodiments, as discussed below, a pharmaceutical composition may further comprise a modulator of cell adhesion that is mediated by one or more molecules other than cadherins. Such compositions are particularly useful for situations in which it is desirable to inhibit cell adhesion mediated by multiple cell-adhesion molecules, such as other members of the cadherin gene superfamily that are not classical cadherins (e.g., Dsg and Dsc); claudins; integrins; members of the immunoglobulin supergene family, such as N-CAM; and other uncategorized transmembrane proteins, such as occludin, as well as extracellular matrix proteins such as laminin, fibronectin, collagens, vitronectin, entactin and tenascin.

A pharmaceutical composition may also contain one or more drugs, as further discussed below, which may be linked to a modulating agent or may be free within the composition. Virtually any drug may be administered in combination with a modulating agent as described herein, for a variety of purposes as described below. Examples of types of drugs that may be administered with a modulating agent include analgesics, anesthetics, antianginals, antifungals, antibiotics, anticancer drugs (e.g., taxol or mitomycin C), antiinflammatories (e.g., ibuprofen and indomethacin), anthelmintics, antidepressants, antidotes, antiemetics, antihistamines, antihypertensives, antimalarials, antimicrotubule agents (e.g., colchicine or vinca alkaloids), antimigraine agents, antimicrobials, antiphsychotics, antipyretics, antiseptics, anti-signaling agents (e.g., protein kinase C inhibitors or inhibitors of intracellular calcium mobilization), antiarthritics, antithrombin agents, antituberculotics, antitussives, antivirals, appetite suppressants, cardioactive drugs, chemical dependency drugs, cathartics, chemotherapeutic agents, coronary, cerebral or peripheral vasodilators, contraceptive agents, depressants, diuretics, expectorants, growth factors, hormonal agents, hypnotics, immunosuppression agents, narcotic antagonists, parasympathomimetics, sedatives, stimulants, sympathomimetics, toxins (e.g., cholera toxin), tranquilizers and urinary antiinfectives.

For imaging purposes, any of a variety of diagnostic agents may be incorporated into a pharmaceutical composition, either linked to a modulating agent or free within the composition. Diagnostic agents include any substance administered to illuminate a physiological function within a patient, while leaving other physiological functions generally unaffected. Diagnostic agents include metals, radioactive isotopes and radioopaque agents (e.g., gallium, technetium, indium, strontium, iodine, barium, bromine and phosphorus-containing compounds), radiolucent agents, contrast agents, dyes (e.g., fluorescent dyes and chromophores) and enzymes that catalyze a calorimetric or fluorometric reaction. In general, such agents may be attached using a variety of techniques as described above, and may be present in any orientation.

The compositions described herein may be administered as part of a sustained release formulation (i.e., a formulation such as a capsule or sponge that effects a slow release of modulating agent following administration). Such formulations may generally be prepared using well known technology and administered by, for example, oral, rectal or subcutaneous implantation, or by implantation at the desired target site. Sustained-release formulations may contain a modulating agent dispersed in a carrier matrix and/or contained within a reservoir surrounded by a rate controlling membrane (see, e.g., European Patent Application 710,491A). Carriers for use within such formulations are biocompatible, and may also be biodegradable; preferably the formulation provides a relatively constant level of modulating agent release. The amount of modulating agent contained within a sustained release formulation depends upon the site of implantation, the rate and expected duration of release and the nature of the condition to be treated or prevented.

Pharmaceutical compositions of the present invention may be administered in a manner appropriate to the disease to be treated (or prevented). Appropriate dosages and the duration and frequency of administration will be determined by such factors as the condition of the patient, the type and severity of the patient's disease and the method of administration. In general, an appropriate dosage and treatment regimen provides the modulating agent(s) in an amount sufficient to provide therapeutic and/or prophylactic benefit. Within particularly preferred embodiments of the invention, a modulating agent or pharmaceutical composition as described herein may be administered at a dosage ranging from 0.001 to 50 mg/kg body weight, preferably from 0.1 to 20 mg/kg, on a regimen of single or multiple daily doses. For topical administration, a cream typically comprises an amount of modulating agent ranging from 0.00001% to 1%, preferably 0.0001% to 0.2%, and more preferably from 0.0001% to 0.002%. Fluid compositions typically contain about 10 ng/ml to 5 mg/ml, preferably from about 10 μg to 2 mg/mL of compounds of formula (I). Appropriate dosages may generally be determined using experimental models and/or clinical trials. In general, the use of the minimum dosage that is sufficient to provide effective therapy is preferred. Patients may generally be monitored for therapeutic effectiveness using assays suitable for the condition being treated or prevented, which will be familiar to those of ordinary skill in the art.

### Modulating Agent Methods of Use

In general, the modulating agents and compositions described herein may be used for modulating the adhesion of classical cadherin-expressing cells (i.e., cells that express one or more of E-cadherin, N-cadherin, P-cadherin, R-cadherin and/or other cadherin(s) containing the HAV sequence, including as yet undiscovered classical cadherins) in vitro and/or in vivo. To modulate classical cadherin-mediated cell adhesion, a cadherin-expressing cell is contacted with a modulating agent either in vivo or in vitro. As noted above, modulating agents for purposes that involve the disruption of cadherin-mediated cell adhesion may comprise a single compound of formula (I) or multiple multiple compounds of formula (I) in close proximity. When it is desirable to also disrupt cell adhesion mediated by other adhesion molecules, a composition comprising the modulating agent may additionally comprise one or more additional modulating agents bound by such adhesion molecules (and/or antibodies or fragments thereof that bind such sequences), preferably separated by linkers. As noted above, such linkers may or may not comprise one or more amino acids.

Certain methods involving the disruption of cell adhesion as described herein have an advantage over prior techniques in that they permit the passage of molecules that are large and/or charged across barriers of cadherin-expressing cells. As discussed in greater detail below, modulating agents as described herein may also be used to disrupt or enhance cell adhesion in a variety of other contexts. Within the methods described herein, one or more modulating agents may generally be administered alone, or within a pharmaceutical composition. In each specific method described herein, as noted above, a targeting agent may be employed to increase the local concentration of modulating agent at the target site.

In one such aspect, the present invention provides methods for reducing unwanted cellular adhesion by administering a modulating agent as described herein. Unwanted cellular adhesion can occur between tumor cells, between tumor cells and normal cells or between normal cells as a result of surgery, injury, chemotherapy, disease, inflammation or other condition jeopardizing cell viability or function. Preferred modulating agents for use within such methods comprise a single compound of formula (I). Alternatively, a separate modulator of integrin, occludin-, OB-cadherin-, dsc- and/or dsg-mediated cell adhesion may be administered in conjunction with the modulating agent(s), either within the same pharmaceutical composition or separately. Topical administration of the modulating agent(s) is generally preferred, but other means may also be employed. Preferably, a fluid composition for topical administration (comprising, for example, physiological saline) comprises an amount of a compound of formula (I) as described above, and more preferably an amount ranging from 10 μg/mL to 1 mg/mL. Creams may generally be formulated as described above. Topical administration in the surgical field may be given once at the end of surgery by irrigation of the wound, as an intermittent or continuous irrigation with use of surgical drains in the post operative period, or by the use of drains specifically inserted in an area of inflammation, injury or disease in cases where surgery does not need to be performed. Alternatively, parenteral or transcutaneous administration may be used to achieve similar results.

In another aspect, methods are provided for enhancing the delivery of a drug through the skin of a mammal. Transdermal delivery of drugs is a convenient and non-invasive method that can be used to maintain relatively constant blood levels of a drug. In general, to facilitate drug delivery via the skin, it is necessary to perturb adhesion between the epithelial cells (keratinocytes) and the endothelial cells of the microvasculature. Using currently available techniques, only small, uncharged molecules may be delivered across skin in vivo. The methods described herein are not subject to the same degree of limitation. Accordingly, a wide variety of drugs may be transported across the epithelial and endothelial cell layers of skin, for systemic or topical administration. Such drugs may be delivered to melanomas or may enter the blood stream of the mammal for delivery to other sites within the body.

To enhance the delivery of a drug through the skin, a modulating agent as described herein and a drug are contacted with the skin surface. Preferred modulating agents for use within such methods comprise a single compound of formula (I). Multifunctional modulating agents comprising such a compound of formula (I) linked to one or more other modulating agent may also be used to disrupt epithelial cell adhesion. Alternatively, a separate modulator of non-classical cadherin-mediated cell adhesion may be administered in conjunction with the modulating agent(s), either within the same pharmaceutical composition or separately.

Contact may be achieved by direct application of the modulating agent, generally within a composition formulated as a cream or gel, or using any of a variety of skin contact devices for transdermal application (such as those described in European Patent Application No. 566,816 A; U.S. Pat. No. 5,613,958; U.S. Pat. No. 5,505,956). A skin patch provides a convenient method of administration (particularly for slow-release formulations). Such patches may contain a reservoir of modulating agent and drug separated from the skin by a membrane through which the drug diffuses. Within other patch designs, the modulating agent and drug may be dissolved or suspended in a polymer or adhesive matrix that is then placed in direct contact with the patient's skin. The modulating agent and drug may then diffuse from the matrix into the skin. Modulating agent(s) and drug(s) may be contained within the same composition or skin patch, or may be separately administered, although administration at the same time and site is preferred. In general, the amount of modulating agent administered via the skin varies with the nature of the condition to be treated or prevented, but may vary as described above. Such levels may be achieved by appropriate adjustments to the device used, or by applying a cream formulated as described above. Transfer of the drug across the skin and to the target tissue may be predicted based on in vitro studies using, for example, a Franz cell apparatus, and evaluated in vivo by appropriate means that will be apparent to those of ordinary skill in the art. As an example, monitoring of the serum level of the administered drug over time provides a convenient measure of the drug transfer across the skin.

Transdermal drug delivery as described herein is particularly useful in situations in which a constant rate of drug delivery is desired, to avoid fluctuating blood levels of a drug. For example, morphine is an analgesic commonly used immediately following surgery. When given intermittently in a parenteral form (intramuscular, intravenous), the patient usually feels sleepy during the first hour, is well during the next 2 hours and is in pain during the last hour because the blood level goes up quickly after the injection and goes down below the desirable level before the 4 hour interval prescribed for re-injection is reached. Transdermal administration as described herein permits the maintenance of constant levels for long periods of time (e.g., days), which allows adequate pain control and mental alertness at the same time. Insulin provides another such example. Many diabetic patients need to maintain a constant baseline level of insulin which is different from their needs at the time of meals. The baseline level may be maintained using transdermal administration of insulin, as described herein. Antibiotics may also be administered at a constant rate, maintaining adequate bactericidal blood levels, while avoiding the high levels that are often responsible for the toxicity (e.g., levels of gentamycin that are too high typically result in renal toxicity).

Drug delivery by the methods of the present invention also provide a more convenient method of drug administration. For example, it is often particularly difficult to administer parenteral drugs to newborns and infants because of the difficulty associated with finding veins of acceptable caliber to catheterize. However, newborns and infants often have a relatively large skin surface as compared to adults. Transdermal drug delivery permits easier management of such patients and allows certain types of care that can presently be given only in hospitals to be given at home. Other patients who typically have similar difficulties with venous catheterization are patients undergoing chemotherapy or patients on dialysis. In addition, for patients undergoing prolonged therapy, transdermal administration as described herein is more convenient than parenteral administration.

Transdermal administration as described herein also allows the gastrointestinal tract to be bypassed in situations where parenteral uses would not be practical. For example, there is a growing need for methods suitable for administration of therapeutic small peptides and proteins, which are typically digested within the gastrointestinal tract. The methods described herein permit administration of such compounds and allow easy administration over long periods of time. Patients who have problems with absorption through their gastrointestinal tract because of prolonged ileus or specific gastrointestinal diseases limiting drug absorption may also benefit from drugs formulated for transdermal application as described herein.

Further, there are many clinical situations where it is difficult to maintain compliance. For example, patients with mental problems (e.g., patients with Alzheimer's disease or psychosis) are easier to manage if a constant delivery rate of drug is provided without having to rely on their ability to take their medication at specific times of the day. Also patients who simply forget to take their drugs as prescribed are less likely to do so if they merely have to put on a skin patch periodically (e.g., every 3 days). Patients with diseases that are without symptoms, like patients with hypertension, are especially at risk of forgetting to take their medication as prescribed.

For patients taking multiple drugs, devices for transdermal application such as skin patches may be formulated with combinations of drugs that are frequently used together. For example, many heart failure patients are given digoxin in combination with furosemide. The combination of both drugs into a single skin patch facilitates administration, reduces the risk of errors (taking the correct pills at the appropriate time is often confusing to older people), reduces the psychological strain of taking “so many pills,” reduces skipped dosage because of irregular activities and improves compliance.

The methods described herein are particularly applicable to humans, but also have a variety of veterinary uses, such as the administration of growth factors or hormones (e.g., for fertility control) to an animal.

As noted above, a wide variety of drugs may be administered according to the methods provided herein. Some examples of drug categories that may be administered transdermally include anti-inflammatory drugs (e.g., in arthritis and in other condition) such as all NSAID, indomethacin, prednisone, etc.; analgesics (especially when oral absorption is not possible, such as after surgery, and when parenteral administration is not convenient or desirable), including morphine, codeine, Demerol, acetaminophen and combinations of these (e.g., codeine plus acetaminophen); antibiotics such as Vancomycin (which is not absorbed by the GI tract and is frequently given intravenously) or a combination of INH and Rifampicin (e.g., for tuberculosis); anticoagulants such as heparin (which is not well absorbed by the GI tract and is generally given parenterally, resulting in fluctuation in the blood levels with an increased risk of bleeding at high levels and risks of inefficacy at lower levels) and Warfarin (which is absorbed by the GI tract but cannot be administered immediately after abdominal surgery because of the normal ileus following the procedure); antidepressants (e.g., in situations where compliance is an issue as in Alzheimer's disease or when maintaining stable blood levels results in a significant reduction of anti-cholinergic side effects and better tolerance by patients), such as amitriptylin, imipramin, prozac, etc.; antihypertensive drugs (e.g., to improve compliance and reduce side effects associated with fluctuating blood levels), such as diuretics and beta-blockers (which can be administered by the same patch; e.g., furosemide and propanolol); antipsychotics (e.g., to facilitate compliance and make it easier for care giver and family members to make sure that the drug is received), such as haloperidol and chlorpromazine; and anxiolytics or sedatives (e.g., to avoid the reduction of alertness related to high blood levels after oral administration and allow a continual benefit throughout the day by maintaining therapeutic levels constant).

Numerous other drugs may be administered as described herein, including naturally occurring and synthetic hormones, growth factors, proteins and peptides. For example, insulin and human growth hormone, growth factors like erythropoietin, interleukins and interferons may be delivered via the skin.

Kits for administering a drug via the skin of a mammal are also provided within the present invention. Such kits generally comprise a device for transdermal application (i.e., skin patch) in combination with, or impregnated with, one or more modulating agents. A drug may additionally be included within such kits.

Within a related embodiment, the use of modulating agents as described herein to increase skin permeability may also facilitate sampling of the blood compartment by passive diffusion, permitting detection and/or measurement of the levels of specific molecules circulating in the blood. For example, application of one or more modulating agents to the skin, via a skin patch as described herein, permits the patch to function like a sponge to accumulate a small quantity of fluid containing a representative sample of the serum. The patch is then removed after a specified amount of time and analyzed by suitable techniques for the compound of interest (e.g., a medication, hormone, growth factor, metabolite or marker). Alternatively, a patch may be impregnated with reagents to permit a color change if a specific substance (e.g., an enzyme) is detected. Substances that can be detected in this manner include, but are not limited to, illegal drugs such as cocaine, HIV enzymes, glucose and PSA. This technology is of particular benefit for home testing kits.

Within a further aspect, methods are provided for enhancing delivery of a drug to a tumor in a mammal, comprising administering a modulating agent in combination with a drug to a tumor-bearing mammal. Modulating agents for use within such methods include compounds of formula (I).

In one particularly preferred embodiment, a modulating agent is capable of disrupting cell adhesion mediated by multiple adhesion molecules. Such agents serve as multifunctional disrupters of cell adhesion. Alternatively, a separate modulator of non-classical cadherin-mediated cell adhesion may be administered in conjunction with the modulating agent(s), either within the same pharmaceutical composition or separately. Antibodies or Fab fragments directed against a cadherin CAR sequence and/or an occludin CAR sequence may also be employed, either incorporated into a modulating agent or within a separate modulator that is administered concurrently.

Preferably, the modulating agent and the drug are formulated within the same composition or drug delivery device prior to administration. In general, a modulating agent may enhance drug delivery to any tumor, and the method of administration may be chosen based on the type of target tumor. For example, injection or topical administration as described above may be preferred for melanomas and other accessible tumors (e.g., metastases from primary ovarian tumors may be treated by flushing the peritoneal cavity with the composition). Other tumors (e.g., bladder tumors) may be treated by injection of the modulating agent and the drug (such as mitomycin C) into the site of the tumor. In other instances, the composition may be administered systemically, and targeted to the tumor using any of a variety of specific targeting agents. Suitable drugs may be identified by those of ordinary skill in the art based upon the type of cancer to be treated (e.g., mitomycin C for bladder cancer). In general, the amount of modulating agent administered varies with the method of administration and the nature of the tumor, within the typical ranges provided above, preferably ranging from about 1 μg/mL to about 2 mg/mL, and more preferably from about 10 μg/mL to 100 μg/mL. Transfer of the drug to the target tumor may be evaluated by appropriate means that will be apparent to those of ordinary skill in the art, such as a reduction in tumor size. Drugs may also be labeled (e.g., using radionuclides) to permit direct observation of transfer to the target tumor using standard imaging techniques.

Within a related aspect, the present invention provides methods for inhibiting the development of a cancer (i.e., for treating or preventing cancer and/or inhibiting metastasis) in a mammal. Cancer tumors are solid masses of cells, growing out of control, which require nourishment via blood vessels. The formation of new capillaries is a prerequisite for tumor growth and the emergence of metastases. Administration of a modulating agent as described herein may disrupt the growth of such blood vessels, thereby providing effective therapy for the cancer and/or inhibiting metastasis. Modulating agents comprising compounds of formula (I) may also be used to treat leukemias. Preferred modulating agents for use within such methods include those that disrupt N-cadherin mediated cell adhesion, such as agents that comprise a compound of formula (I). Alternatively, a separate modulator of integrin-OB-cadherin-, dsc-, dsg-, claudin- and/or occludin-mediated cell adhesion may be administered in conjunction with the modulating agent(s), either within the same pharmaceutical composition or separately.

The compounds and compositions of the invention can be used to treat essentially any cancer wherein administration thereto provides at least some clinical benefit. In certain embodiments, the cancer is a cancer that expresses a classical cadherin protein. Illustrative cancers include lung cancer, NSCLC (non small cell lung cancer), bone cancer, pancreatic cancer, skin cancer, cancer of the head and neck, cutaneous or intraocular melanoma, uterine cancer, ovarian cancer, colo-rectal cancer, cancer of the anal region, stomach cancer, colon cancer, breast cancer, gynecologic tumors, Hodgkin's Disease, hepatocellular cancer, cancer of the esophagus, cancer of the small intestine, cancer of the endocrine system (e.g., cancer of the thyroid, pancreas, parathyroid or adrenal glands), sarcomas of soft tissues, cancer of the urethra, cancer of the penis, prostate cancer, chronic or acute leukemia, solid tumors of childhood, hypereosinophilia, lymphocytic lymphomas, cancer of the bladder, cancer of the kidney or ureter (e.g., renal cell carcinoma, carcinoma of the renal pelvis), pediatric malignancy, neoplasms of the central nervous system (e.g., primary CNS lymphoma, spinal axis tumors, medulloblastoma, brain stem gliomas or pituitary adenomas), Barrett's esophagus (pre-malignant syndrome), neoplastic cutaneous disease, etc.

A modulating agent may be administered alone (e.g., via the skin) or within a pharmaceutical composition. For melanomas and certain other accessible tumors, injection or topical administration as described above may be preferred. For ovarian cancers, flushing the peritoneal cavity with a composition comprising one or more modulating agents may prevent metastasis of ovarian tumor cells. Other tumors (e.g., bladder tumors, bronchial tumors or tracheal tumors) may be treated by injection of the modulating agent into the cavity. In other instances, the composition may be administered systemically, and targeted to the tumor using any of a variety of specific targeting agents, as described above. In general, the amount of modulating agent administered varies depending upon the method of administration and the nature of the cancer, but may vary within the ranges identified above. The effectiveness of the cancer treatment or inhibition of metastasis may be evaluated using well known clinical observations such as the level of serum markers (e.g., CEA or PSA).

Within a further related aspect, a modulating agent may be used to inhibit angiogenesis (i.e., the growth of blood vessels from pre-existing blood vessels) in a mammal. In general, inhibition of angiogenesis may be beneficial in patients afflicted with diseases such as cancer or arthritis. Preferred modulating agents for use within such methods comprise a single compound of formula (I). Alternatively, a separate modulator of integrin- and/or occludin-mediated cell adhesion may be administered in conjunction with the modulating agent(s), either within the same pharmaceutical composition or separately.

The effect of a particular modulating agent on angiogenesis may generally be determined by evaluating the effect of the agent on blood vessel formation. Such a determination may generally be performed, for example, using a chick chorioallantoic membrane assay (Iruela-Arispe et al., Molecular Biology of the Cell 6:327-343, 1995). Briefly, a modulating agent may be embedded in a mesh composed of vitrogen at one or more concentrations (e.g., ranging from about 1 to 100 μg/mesh). The mesh(es) may then be applied to chick chorioallantoic membranes. After 24 hours, the effect of the agent may be determined using computer assisted morphometric analysis. A modulating agent should inhibit angiogenesis by at least 25% at a concentration of 33 μg/mesh.

The addition of a targeting agent may be beneficial, particularly when the administration is systemic. Suitable modes of administration and dosages depend upon the condition to be prevented or treated but, in general, administration by injection is appropriate. Dosages may vary as described above. The effectiveness of the inhibition may be evaluated grossly by assessing the inability of the tumor to maintain growth and microscopically by an absence of nerves at the periphery of the tumor.

In yet another related aspect, the present invention provides methods for inducing apoptosis in a cadherin-expressing cell. In general, patients afflicted with cancer may benefit from such treatment. Preferred modulating agents for use within such methods comprise a single compound of formula (I). Alternatively, a separate modulator of cell adhesion mediated by an adhesion molecule that is not a cadherin may be administered in conjunction with the modulating agent(s), either within the same pharmaceutical composition or separately. Administration may be topical, via injection or by other means, and the addition of a targeting agent may be beneficial, particularly when the administration is systemic. Suitable modes of administration and dosages depend upon the location and nature of the cells for which induction of apoptosis is desired but, in general, dosages may vary as described above. A biopsy may be performed to evaluate the level of induction of apoptosis.

The present invention also provides methods for enhancing drug delivery to the central nervous system of a mammal. The blood/brain barrier is largely impermeable to most neuroactive agents, and delivery of drugs to the brain of a mammal often requires invasive procedures. Using a modulating agent as described herein, however, delivery may be by, for example, systemic administration of a composition comprising a compound of formula (I), injection of a composition comprising a compound of formula (I) (alone or in combination with a drug and/or targeting agent) into the carotid artery or application of a skin patch comprising a modulating agent to the head of the patient. Alternatively, a separate modulator of occludin-mediated cell adhesion may be administered in conjunction with the modulating agent(s), either within the same pharmaceutical composition or separately. Modulating agents may further comprise antibodies or Fab fragments directed against the N-cadherin CAR sequence FHLRAHAVDINGNQV-NH₂. Fab fragments directed against the occludin CAR sequence region GVNPTAQSSGSLYGSQIYALCNQFYTPAATGLYVDQYLYHYCVVDPQE may also be employed, either incorporated into the modulating agent or administered concurrently as a separate modulator.

In general, the amount of modulating agent administered varies with the method of administration and the nature of the condition to be treated or prevented, but typically varies as described above. Transfer of the drug to the central nervous system may be evaluated by appropriate means that will be apparent to those of ordinary skill in the art, such as magnetic resonance imaging (MRI) or PET scan (positron emitted tomography).

In still further aspects, the present invention provides methods for enhancing adhesion of cadherin-expressing cells. Within certain embodiments, a modulating agent may be linked to a support molecule or to a solid support as described above, resulting in a matrix that comprises multiple modulating agents. Within one such embodiment, the support is a polymeric matrix to which other modulating agents are attached (e.g., modulating agents and molecules comprising RGD, LYHY or a CAR sequence for OB-cadherin, a desmoglein, a desmocollin or claudin, may be attached to the same matrix, preferably in an alternating pattern). Such matrices may be used in contexts in which it is desirable to enhance adhesion mediated by multiple cell adhesion molecules. Alternatively, the modulating agent itself may comprise multiple compounds of formula (I), separated by linkers as described above. Either way, the modulating agent(s) function as a “biological glue” to bind multiple cadherin-expressing cells within a variety of contexts.

Within one embodiment, such modulating agents may be used to enhance wound healing and/or reduce scar tissue in a mammal. Preferred modulating agents for use within such methods comprise a single compound of formula (I). Modulating agents that are linked to a biocompatible and biodegradable matrix such as cellulose or collagen are particularly preferred. For use within such methods, a modulating agent should have a free amino or hydroxyl group. Alternatively, one or more separate modulators of integrin-, Dsc-, Dsg-, claudin-, OB-cadherin- and/or occludin-mediated cell adhesion may be administered in conjunction with the modulating agent(s), either within the same pharmaceutical composition or separately.

The modulating agents are generally administered topically to the wound, where they may facilitate closure of the wound and may augment, or even replace, stitches. Similarly, administration of matrix-linked modulating agents may facilitate cell adhesion in foreign tissue implants (e.g., skin grafting and prosthetic implants) and may prolong the duration and usefulness of collagen injection. In general, the amount of matrix-linked compound of formula (I) administered to a wound, graft or implant site varies with the severity of the wound and/or the nature of the wound, graft, or implant, but may vary as discussed above.

Within another embodiment, one or more modulating agents may be linked to the interior surface of a tissue culture plate or other cell culture support, such as for use in a bioreactor. Such linkage may be performed by any suitable technique, as described above. Modulating agents linked in this fashion may generally be used to immobilize cadherin-expressing cells. For example, dishes or plates coated with one or more modulating agents may be used to immobilize cadherin-expressing cells within a variety of assays and screens. Within bioreactors (i.e., systems for larger scale production of cells or organoids), modulating agents may generally be used to improve cell attachment and stabilize cell growth. Modulating agents may also be used within bioreactors to support the formation and function of highly differentiated organoids derived, for example, from dispersed populations of fetal mammalian cells. Bioreactors containing compound(s) of formula (I) may also be used to facilitate the production of specific proteins.

Modulating agents as described herein may be used within a variety of bioreactor configurations. In general, a bioreactor is designed with an interior surface area sufficient to support larger numbers of adherent cells. This surface area can be provided using membranes, tubes, microtiter wells, columns, hollow fibers, roller bottles, plates, dishes, beads or a combination thereof. A bioreactor may be compartmentalized. The support material within a bioreactor may be any suitable material known in the art; preferably, the support material does not dissolve or swell in water. Preferred support materials include, but are not limited to, synthetic polymers such as acrylics, vinyls, polyethylene, polypropylene, polytetrafluoroethylene, nylons, polyurethanes, polyamides, polysulfones and poly(ethylene terephthalate); ceramics; glass and silica.

Modulating agents may also be used, within other aspects of the present invention, to enhance and/or direct neurological growth. In one aspect, neurite outgrowth may be enhanced and/or directed by contacting a neuron with one or more modulating agents. Preferred modulating agents for use within such methods are linked to a polymeric matrix or other support, and comprise a compound of formula (I). Modulating agents comprising antibodies, or fragments thereof, may be used within this aspect of the present invention without the use of linkers or support materials. The method of achieving contact and the amount of modulating agent used will depend upon the location of the neuron and the extent and nature of the outgrowth desired. For example, a neuron may be contacted (e.g., via implantation) with modulating agent(s) linked to a support material such as a suture, fiber nerve guide or other prosthetic device such that the neurite outgrowth is directed along the support material. Alternatively, a tubular nerve guide may be employed, in which the lumen of the nerve guide contains a composition comprising the modulating agent(s). In vivo, such nerve guides or other supported modulating agents may be implanted using well known techniques to, for example, facilitate the growth of severed neuronal connections and/or to treat spinal cord injuries. It will be apparent to those of ordinary skill in the art that the structure and composition of the support should be appropriate for the particular injury being treated. In vitro, a polymeric matrix may similarly be used to direct the growth of neurons onto patterned surfaces as described, for example, in U.S. Pat. No. 5,510,628.

Within another such aspect, one or more modulating agents may be used for therapy of a demyelinating neurological disease in a mammal. There are a number of demyelinating diseases, such as multiple sclerosis, characterized by oligodendrocyte death. It has been found, within the context of the present invention, that Schwann cell migration on astrocytes is inhibited by N-cadherin. Modulating agents that disrupt N-cadherin mediated cell adhesion as described herein may be implanted into the central nervous system with cells capable of replenishing an oligodendrocyte population, such as Schwann cells, oligodendrocytes or oligodendrocyte precursor cells. Such therapy may facilitate of the cell capable of replenishing an oligodendrocyte population and permit the practice of Schwann cell or oligodendrocyte replacement therapy.

Multiple sclerosis patients suitable for treatment may be identified by criteria that establish a diagnosis of clinically definite or clinically probable MS (see Poser et al., Ann. Neurol. 13:227, 1983). Candidate patients for preventive therapy may be identified by the presence of genetic factors, such as HLA-type DR2a and DR2b, or by the presence of early disease of the relapsing remitting type.

Schwann cell grafts may be implanted directly into the brain along with the modulating agent(s) using standard techniques. Preferred modulating agents for use within such methods comprise a compound of formula (I). Modulating agents comprising antibodies, or fragments thereof, may also be used within this aspect of the present invention. Preferred antibody modulating agents include Fab fragments directed against the N-cadherin CAR sequence FHLRAHAVDINGNQV-NH₂. Suitable amounts of compounds of formula (I) generally range as described above, preferably from about 10 μg/mL to about 1 mg/mL.

Alternatively, a modulating agent may be implanted with oligodendrocyte progenitor cells (OPs) derived from donors not afflicted with the demyelinating disease. The myelinating cell of the CNS is the oligodendrocyte. Although mature oligodendrocytes and immature cells of the oligodendrocyte lineage, such as the oligodendrocyte type 2 astrocyte progenitor, have been used for transplantation, OPs are more widely used. OPs are highly motile and are able to migrate from transplant sites to lesioned areas where they differentiate into mature myelin-forming oligodendrocytes and contribute to repair of demyelinated axons (see e.g., Groves et al., Nature 362:453-55, 1993; Baron-Van Evercooren et al., Glia 16:147-64, 1996). OPs can be isolated using routine techniques known in the art (see e.g., Milner and French-Constant, Development 120:3497-3506, 1994), from many regions of the CNS including brain, cerebellum, spinal cord, optic nerve and olfactory bulb. Substantially greater yields of OP's are obtained from embryonic or neonatal rather than adult tissue. OPs may be isolated from human embryonic spinal cord and cultures of neurospheres established. Human fetal tissue is a potential valuable and renewable source of donor OP's for future, long range transplantation therapies of demyelinating diseases such as MS.

OPs can be expanded in vitro if cultured as “homotypic aggregates” or “spheres” (Avellana-Adalid et al, J. Neurosci. Res. 45:558-70, 1996). Spheres (sometimes called “oligospheres” or “neurospheres”) are formed when OPs are grown in suspension in the presence of growth factors such as PDGF and FGF. OPs can be harvested from spheres by mechanical dissociation and used for subsequent transplantation or establishment of new spheres in culture. Alternatively, the spheres themselves may be transplanted, providing a “focal reservoir” of OPs (Avellana-Adalid et al, J. Neurosci. Res. 45:558-70, 1996).

An alternative source of OP may be spheres derived from CNS stem cells. Recently, Reynolds and Weiss, Dev. Biol. 165:1-13, 1996 have described spheres formed from EGF-responsive cells derived from embryonic neuroepithelium, which appear to retain the pluripotentiality exhibited by neuroepithelium in vivo. Cells dissociated from these spheres are able to differentiate into neurons, oligodendrocytes and astrocytes when plated on adhesive substrates in the absence of EGF, suggesting that EGF-responsive cells derived from undifferentiated embryonic neuroepithelium may represent CNS stem cells (Reynolds and Weiss, Dev. Biol. 165:1-13, 1996). Spheres derived from CNS stem cells provide an alternative source of OP which may be manipulated in vitro for transplantation in vivo. Spheres composed of CNS stem cells may further provide a microenvironment conducive to increased survival, migration, and differentiation of the OPs in vivo.

The use of neurospheres for the treatment of MS may be facilitated by modulating agents that enhance cell migration from the spheres. In the absence of modulating agent, the cells within the spheres adhere tightly to one another and migration out of the spheres is hindered. Modulating agents that disrupt N-cadherin mediated cell adhesion as described herein, when injected with neurospheres into the central nervous system, may improve cell migration and increase the efficacy of OP replacement therapy. Neurosphere grafts may be implanted directly into the central nervous system along with the modulating agent(s) using standard techniques.

Alternatively, a modulating agent may be administered alone or within a pharmaceutical composition. The duration and frequency of administration will be determined by such factors as the condition of the patient, and the type and severity of the patient's disease. Within particularly preferred embodiments of the invention, the compound of formula (I) or pharmaceutical composition may be administered at a dosage ranging from 0.1 mg/kg to 20 mg/kg, although more specific and preferred dosages may be determined using routine methodologies. Methods of administration include, for example, injection, intravenous or intrathecal (i.e., directly in cerebrospinal fluid).

Effective treatment of multiple sclerosis may be evidenced by any of the following criteria: EDSS (extended disability status scale), appearance of exacerbations or MRI (magnetic resonance imaging). The EDSS is a means to grade clinical impairment due to MS (Kurtzke, Neurology 33:1444, 1983), and a decrease of one full step defines an effective treatment in the context of the present invention (Kurtzke, Ann. Neurol. 36:573-79, 1994). Exacerbations are defined as the appearance of a new symptom that is attributable to MS and accompanied by an appropriate new neurologic abnormality (Sipe et al., Neurology 34:1368, 1984). Therapy is deemed to be effective if there is a statistically significant difference in the rate or proportion of exacerbation-free patients between the treated group and the placebo group or a statistically significant difference in the time to first exacerbation or duration and severity in the treated group compared to control group. MRI can be used to measure active lesions using gadolinium-DTPA-enhanced imaging (McDonald et al. Ann. Neurol. 36:14, 1994) or the location and extent of lesions using T₂-weighted techniques. The presence, location and extent of MS lesions may be determined by radiologists using standard techniques. Improvement due to therapy is established when there is a statistically significant improvement in an individual patient compared to baseline or in a treated group versus a placebo group.

Efficacy of the modulating agent in the context of prevention may be judged based on clinical measurements such as the relapse rate and EDSS. Other criteria include a change in area and volume of T2 images on MRI, and the number and volume of lesions determined by gadolinium enhanced images.

Within a related aspect, the present invention provides methods for facilitating migration of an N-cadherin expressing cell on astrocytes, comprising contacting an N-cadherin expressing cell with (a) a cell adhesion modulating agent that inhibits cadherin-mediated cell adhesion, wherein the modulating agent comprises a compound of formula (I) as provided herein; and (b) one or more astrocytes; and thereby facilitating migration of the N-cadherin expressing cell on the astrocytes. Preferred N-cadherin expressing cells include Schwann cells, oligodendrocytes and oligodendrocyte progenitor cells.

Within another aspect, modulating agents as described herein may be used for modulating the immune system of a mammal in any of several ways. Cadherins are expressed on immature B and T cells (thymocytes and bone marrow pre-B cells), as well as on specific subsets of activated B and T lymphocytes and some hematological malignancies (see Lee et al., J. Immunol. 152:5653-5659, 1994; Munro et al., Cellular Immunol. 169:309-312, 1996; Tsutsui et al., J. Biochem. 120:1034-1039, 1996; Cepek et al., Proc. Natl. Acad. Sci. USA 93:6567-6571, 1996). Modulating agents may generally be used to modulate specific steps within cellular interactions during an immune response or during the dissemination of malignant lymphocytes.

For example, a modulating agent as described herein may be used to treat diseases associated with excessive generation of otherwise normal T cells. Without wishing to be bound by any particular theory, it is believed that the interaction of cadherins on maturing T cells and B cell subsets contributes to protection of these cells from programmed cell death. A modulating agent may decrease such interactions, leading to the induction of programmed cell death. Accordingly, modulating agents may be used to treat certain types of diabetes and rheumatoid arthritis, particularly in young children where the cadherin expression on thymic pre-T cells is greatest.

Modulating agents may also be administered to patients afflicted with certain skin disorders (such as cutaneous lymphomas), acute B cell leukemia and excessive immune reactions involving the humoral immune system and generation of immunoglobulins, such as allergic responses and antibody-mediated graft rejection. In addition, patients with circulating cadherin-positive malignant cells (e.g., during regimes where chemotherapy or radiation therapy is eliminating a major portion of the malignant cells in bone marrow and other lymphoid tissue) may benefit from treatment with a compound of formula (I). Such treatment may also benefit patients undergoing transplantation with peripheral blood stem cells.

Preferred modulating agents for use within such methods include those that disrupt E-cadherin and/or N-cadherin mediated cell adhesion, such as agents that comprise a compound of formula (I) as described above. Alternatively, a separate modulator of integrin-mediated cell adhesion may be administered in conjunction with the modulating agent(s), either within the same pharmaceutical composition or separately.

Within the above methods, the modulating agent(s) are preferably administered systemically (usually by injection) or topically. A compound of formula (I) may be linked to a targeting agent. As noted above, a modulating agent may further be linked to a targeting agent. For example, targeting to the bone marrow may be beneficial. A suitable dosage is sufficient to effect a statistically significant reduction in the population of B and/or T cells that express cadherin and/or an improvement in the clinical manifestation of the disease being treated. Typical dosages range as described above.

Within further aspects, the present invention provides methods and kits for preventing pregnancy in a mammal. In general, disruption of E-cadherin function prevents the adhesion of trophoblasts and their subsequent fusion to form syncitiotrophoblasts. In one embodiment, one or more modulating agents as described herein may be incorporated into any of a variety of well known contraceptive devices, such as sponges suitable for intravaginal insertion (see, e.g., U.S. Pat. No. 5,417,224) or capsules for subdermal implantation. Other modes of administration are possible, however, including transdermal administration, for modulating agents linked to an appropriate targeting agent. Preferred modulating agents for use within such methods comprise a compound of formula (I). Alternatively, a separate modulator of integrin-mediated cell adhesion may be administered in conjunction with the modulating agent(s), either within the same pharmaceutical composition or separately.

Suitable methods for incorporation into a contraceptive device depend upon the type of device and are well known in the art. Such devices facilitate administration of the compound(s) of formula (I) to the uterine region and may provide a sustained release of the compound(s) of formula (I). In general, compound(s) of formula (I) may be administered via a contraceptive device at a dosage ranging from 0.1 to 20 mg/kg, although appropriate dosages may be determined by monitoring hCG levels in the urine. hCG is produced by the placenta, and levels of this hormone rise in the urine of pregnant women. The urine hCG levels can be assessed by radio-immunoassay using well known techniques. Kits for preventing pregnancy generally comprise a contraceptive device impregnated with one or more compounds of formula (I).

Alternatively, a sustained release formulation of one or more compounds of formula (I) may be implanted, typically subdermally, in a mammal for the prevention of pregnancy. Such implantation may be performed using well known techniques. Preferably, the implanted formulation provides a dosage as described above, although the minimum effective dosage may be determined by those of ordinary skill in the art using, for example, an evaluation of hCG levels in the urine of women.

The present invention also provides methods for increasing vasopermeability in a mammal by administering one or more modulating agents or pharmaceutical compositions. Within blood vessels, endothelial cell adhesion (mediated by N-cadherin) results in decreased vascular permeability. Accordingly, modulating agents as described herein may be used to increase vascular permeability. Within certain embodiments, preferred modulating agents for use within such methods include compounds of formula (I) capable of decreasing both endothelial and tumor cell adhesion. Such modulating agents may be used to facilitate the penetration of anti-tumor therapeutic or diagnostic agents (e.g., monoclonal antibodies) through endothelial cell permeability barriers and tumor barriers. Preferred modulating agents for use within such methods comprise a single compound of formula (I). Alternatively, a separate modulator of occludin mediated cell adhesion may be administered in conjunction with one or modulating agents, either within the same pharmaceutical composition or separately.

Within certain embodiments, preferred modulating agents for use within such methods include compounds of formula (I) capable of decreasing both endothelial and tumor cell adhesion. Such modulating agents may be used to facilitate the penetration of anti-tumor therapeutic or diagnostic agents (e.g., monoclonal antibodies) through endothelial cell permeability barriers and tumor barriers. For example, a modulating agent may comprise a compounds of formula (I). Alternatively, separate modulating agents capable of disrupting N- and E-cadherin mediated adhesion may be administered concurrently.

Treatment with a modulating agent may be appropriate, for example, prior to or concurrent with administration of an anti-tumor therapeutic or diagnostic agent (e.g., a monoclonal antibody or other macromolecule), an antimicrobial agent or an anti-inflammatory agent, in order to increase the concentration of such agents in the vicinity of the target tumor, organism or inflammation without increasing the overall dose to the patient. Modulating agents for use within such methods may be linked to a targeting agent to further increase the local concentration of modulating agent, although systemic administration of a vasoactive agent even in the absence of a targeting agent increases the perfusion of certain tumors relative to other tissues. Suitable targeting agents include antibodies and other molecules that specifically bind to tumor cells or to components of structurally abnormal blood vessels. For example, a targeting agent may be an antibody that binds to a fibrin degradation product or a cell enzyme such as a peroxidase that is released by granulocytes or other cells in necrotic or inflamed tissues.

Administration via intravenous injection or transdermal administration is generally preferred. Effective dosages are generally sufficient to increase localization of a subsequently administered diagnostic or therapeutic agent to an extent that improves the clinical efficacy of therapy of accuracy of diagnosis to a statistically significant degree. Comparison may be made between treated and untreated tumor host animals to whom equivalent doses of the diagnostic or therapeutic agent are administered. In general, dosages range as described above.

Within a further aspect, modulating agents as described herein may be used for controlled inhibition of synaptic stability, resulting in increased synaptic plasticity. Within this aspect, administration of one or more modulating agents may be advantageous for repair processes within the brain, as well as learning and memory, in which neural plasticity is a key early event in the remodeling of synapses. Cell adhesion molecules, particularly N-cadherin and E-cadherin, can function to stabilize synapses, and loss of this function is thought to be the initial step in the remodeling of the synapse that is associated with learning and memory (Doherty et al., J. Neurobiology, 26:437-446, 1995; Martin and Kandel, Neuron, 17:567-570, 1996; Fannon and Colman, Neuron, 17:423-434, 1996). Inhibition of cadherin function by administration of one or more modulating agents that inhibit cadherin function may stimulate learning and memory.

Preferred modulating agents for use within such methods include those that disrupt E-cadherin and/or N-cadherin mediated cell adhesion, such as agents that comprise a compounds of formula (I). Alternatively, a separate modulator of integrin and/or N-CAM mediated cell adhesion may be administered in conjunction with the modulating agent(s), either within the same pharmaceutical composition or separately. For such aspects, administration may be via encapsulation into a delivery vehicle such as a liposome, using standard techniques, and injection into, for example, the carotid artery. Alternatively, a modulating agent may be linked to a disrupter of the blood-brain barrier. In general dosages range as described above.

Within further aspects, compounds of formula (I) may be used to facilitate cell identification and sorting in vitro or imaging in vivo, permitting the selection of cells expressing different cadherins (or different cadherin levels). Preferably, the compound(s) of formula (I) for use in such methods are linked to a detectable marker. Suitable markers are well known in the art and include radionuclides, luminescent groups, fluorescent groups, enzymes, dyes, constant immunoglobulin domains and biotin. Within one preferred embodiment, a compound of formula (I) linked to a fluorescent marker, such as fluorescein, is contacted with the cells, which are then analyzed by fluorescence activated cell sorting (FACS).

Cadherin Antagonists

In certain embodiments, one or more compounds of the invention is used in conjunction with at least one other compound or agent or treatment, such as one or more additional cadherin antagonists, one or more anticancer agents, etc. An additional cadherin antagonist, for example, may may include essentially any compound capable of modulating a cadherin protein, particularly compounds capable of inhibiting at least one cadherin-mediated function or process, such as cell adhesion. Illustrative examples of various known cadherin antagonists that may be used in combination with the compounds herein described below.

a. Cadherin Antagonists Comprising HAV CAR Sequences

Certain peptide-based cadherin antagonists have been extensively described and are useful in the context of the present invention, e.g., U.S. Pat. Nos. 6,031,072; 6,417,325; 6,465,427; 6,780,845; 6,203,788; and WO05/012348, the contents of which are incorporated herein by reference in their entireties. Such agents represent classical cadherin antagonists and generally comprise linear and/or cyclic peptides containing the classical cadherin cell adhesion recognition (CAR) sequence HAV (i.e., His-Ala-Val), or may also be analogues, peptidomimetics or derivatives thereof.

In one embodiment, particular cadherin antagonists comprise cyclic peptides, or salts thereof, that comprise (1) an intramolecular covalent bond between two non-adjacent residues and (2) at least one classical cadherin cell adhesion recognition (CAR) sequence HAV (His-Ala-Val). The intramolecular bond may be a backbone to backbone, side-chain to backbone or side-chain to side-chain bond (i.e., terminal functional groups of a linear peptide and/or side chain functional groups of a terminal or interior residue may be linked to achieve cyclization). Preferred intramolecular bonds include, but are not limited to, disulfide, amide and thioether bonds. In addition to the classical cadherin CAR sequence HAV, a modulating agent may comprise additional CAR sequences, which may or may not be cadherin CAR sequences, and/or antibodies or fragments thereof that specifically recognize a CAR sequence. Additional CAR sequences may be present within the cyclic peptide containing the HAV sequence, within a separate cyclic peptide component of the modulating agent and/or in a non-cyclic portion of the modulating agent.

Certain preferred HAV-containing cyclic peptides satisfy the formula:

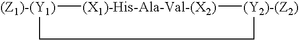

wherein X₁, and X₂ are optional, and if present, are independently selected from the group consisting of amino acid residues and combinations thereof in which the residues are linked by peptide bonds, and wherein X₁ and X₂ independently range in size from 0 to 10 residues, such that the sum of residues contained within X₁ and X₂ ranges from 1 to 12; wherein Y₁ and Y₂ are independently selected from the group consisting of amino acid residues, and wherein a covalent bond is formed between residues Y₁ and Y₂; and wherein Z₁ and Z₂ are optional, and if present, are independently selected from the group consisting of amino acid residues and combinations thereof in which the residues are linked by peptide bonds.

Within certain embodiments, a cyclic peptide may comprise an N-acetyl group (i.e., the amino group present on the amino terminal residue of the peptide prior to cyclization is acetylated) or an N-formyl group (i.e., the amino group present on the amino terminal residue of the peptide prior to cyclization is formylated), or the amino group present on the amino terminal residue of the peptide prior to cyclization is mesylated. One preferred cyclic peptide, for example, is N-Ac-CHAVC-NH₂ (SEQ ID NO: 1). Another preferred cyclic peptide is N-Ac-CHAVC-Y-NH₂ (SEQ ID NO:2). Other cyclic peptides include, but are not limited to: N-Ac-CHAVDC-NH₂ (SEQ ID NO:3), N-Ac-CHAVDIC-NH₂ (SEQ ID NO:4), N-Ac-CHAVDINC-NH₂ (SEQ ID NO:5), N-Ac-CHAVDINGC-NH₂ (SEQ ID NO:6), N-Ac-CAHAVC-NH₂ (SEQ ID NO:7), N-Ac-CAHAVDC-NH₂ (SEQ ID NO:8), N-Ac-CAHAVDIC-NH₂ (SEQ ID NO:9), N-Ac-CRAHAVDC-NH₂ (SEQ ID NO:10), N-Ac-CLRAHAVC-NH₂ (SEQ ID NO:11), N-Ac-CLRAHAVDC-NH₂ (SEQ ID NO: 12), N-Ac-CSHAVC-NH₂ (SEQ ID NO: 13), N-Ac-CFSHAVC-NH₂ (SEQ ID NO: 14), N-Ac-CLFSHAVC-NH₂ (SEQ ID NO: 15), N-Ac-CHAVSC-NH₂ (SEQ ID NO: 16), N-Ac-CSHAVSC-NH₂ (SEQ ID NO:17), N-Ac-CSHAVSSC-NH₂ (SEQ ID NO:18), N-Ac-CHAVSSC-NH₂ (SEQ ID NO:19), N-Ac-KHAVD-NH₂ (SEQ ID NO:20), N-Ac-DHAVK-NH₂ (SEQ ID NO:21), N-Ac-KHAVE-NH₂ (SEQ ID NO:22), N-Ac-AHAVDI-NH₂ (SEQ ID NO:23), N-Ac-SHAVDSS-NH₂ (SEQ ID NO:24), N-Ac-KSHAVSSD-NH₂ (SEQ ID NO:25), N-Ac-CHAVC-S-NH₂ (SEQ ID NO:26), N-Ac-S-CHAVC-NH₂ (SEQ ID NO:27), N-Ac-CHAVC-SS-NH₂ (SEQ ID NO:28), N-Ac-S-CHAVC-S-NH₂ (SEQ ID NO:29), N-Ac-CHAVC-T-NH₂ (SEQ ID NO:30), N-Ac-CHAVC-E-NH₂ (SEQ ID NO:31), N-Ac-CHAVC-D-NH₂ (SEQ ID NO:32), N-Ac-CHAVYC-NH₂ (SEQ ID NO:33), CH₃—SO₂—HN-CHAVC-Y-NH₂ (SEQ ID NO:34), CH₃—SO₂—HN-CHAVC-NH₂ (SEQ ID NO:35), HC(O)—NH-CHAVC-NH₂ (SEQ ID NO:36), N-Ac-CHAVPen-NH₂ (SEQ ID NO:37), N-Ac-PenHAVC-NH₂ (SEQ ID NO:38) and N-Ac-CHAVPC-NH₂ (SEQ ID NO:39).

In addition to CAR sequence(s), cyclic peptides generally comprise at least one additional residue, such that the size of the cyclic peptide ring ranges from 4 to about 15 residues, preferably from 5 to 10 residues. Such additional residue(s) may be present on the N-terminal and/or C-terminal side of a CAR sequence, and may be derived from sequences that flank the HAV sequence within one or more naturally occurring cadherins (e.g., N-cadherin, E-cadherin, P-cadherin, R-cadherin or other cadherins containing the HAV sequence) with or without amino acid substitutions and/or other modifications. Database accession numbers for representative naturally occurring cadherins are as follows: human N-cadherin M34064, mouse N-cadherin M31131 and M22556, cow N-cadherin X53615, human P-cadherin X63629, mouse P-cadherin X06340, human E-cadherin Z13009, mouse E-cadherin X06115. Alternatively, additional residues present on one or both sides of the CAR sequence(s) may be unrelated to an endogenous sequence (e.g., residues that facilitate cyclization).

Within certain embodiments, relatively small cyclic peptides that do not contain significant sequences flanking the HAV sequence are used for modulating N-cadherin and E-cadherin mediated cell adhesion.

b. Cadherin Antagonists Comprising Trp-Containing CAR Sequences

Additional cadherin antagonists useful in combinations of the present invention include agents comprising Trp-containing CAR sequences that modulate classical cadherins, as well as peptidomimetics, analogues and derivatives thereof, such as those described in U.S. patent application Ser. No. 10/714,556; US Patent Publication No. 2005/0129676, and PCT Publication No. WO04/044000, the contents of which are incorporated herein by reference in their entireties.

For example, illustrative Trp-containing CAR sequences may comprise the consensus sequence: Asp/Glu-Trp-Val-Ile/Val/Met-Pro/Ala-Pro (SEQ ID NO:40), wherein “Asp/Glu” is an amino acid that is either Asp or Glu, “Ile/Val/Met” is an amino acid that is Ile, Val or Met, and “Pro/Ala” is either Pro or Ala. Particular Trp-containing CAR sequences or conservative analogues thereof include, but are not limited to, DWV, DWVI (SEQ ID NO:41), DWVV (SEQ ID NO: 42), DWVM (SEQ ID NO:43), DWVIP (SEQ ID NO:44), DWVIA (SEQ ID NO:45), DWVVP (SEQ ID NO:46), DWVVPP (SEQ ID NO:47), DWVVAP (SEQ ID NO:48), DWVMPP (SEQ ID NO:49), DWVMAP (SEQ ID NO:50), EWV, EWVI (SEQ ID NO:51), EWVV (SEQ ID NO:52), EWVM (SEQ ID NO:53), EWVIP (SEQ ID NO:54), EWVIA (SEQ ID NO:55), EWVVP (SEQ ID NO:56), EWVVPP (SEQ ID NO:57), EWVVAP (SEQ ID NO:58), EWVMPP (SEQ ID NO:59), EWVMAP (SEQ ID NO:60), WVI, WVIP (SEQ ID NO:61), WVIA (SEQ ID NO:62), WVV, WVVP (SEQ ID NO:63), WVVA (SEQ ID NO:64), WVM, WVMP (SEQ ID NO:65), WVMA (SEQ ID NO:66), WVIPP (SEQ ID NO:67), WVIAP (SEQ ID NO:68), WVVPP (SEQ ID NO:69), WVVAP (SEQ ID NO:70), WVMPP (SEQ ID NO:71), WVMAP (SEQ ID NO:72), DWI, DWII (SEQ ID NO:73), DWIV (SEQ ID NO:74), DWIM (SEQ ID NO:75), DWIIP (SEQ ID NO:76), DWIIA (SEQ ID NO:77), DWIVP (SEQ ID NO:78), DWIVPP (SEQ ID NO:79), DWIVAP (SEQ ID NO:80), DWIMPP (SEQ ID NO:81), DWIMAP (SEQ ID NO:82), EWI, EWII (SEQ ID NO:83), EWIV (SEQ ID NO:84), EWIM (SEQ ID NO:85), EWIIP (SEQ ID NO:86), EWIIA (SEQ ID NO:87), EWIVP (SEQ ID NO:88), EWIVPP (SEQ ID NO:89), EWIVAP (SEQ ID NO:90), EWIMPP (SEQ ID NO:91), EWIMAP (SEQ ID NO:92), WII, WIIP (SEQ ID NO:93), WIIA (SEQ ID NO:94), WIV, WIVP (SEQ ID NO:95), WIVA (SEQ ID NO:96), WIM, WIMP (SEQ ID NO:97), WIMA (SEQ ID NO:98), WIIPP (SEQ ID NO:99), WIIAP (SEQ ID NO:100), WIVPP (SEQ ID NO:101), WIVAP (SEQ ID NO:102), WIMPP (SEQ ID NO:103), WIMAP (SEQ ID NO:104), DWL, DWLI (SEQ ID NO:105), DWLV (SEQ ID NO:106), DWLM (SEQ ID NO:107), DWLIP (SEQ ID NO:108), DWLIA (SEQ ID NO:109), DWLVP (SEQ ID NO:10), DWLVPP (SEQ ID NO:111), DWLVAP (SEQ ID NO:112), DWLMPP (SEQ ID NO:113), DWLMAP (SEQ ID NO:114), EWL, EWLI (SEQ ID NO:115), EWLV (SEQ ID NO:116), EWLM (SEQ ID NO:117), EWLIP (SEQ ID NO:118), EWLIA (SEQ ID NO:119), EWLVP (SEQ ID NO:120), EWLVPP (SEQ ID NO:121), EWLVAP (SEQ ID NO:122), EWLMPP (SEQ ID NO:123), EWLMAP (SEQ ID NO:124), WLI, WLIP (SEQ ID NO:125), WLIA (SEQ ID NO:126), WLV, WLVP (SEQ ID NO:127), WLVA (SEQ ID NO:128), WLM, WLMP (SEQ ID NO:129), WLMA (SEQ ID NO:130), WLIPP (SEQ ID NO:131), WLIAP (SEQ ID NO:132), WLVPP (SEQ ID NO:133), WLVAP (SEQ ID NO:134), WLMPP (SEQ ID NO:135), WLMAP (SEQ ID NO:136), DWVL (SEQ ID NO:137), DWIL (SEQ ID NO:138), DWLL (SEQ ID NO:139), EWVL (SEQ ID NO:140), EWIL (SEQ ID NO:141), EWLL (SEQ ID NO:142), DWVLP (SEQ ID NO:143), DWILP (SEQ ID NO:144), DWLLP (SEQ ID NO:145), EWVLP (SEQ ID NO:146), EWILP (SEQ ID NO:147), EWLLP (SEQ ID NO:148), DWVLA (SEQ ID NO:149), DWILA (SEQ ID NO:150), DWLLA (SEQ ID NO:151), EWVLA (SEQ ID NO:152), EWILA (SEQ ID NO:153), EWLLA (SEQ ID NO:154), DWVLPP (SEQ ID NO:155), DWILPP (SEQ ID NO:156), DWLLPP (SEQ ID NO:157), EWVLPP (SEQ ID NO:158), EWILPP (SEQ ID NO:159), EWLLPP (SEQ ID NO:160), DWVLAP (SEQ ID NO:161), DWILAP (SEQ ID NO:162), DWLLAP (SEQ ID NO:163), EWVLAP (SEQ ID NO:164), EWILAP (SEQ ID NO:165), EWLLAP (SEQ ID NO:166), WVL, WIL, WLL, WVLP (SEQ ID NO:167), WILP (SEQ ID NO:168), WLLP (SEQ ID NO:169), WVLA (SEQ ID NO:170), WILA (SEQ ID NO:171), WLLA (SEQ ID NO:172), WVLPP (SEQ ID NO:173), WILPP (SEQ ID NO:174), WLLPP (SEQ ID NO:175), WVLAP (SEQ ID NO:176), WILAP (SEQ ID NO:177), and WLLAP (SEQ ID NO:178).

Trp-containing CAR sequences can also be present in cyclic peptide structures, illustrative examples of which may have the following structures:

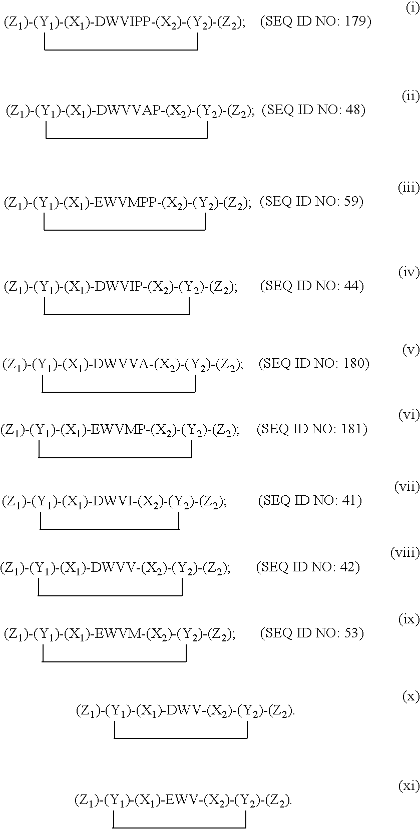

In these structures, X₁ and X₂ are optional, and if present, are amino acid residues or combinations of amino acid residues linked by peptide bonds. X₁ and X₂ may be identical to, or different from, each other. In general, X₁ and X₂ independently range in size from 0 to 10 residues, such that the sum of residues contained within X₁ and X₂ ranges from 1 to 12. Y₁ and Y₂ are amino acid residues, and a covalent bond is formed between residues Y₁ and Y₂. Y₁ and Y₂ may be identical to, or different from, each other. Z₁ and Z₂ are optional, and if present, are amino acid residues or combinations of amino acid residues linked by peptide bonds. Z₁ and Z₂ may be identical to, or different from, each other.

Other cadherin antagonists useful in the present invention include agents comprising Trp-containing CAR sequences that modulate non-classical and atypical cadherins, as well as peptidomimetics, analogues and derivatives thereof, such as those described in US Patent Publication No. 2004/0175361, the content of which is incorporated herein by reference in its entirety.

For example, certain atypical cadherin Trp-containing CAR sequences share the consensus sequence:

(SEQ ID NO: 268)

Gly/Asp/Ser-Trp-Val/Ile/Met-Trp-Asn-Gln

Within the consensus sequence, “Gly/Asp/Ser” indicates an amino acid that is Gly, Asp or Ser; and “Val/Ile/Met” indicates an amino acid that is Val, Ile or Met. Representative atypical cadherin Trp-containing CAR sequences are provided within Table I. Trp-containing CAR sequences specifically provided herein further include portions of such representative Trp-containing CAR sequences, as well as polypeptides that comprise at least a portion of such sequences. Additional atypical cadherin Trp-containing CAR sequences may be identified based on sequence homology to the atypical cadherin Trp-containing CAR sequences provided herein, and based on the ability of a peptide comprising such a sequence to modulate an atypical cadherin-mediated function within a representative assay described herein. Within certain embodiments, an antagonist comprises at least three, four, five and six consecutive residues of an atypical cadherin Trp-containing CAR sequence that satisfies the above consensus sequence.

Exemplary Trp-containing CAR sequences for atypical cadherins include, but are not limited to GWV, GWVW (SEQ ID NO:269), GWVWN (SEQ ID NO:270), GWVWNQ (SEQ ID NO:271), WVW, WVWN (SEQ ID NO:272), WVWNQ (SEQ ID NO:273), DWI, DWIW (SEQ ID NO:274), DWIWN (SEQ ID NO:275), DWIWNQ (SEQ ID NO:276), WIW, WIWN (SEQ ID NO:277), WIWNQ (SEQ ID NO:278), SWM, SWMW (SEQ ID NO:279), SWMWN (SEQ ID NO:280), SWMWNQ (SEQ ID NO:281), WMW, WMWN (SEQ ID NO:282), WMWNQ (SEQ ID NO:283), SWV, SWVW (SEQ ID NO:284), SWVWN (SEQ ID NO:285), SWVWNQ (SEQ ID NO:286), GWM, GWMW (SEQ ID NO:287), GWMWN (SEQ ID NO:288), GWMWNQ (SEQ ID NO:289), AWV, AWVI (SEQ ID NO:290), AWVIP (SEQ ID NO:291), AWVIPP (SEQ ID NO:292), WVI, WVIP (SEQ ID NO:293), WVIPP (SEQ ID NO:294), GWVWNQF (SEQ ID NO:295), GWVWNQFF (SEQ ID NO:296), GWVWNQFFV (SEQ ID NO:297), WVWNQF (SEQ ID NO:298), WVWNQFF (SEQ ID NO:299), WVWNQFFV (SEQ ID NO:300), RGW, RGWV (SEQ ID NO:301), RGWVW (SEQ ID NO:302), RGWVWN (SEQ ID NO:303), RGWVWNQ (SEQ ID NO:304), RGWVWNQF (SEQ ID NO:305), RGWVWNQFF (SEQ ID NO:306), RGWVWNQFFV (SEQ ID NO:307), KRGW (SEQ ID NO:308), KRGWV (SEQ ID NO:309), KRGWVW (SEQ ID NO:310), KRGWVWN (SEQ ID NO:311), KRGWVWNQ (SEQ ID NO:312), KRGWVWNQF (SEQ ID NO:313), KRGWVWNQFF (SEQ ID NO:314), KRGWVWNQFFV (SEQ ID NO:315), DWIWNQM (SEQ ID NO:316), DWIWNQMH (SEQ ID NO:317), DWIWNQMHI (SEQ ID NO:318), WIWNQM (SEQ ID NO:319), WIWNQMH (SEQ ID NO:320), WIWNQMHI (SEQ ID NO:321), RDW, RDWI (SEQ ID NO:322), RDWIW (SEQ ID NO:323), RDWIWN (SEQ ID NO:324), RDWIWNQ (SEQ ID NO:325), RDWIWNQM (SEQ ID NO:326), RDWIWNQMH (SEQ ID NO:327), RDWIWNQMHI (SEQ ID NO:328), KRDW (SEQ ID NO:329), KRDWI (SEQ ID NO:330), KRDWIW (SEQ ID NO:331), KRDWIWN (SEQ ID NO:332), KRDWIWNQ (SEQ ID NO:333), KRDWIWNQM (SEQ ID NO:334), KRDWIWNQMH (SEQ ID NO:335), KRDWIWNQMHI (SEQ ID NO:336), SWMWNQF (SEQ ID NO:337), SWMWNQFF (SEQ ID NO:338), SWMWNQFFL (SEQ ID NO:339), WMWNQF (SEQ ID NO:340), WMWNQFF (SEQ ID NO:341), WMWNQFFL (SEQ ID NO:342), RSW, RSWM (SEQ ID NO:343), RSWMW (SEQ ID NO:344), RSWMWN (SEQ ID NO:345), RSWMWNQ (SEQ ID NO:346), RSWMWNQF (SEQ ID NO:347), RSWMWNQFF (SEQ ID NO:348), RSWMWNQFFL (SEQ ID NO:349), KRSW (SEQ ID NO:350), KRSWM (SEQ ID NO:351), KRSWMW (SEQ ID NO:352), KRSWMWN (SEQ ID NO:353), KRSWMWNQ (SEQ ID NO:354), KRSWMWNQF (SEQ ID NO:355), KRSWMWNQFF (SEQ ID NO:356), KRSWMWNQFFL (SEQ ID NO:357), SWVWNQF (SEQ ID NO:358), SWVWNQFF (SEQ ID NO:359), SWVWNQFFV (SEQ ID NO:360), WVWNQF (SEQ ID NO:361), WVWNQFF (SEQ ID NO:362), WVWNQFFV (SEQ ID NO:363), RSWV (SEQ ID NO:364), RSWVW (SEQ ID NO:365), RSWVWN (SEQ ID NO:366), RSWVWNQ (SEQ ID NO:367), RSWVWNQF (SEQ ID NO:368), RSWVWNQFF (SEQ ID NO:369), RSWVWNQFFV (SEQ ID NO:370), KRSWV (SEQ ID NO:371), KRSWVW (SEQ ID NO:372), KRSWVWN (SEQ ID NO:373), KRSWVWNQ (SEQ ID NO:374), KRSWVWNQF (SEQ ID NO:375), KRSWVWNQFF (SEQ ID NO:376), KRSWVWNQFFV (SEQ ID NO:377), GWVWNQM (SEQ ID NO:378), GWVWNQMF (SEQ ID NO:379), GWVWNQMFV (SEQ ID NO:380), RGWVWNQM (SEQ ID NO:381), RGWVWNQMF (SEQ ID NO:382), RGWVWNQMFV (SEQ ID NO:383), KRGWVWNQM (SEQ ID NO:384), KRGWVWNQMFV (SEQ ID NO:385), GWVWNQFFL (SEQ ID NO:386), RGWVWNQFFL (SEQ ID NO:387), KRGWVWNQFFL (SEQ ID NO:388), AWVIPPI (SEQ ID NO:389), AWVIPPIS (SEQ ID NO:390), AWVIPPISV (SEQ ID NO:391), WVIPPI (SEQ ID NO:392), WVIPPIS (SEQ ID NO:393), WVIPPISV (SEQ ID NO:394), RAW, RAWV (SEQ ID NO:395), RAWVI (SEQ ID NO:396), RAWVIP (SEQ ID NO:397), RAWVIPP (SEQ ID NO:398), RAWVIPPI (SEQ ID NO:399), RAWVIPPIS (SEQ ID NO:400), RAWVIPPISV (SEQ ID NO:401), KRAW (SEQ ID NO:402), KRAWV (SEQ ID NO:403), KRAWVI (SEQ ID NO:404), KRAWVIP (SEQ ID NO:405), KRAWVIPP (SEQ ID NO:406), KRAWVIPPI (SEQ ID NO:407), KRAWVIPPIS (SEQ ID NO:408), VWN, VWNQ (SEQ ID NO:409), VWNQM (SEQ ID NO:410), VWNQF (SEQ ID NO:411), VWNQMF (SEQ ID NO:412), VWNQFF (SEQ ID NO:413), WNQ, WNQM (SEQ ID NO:414), WNQF (SEQ ID NO:415), WNQFF (SEQ ID NO:416), IWN, IWNQ (SEQ ID NO:417), IWNQM (SEQ ID NO:418), IWNQMH (SEQ ID NO:419), WNQM (SEQ ID NO:420), WNQMH (SEQ ID NO:421), MWN, MWNQ (SEQ ID NO:422), MWNQF (SEQ ID NO:423), and MWNQFF (SEQ ID NO:424).

Other atypical cadherin antagonists are present within a cyclic peptide ring comprising the sequence G/S/D-W-V/M/I-W-N-Q (SEQ ID NO:268), the sequence AWVIPP (SEQ ID NO:292), or a portion thereof. Exemplary cyclic peptides have the following formula:

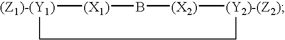

In this formula, B represents an amino acid sequence selected from the following sequences: DWIWNQ (SEQ ID NO:276), SWMWNQ (SEQ ID NO:281), SWVWNQ (SEQ ID NO:286), GWVWNQ (SEQ ID NO:271), AWVIPP (SEQ ID NO:292), GWVWN (SEQ ID NO:270), DWIWN (SEQ ID NO:275), SWMWN (SEQ ID NO:280), SWVWN (SEQ ID NO:285), GWVWN (SEQ ID NO:270), AWVIP (SEQ ID NO:291), GWVW (SEQ ID NO:269), DWIW (SEQ ID NO:274), SWMW (SEQ ID NO:279), SWVW (SEQ ID NO:284), GWVW (SEQ ID NO:269), AWVI (SEQ ID NO:290), GWV, DWI, SWM, SWV, GWV, AWV, VWN, VWNQ (SEQ ID NO:409), VWNQM (SEQ ID NO:410), VWNQF (SEQ ID NO:411), VWNQMF (SEQ ID NO:412), VWNQFF (SEQ ID NO:413), WNQ, WNQM (SEQ ID NO:414), WNQF (SEQ ID NO:415), WNQFF (SEQ ID NO:416), IWN, IWNQ (SEQ ID NO:417), IWNQM (SEQ ID NO:418), IWNQMH (SEQ ID NO:419), WNQM (SEQ ID NO:420), WNQMH (SEQ ID NO:421), MWN, MWNQ (SEQ ID NO:422), MWNQF (SEQ ID NO:423), and MWNQFF (SEQ ID NO:424). X₁ and X₂ are optional, and if present, are amino acid residues or combinations of amino acid residues linked by peptide bonds. X₁ and X₂ may be identical to, or different from, each other. In general, X₁ and X₂ independently range in size from 0 to 10 residues, such that the sum of residues contained within X₁ and X₂ ranges from 1 to 12. Y₁ and Y₂ are amino acid residues, and a covalent bond is formed between residues Y₁ and Y₂. Y₁ and Y₂ may be identical to, or different from, each other. Z₁ and Z₂ are optional, and if present, are amino acid residues or combinations of amino acid residues linked by peptide bonds. Z₁ and Z₂ may be identical to, or different from, each other.

c. Cadherin Antagonists Comprising HAV-BM CAR Sequences

Other cadherin antagonists for use in combinations of the invention comprise compounds referred to as HAV-binding motif (HAV-BM) sequences, such as those described, e.g., in U.S. Pat. Nos. 6,277,824; 6,472,368; and 6,806,255. Such agents generally comprise an HAV-BM sequence, or an analogue, peptidomimetic or derivative thereof. In a particular embodiment, the HAV-BM sequence comprises the sequence: (a) Ile/Val-Phe-Aaa-Ile-Baa-Caa-Daa-Ser/Thr-Gly-Eaa-Leu/Met (SEQ ID NO:182), wherein Aaa, Baa, Caa, Daa and Eaa are independently selected from the group consisting of amino acid residues; or comprises the sequence Trp-Leu-Aaa-Ile-Asp/Asn-Baa-Caa-Daa-Gly-Gln-Ile (SEQ ID NO:183), wherein Aaa, Baa, Caa and Daa are independently selected from the group consisting of amino acid residues.

Certain illustrative HAV-BM sequences include, but are not limited to, sequences selected from the group consisting of: IFIINPISGQL (SEQ ID NO: 184), IFILNPISGQL (SEQ ID NO:185), VFAVEKETGWL (SEQ ID NO: 186), VFSINSMSGRM (SEQ ID NO:187), VFIIERETGWL (SEQ ID NO:188), VFTIEKESGWL (SEQ ID NO: 189), VFNIDSMSGRM (SEQ ID NO: 190), WLKIDSVNGQI (SEQ ID NO: 191), WLKIDPVNGQI (SEQ ID NO: 192), WLAMDPDSGQV (SEQ ID NO:193), WLHINATNGQI (SEQ ID NO: 194), WLEINPDTGAI (SEQ ID NO: 195), WLAVDPDSGQI (SEQ ID NO: 196), WLEINPETGAI (SEQ ID NO: 197), WLHINTSNGQI (SEQ ID NO: 198), NLKIDPVNGQI (SEQ ID NO:199), LKIDPVNGQI (SEQ ID NO:200) and analogues of the foregoing sequences that retain at least seven consecutive residues (e.g., INPISGQ (SEQ ID NO:201), LNPISGQ (SEQ ID NO:202), IDPVSGQ (SEQ ID NO:203) or KIDPVNGQ (SEQ ID NO:204)), wherein the ability of the analogue to modulate a cadherin-mediated process is not diminished. Alternatively, an agent may be an HAV-BM sequence that comprises at least five consecutive residues of a peptide selected from the group consisting of INPISGQ (SEQ ID NO:201), LNPISGQ (SEQ ID NO:202), NLKIDPVNGQI (SEQ ID NO:203) and WLKIDPVNGQI (SEQ ID NO:204). For example, the agent may comprise a sequence selected from the group consisting of PISGQ (SEQ ID NO:205), PVNGQ (SEQ ID NO:206), PVSGR (SEQ ID NO:207), IDPVN (SEQ ID NO:208), INPIS (SEQ ID NO:209) and KIDPV (SEQ ID NO:210).

An HAV-BM sequence may be present within a linear peptide or a cyclic peptide. Certain illustrative cyclic peptides include, but are not limited to, the following structures:

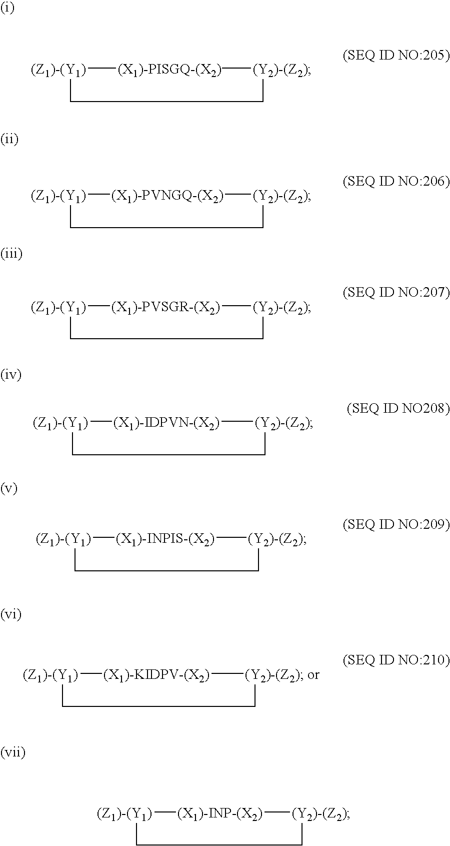

wherein X₁, and X₂ are optional, and if present, are independently selected from the group consisting of amino acid residues and combinations thereof in which the residues are linked by peptide bonds, and wherein X₁ and X₂ independently range in size from 0 to 10 residues, such that the sum of residues contained within X₁ and X₂ ranges from 1 to 12; wherein Y₁ and Y₂ are independently selected from the group consisting of amino acid residues, and wherein a covalent bond is formed between residues Y₁ and Y₂; and wherein Z₁ and Z₂ are optional, and if present, are independently selected from the group consisting of amino acid residues and combinations thereof in which the residues are linked by peptide bonds. Such cyclic peptides may contain modifications. For example, Y₁ may comprise an N-acetyl group and/or Y₂ may comprise a C-terminal amide group. Cyclization may be achieved in any of a variety of ways, such as covalent linkage of Y₁ and Y₂ via a disulfide, amide or thioether bond.

In addition to the illustrative peptide-based CAR sequences and structures discussed herein, suitable cadherin antagonists for use in the invention may also comprise analogues, peptidomimetics and derivatives thereof, as discussed herein and in the references incorporated herein.

d. Antibody-Based Cadherin Antagonists

Other illustrative cadherin antagonists used in the combinations of the invention may comprise antibodies, or antigen-binding fragments thereof, that are capable of modulating one or more cadherin-mediated processes or functions. For example, antibodies, and antigen-binding fragments thereof, may include those that specifically bind to a region of a cadherin and as a result antagonize one or more functions or processes mediated by the cadherin, such as cell adhesion. Particular antibodies, and antigen-binding fragments thereof, effective as cadherin antagonists, include antibodies capable of binding one or more CAR sequences described above and/or described in one or more of the references incorporated by reference herein (e.g., U.S. Pat. Nos. 6,031,072; 6,417,325; 6,465,427; 6,780,845; 6,203,788; WO05/012348; U.S. patent application Ser. No. 10/714,556; US Patent Publication Nos. 2005/0129676, 2005/0215482, 2005/0222037, 2005/0203025, 2004/0175361, PCT Publication No. WO04/044000; U.S. Pat. Nos. 6,277,824; 6,472,368; and 6,806,255).

An antibody, or antigen-binding fragment thereof, is said to “specifically bind” to a cadherin sequence (with or without flanking amino acids) if it reacts at a detectable level (within, for example, an ELISA, as described by Newton et al., Develop. Dynamics 197:1-13, 1993) with a peptide containing that sequence, and does not react at a detectable level, within the same or similar assay, with peptides containing a different sequence or a sequence in which the order of amino acid residues in the sequence and/or flanking sequence is different or has been altered.

Antibodies and fragments thereof may be prepared using standard techniques. See, e.g., Harlow and Lane, Antibodies: A Laboratory Manual, Cold Spring Harbor Laboratory, 1988. In one such technique, an immunogen comprising a CAR sequence is initially injected into any of a wide variety of mammals (e.g., mice, rats, rabbits, sheep or goats). Small immunogens (i.e., less than about 20 amino acids) should be joined to a carrier protein, such as bovine serum albumin or keyhole limpet hemocyanin. Following one or more injections, the animals are bled periodically. Polyclonal antibodies specific for the CAR sequence may then be purified from such antisera by, for example, affinity chromatography using the modulating agent or antigenic portion thereof coupled to a suitable solid support.

Monoclonal antibodies specific for a cadherin sequence may be prepared, for example, using the technique of Kohler and Milstein, Eur. J. Immunol. 6:511-519, 1976, and improvements thereto. Briefly, these methods involve the preparation of immortal cell lines capable of producing antibodies having the desired specificity from spleen cells obtained from an animal immunized as described above. The spleen cells are immortalized by, for example, fusion with a myeloma cell fusion partner, preferably one that is syngeneic with the immunized animal. Single colonies are selected and their culture supernatants tested for binding activity against the modulating agent or antigenic portion thereof. Hybridomas having high reactivity and specificity are preferred.

Monoclonal antibodies may be isolated from the supernatants of growing hybridoma colonies, with or without the use of various techniques known in the art to enhance the yield. Contaminants may be removed from the antibodies by conventional techniques, such as chromatography, gel filtration, precipitation, and extraction. Antibodies having the desired activity may generally be identified using immunofluorescence analyses of tissue sections, cell or other samples where the target cadherin is localized.

Within certain embodiments, antigen-binding fragments of antibodies are employed. Such fragments include Fab fragments, which may be prepared using standard techniques. Briefly, immunoglobulins may be purified from rabbit serum by affinity chromatography on Protein A bead columns (Harlow and Lane, Antibodies: A Laboratory Manual, Cold Spring Harbor Laboratory, 1988; see especially page 309) and digested by papain to yield Fab and Fc fragments. The Fab and Fc fragments may be separated by affinity chromatography on protein A bead columns (Harlow and Lane, 1988, pages 628-29).

e. Peptidomimetic & Small Molecule-Based N-Cadherin Antagonists

Still further cadherin antagonists useful in the combinations of the invention include peptidomimetics and small molecules having a three-dimensional structure that is substantially similar to a three-dimensional structure of a cyclic peptide antagonist that comprises the CAR sequence HAV within a cyclic peptide ring, such as those described in U.S. patent application Ser. No. 10/412,701 and PCT Publication No. WO01/53331, the contents of which are incorporated herein by reference in their entireties.

f. Other Cadherin Antagonists

Other cadherin antagonists useful in the combinations of the present invention include, for example, those capable of modulating non-classical cadherins, such as OB-cadherin and VE-cadherin. Illustrative non-classical cadherin antagonists include, for example, those described in US Patent Publication Nos. 2005/0215482; 2005/0222037; and 2005/0203025, the contents of which are incorporated herein by reference in their entireties.

Illustrative examples of non-classical cadherin CAR sequence have the formula:

(SEQ ID NO: 211)

Aaa-Phe-Baa-Ile/Leu/Val-Asp/Asn/Glu-Caa-Daa-Ser/
Thr/Asn-Gly

wherein Aaa, Baa, Caa and Daa are independently selected amino acid residues; Ile/Leu/Val is an amino acid that is selected from the group consisting of isoleucine, leucine and valine, Asp/Asn/Glu is an amino acid that is selected from the group consisting of aspartate, asparagine and glutamate; and Ser/Thr/Asn is an amino acid that is selected from the group consisting of serine, threonine or asparagine. For other antagonists as described, the non-classical cadherin CAR sequence consists of at least three consecutive amino acid residues, and preferably at least five consecutive amino acid residues, of a non-classical cadherin, wherein the consecutive amino acids are present within a region of the non-classical cadherin having the formula recited above. Other agents may comprise at least nine consecutive amino acid residues of a non-classical cadherin, wherein the nine consecutive amino acid residues comprise a region having a formula as recited above.

Within certain specific embodiments, an antagonist is a peptide ranging in size from 3 to 50, preferably from 4 to 16, amino acid residues.

Within other embodiments, an antagonist comprises a non-classical cadherin CAR sequence that is present within a cyclic peptide. Such cyclic peptides may have the formula:

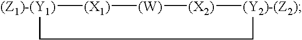

wherein W is a tripeptide selected from the group consisting of EEY, DDK, EAQ, DAE, NEN, ESE, DSG, DEN, EPK, DAN, EEF, NDV, DET, DPK, DDT, DAN, DKF, DEL, DAD, NNK, DLV, NRD, DPS, NQK, NRN, NKD, EKD, ERD, DPV, DSV, DLY, DSN, DSS, DEK, NEK; RAL, YAL, YAT, FAT and YAS wherein X₁, and X₂ are optional, and if present, are independently selected from the group consisting of amino acid residues and combinations thereof in which the residues are linked by peptide bonds, and wherein X₁ and X₂ independently range in size from 0 to 10 residues, such that the sum of residues contained within X₁ and X₂ ranges from 1 to 12; wherein Y₁ and Y₂ are independently selected from the group consisting of amino acid residues, and wherein a covalent bond is formed between residues Y₁ and Y₂; and wherein Z₁ and Z₂ are optional, and if present, are independently selected from the group consisting of amino acid residues and combinations thereof in which the residues are linked by peptide bonds.

The present invention also employs antagonists that comprise an antibody or antigen-binding fragment thereof that specifically binds to a non-classical cadherin CAR sequence and modulates a non-classical cadherin-mediated function,

Within further aspects, the present invention employs antagonists comprising a non-peptide mimetic of any one of the non-classical cadherin CAR sequences provided above and/or in the references incorporated herein.

Certain illustrative OB-cadherin antagonists comprise: (a) one or more OB-cadherin CAR sequences selected from the group consisting of DDK, IDDK (SEQ ID NO:212) DDKS (SEQ ID NO:213), VIDDK (SEQ ID NO:214), IDDKS (SEQ ID NO:215), VIDDKS (SEQ ID NO:216), DDKSG (SEQ ID NO:217), IDDKSG (SEQ ID NO:218), VIDDKSG (SEQ ID NO:219), FVIDDK (SEQ ID NO:220), FVIDDKS (SEQ ID NO:221), FVIDDKSG (SEQ ID NO:222), IFVIDDK (SEQ ID NO:223), IFVIDDKS (SEQ ID NO:224), IFVIDDKSG (SEQ ID NO:225), EEY, IEEY (SEQ ID NO:226), EEYT (SEQ ID NO:227), VIEEY (SEQ ID NO:228), IEEYT (SEQ ID NO:229), VIEEYT (SEQ ID NO:230), EEYTG (SEQ ID NO:231), IEEYTG (SEQ ID NO:232), VIEEYTG (SEQ ID NO:233), FVIEEY (SEQ ID NO:234), FVIEEYT (SEQ ID NO:235), FVIEEYTG (SEQ ID NO:236), FFVIEEY (SEQ ID NO:237), FFVIEEYT (SEQ ID NO:238), FFVIEEYTG (SEQ ID NO:239), EAQ, VEAQ (SEQ ID NO:240), EAQT (SEQ ID NO:241), SVEAQ (SEQ ID NO:242), VEAQT (SEQ ID NO:243), SVEAQT (SEQ ID NO:244), EAQTG (SEQ ID NO:245), VEAQTG (SEQ ID NO:246), SVEAQTG (SEQ ID NO:247), FSVEAQ (SEQ ID NO:248), FSVEAQT (SEQ ID NO:249), FSVEAQTG (SEQ ID NO:250), YFSVEAQ (SEQ ID NO:251), YFSVEAQT (SEQ ID NO:252) and YFSVEAQTG (SEQ ID NO:253); or (b) an analogue of any of the foregoing sequences that differs in one or more substitutions, deletions, additions and/or insertions such that that ability of the analogue to modulate an OB-cadherin-mediated function is not substantially diminished. For example, the agent may comprise a linear peptide having the sequence N-Ac-IFVIDDKSG-NH₂ (SEQ ID NO:225), N-Ac-FFVIEEYTG-NH₂ (SEQ ID NO:239) or N-Ac-YFSVEAQTG-NH₂ (SEQ ID NO:253). The OB-cadherin CAR sequence may, but need not, be present within a cyclic peptide.

Illustrative cadherin-5 (also known as VE-cadherin) antagonists can comprise: (a) one or more cadherin-5 CAR sequences selected from the group consisting of DAE, VDAE (SEQ ID NO:254), DAET (SEQ ID NO:255), RVDAE (SEQ ID NO:256), VDAET (SEQ ID NO:257), RVDAET (SEQ ID NO:258), DAETG (SEQ ID NO:259), VDAETG (SEQ ID NO:260), RVDAETG (SEQ ID NO:261), FRVDAE (SEQ ID NO:262), FRVDAET (SEQ ID NO:263), FRVDAETG (SEQ ID NO:264), VFRVDAE (SEQ ID NO:265), VFRVDAET (SEQ ID NO:266) and VFRVDAETG (SEQ ID NO:267); or (b) an analogue of any of the foregoing sequences that differs in one or more substitutions, deletions, additions and/or insertions such that that ability of the analogue to modulate a cadherin-5-mediated function is not substantially diminished. For example, the agent may comprise a linear peptide having the sequence N-Ac-VFRVDAETG-NH₂ (SEQ ID NO:267). The cadherin-5 CAR sequence may, but need not, be present within a cyclic peptide.

Anticancer Agents

As noted above, the present invention provides compositions and methods wherein compounds of the present invention are used in combination with other agents or treatment modalities. In certain embodiments, for example, one or more compounds of the invention are used in combination with one or more anticancer agents.

In one embodiment, anticancer agents used in combination with a compound of the invention may comprise anticancer alkylating agents, including, but not limited to: (1) nitrogen mustards (e.g., mechlorethamine, cyclophosphamide, ifosfamide, trofosfamide, melphalan (L-sarcolysin) and chlorambucil); (2) ethylenimines and methylmelamines (e.g., hexamethylmelamine and thiotepa); (3) alkyl sulfonates (e.g., busulfan); (4) nitrosoureas (e.g., carmustine (BCNU) and streptozocin (streptozotocin); (5) triazenes (e.g., dacarbazine (DTIC; dimethyltriazenoimid-azolecarboxamide) and temozolomide).

In another embodiment, anticancer antimetabolite agents are employed in combination with a compound of the invention. These may include, but are not limited to: (1) pyrimidine analogs (e.g., fluorouracil (5-fluorouracil; 5-FU) and floxuridine (fluoride-oxyuridine; FUdR); capecitabine, pemetrexed, cytarabine (cytosine arabinoside) and gemcitabine); (2) purine analogs and related inhibitors (e.g., mercaptopurine (6-mercaptopurine; 6-MP) and thioguanine) and/or (3) folic acid analogs (e.g., methotrexate).

Natural product-related anticancer agents may also be used in combination with cadherin antagonists according to the invention. These may include, but are not limited to: (1) vinca alkaloids (e.g., vinblastine (VLB) and vincristine); (2) taxanes (e.g., paclitaxel and docetaxel); (3) epipodophylltoxins (e.g., etoposide and teniposide); (4) camptothecins (e.g., topotecan and irinotecan); (5) antibiotics (e.g., dactinomycin (actinomycin D), daunorubicin (daunomycin; rubidomycin), doxorubicin, bleomycin, mitomycin (mitomycin C); and/or anthracycline agents (e.g., eiprubicin, idarubicin and liposomal doxorubicin). In a particular embodiment embodiment, the natural product-related anticancer agent is not a vinca alkaloid or paclitaxel.

In yet another embodiment, anticancer enzymes (e.g., l-asparaginase) and/or biological response modifiers or immunostimulators (e.g., interferon-alpha, interleukin-2 and other interleukins) may be used in combinations as described herein.

Still further anticancer agents which may be used in the combinations of the invention include, but are not limited to: (1) platinum-based anticancer agents such as platinum coordination complexes (e.g., cisplatin (cis-DDP), carboplatin and oxaliplatin); (2) anthracenediones (e.g., mitoxantrone); (3) methylhydrazine derivatives (e.g., procarbazine (N-methylhydrazine, MIH)); (4) adrenocortical suppressants (e.g., mitotane (o,p′-DD) and aminoglutethimide); (5) tyrosine kinase inhibitors (e.g., imatinib; erlotinib and gefitinib); and (6) multi-targeted kinase inhibitors (e.g., sunitinib; sorafanib and dasatinib).

Certain hormones and related antagonists may also be used according to the invention in combination with the compounds herein. These may include, but are not limited to: (1) adrenocorticosteriods (e.g., prednisone and prednisolone); (2) estrogens (e.g., diethylstilbestrol); (3) progestins (e.g., megestrol acetate); (4) aromatase inhibitors (e.g., exemestane and letrozole) and (5) antiestrogen (e.g., tamoxifen).

Anticancer antibodies are also useful in combinations of the invention. These may include, but are not limited to: (1) anti-angiogenesis antibodies (e.g., bevacizumab); (2) anti-CD20 antibodies (e.g., rituximab); (3) anti-epidermal growth factor receptor antibodies (e.g., cetuximab and panitumomab; and (4) radiolabelled antibodies (e.g., ¹³¹I-tositumomab).

In another embodiment, radiation therapy may be used in combination with the compounds herein including, for example, external beam therapy, implanted pellets, and other conventional radiation treatment methodologies.

It will be understood on the part of the skilled artisan, in view of this disclosure, that there exist a multitude of formulation, dosing and administration strategies that can be used to achieve an improved therapeutic benefit when using the compositions and methods described herein. Particular formulation components, dosing concentrations and/or administration schedules useful for a given agent or combination of agents, while still achieving the therapeutic benefits described herein, may be routinely identified using skills and techniques known and established in the art. Accordingly, all such components, concentrations and/or schedules are considered within the spirit and scope of the present invention.

Compounds, alone or in combination, are administered to a subject or patient in need thereof in a manner appropriate to the condition to be treated. The subject or patient can be essentially any mammal such as a cancer-bearing dog, cat or human. Appropriate dosages, timing, duration and frequency of administration will be determined by such factors as the condition of the patient, the type and severity of the patient's disease and the method of administration. In general, an appropriate dosage and treatment regimen provides the agent(s) in an amount sufficient to achieve an improved therapeutic benefit, as described herein, relative to the separate components administered individually.

Optimal dosages for a given compound or combination in the context of a given indication may generally be determined using experimental models and/or clinical trials. In general, the use of the minimum dosage that is sufficient to provide effective therapy is preferred. Patients may generally be monitored for therapeutic effectiveness using assays suitable for the condition being treated or prevented, which will be familiar to those of ordinary skill in the art.

Suitable concentration/dosage ranges used for many known therapeutics, are well known, and, when used in combination with one or more compounds of the invention, will generally be within these same established and accepted ranges. Typically, the concentration of a compound used in the methods of the invention will be at or below the maximum tolerated dose for the agent that is being used and/or at or below the typical dose when the agents are administered individually.

The route of administration for a compound of the invention may vary depending on the particular agent used, and specific delivery or administration routes are not critical provided that acceptable exposure of a compound or compounds to a tissue or site of interest is achieved. Suitable delivery routes for the agents described herein are indeed well known and established and any such routes may be used in according with the invention. In many embodiments, compounds of the invention may be administered systemically, such as intravenously. Anticancer agents used in combination with one or more compounds of the invention will generally be administered by their conventional and/or preferred routes and schedules of administration. Further, alternative administration schedules and strategies preferred for a given combination, and indication, may be identified and implemented by a skilled artisan using routine and standard methodologies.

The following examples are provided for purposes of illustration, not limitation.

### EXAMPLES

Referring to the examples that follow, compounds of the present invention were synthesized using the methods described herein, or other methods, which are well known in the art. It should be evident to those skilled in the art that appropriate substitution of both the materials and methods disclosed herein will produce the examples illustrated below and those encompassed by the scope of the invention.

All temperatures are given in degrees Centigrade. Reagents were purchased from commercial sources or prepared following literature procedures. Unless otherwise noted, reactions were carried out under a positive pressure of nitrogen. Reaction vessels were sealed with rubber septa or Teflon screw caps. Nitrogen was introduced through Tygon tubing, fitted with a large bore syringe needle. Concentration under vacuum refers to the removal of solvent on a Büchi Rotary Evaporator.

Analytical high performance liquid chromatography (HPLC) was performed using a Supelco discovery C₁₈ 15 cm×4.6 mm/5 μm column coupled with an Agilent 1050 series VWD UV detector at 210 nm. Conditions: Solvent A: H₂O/1% acetonitrile/0.1% HCO₂H; Solvent B: methanol. HPLC purification was performed using a 50 mm Varian Dynamax HPLC 21.4 mm Microsorb Guard-8 C₁₈ column, Dyonex Chromeleon operating system coupled with a Varian Prostar 320 UV-vis detector (210 nm) and a Sedex55 ELS detector. Conditions: Solvent A: H₂O; Solvent B: Acetonitrile/0.1% TFA. The appropriate solvent gradient for purification was determined based on the results of analytical HPLC experiments. The resulting fractions were analyzed, combined as appropriate, and evaporated under reduced pressure to provide purified material.

Proton nuclear magnetic resonance (¹H NMR) spectra were recorded on either a Varian INOVA 400 MHz (¹H) NMR spectrometer, Varian INOVA 500 MHz (¹H) NMR spectrometer, Bruker ARX 300 MHz (¹H) NMR spectrometer, Bruker DPX 400 MHz (¹H) NMR spectrometer, or a Bruker DRX 500 MHz (1H) NMR spectrometer. All spectra were determined in the solvents indicated. Although chemical shifts are reported in ppm downfield of tetramethylsilane, they are referenced to the residual proton peak of the respective solvent peak for ¹H NMR. Interproton coupling constants are reported in Hertz (Hz).

Liquid chromatography/mass spectrometry (LCMS) spectra were obtained using a ThermoFinnigan AQA MS ESI instrument utilizing a Phenomenex Aqua 5 micron C₁₈ 125 Å 50×4.60 mm column. The spray setting for the MS probe was at 350 μL/min with a cone voltage at 25 mV and a probe temperature at 450° C. The LC spectra were recorded using ELS (Evaporating Light Scattering) detection.

Microwave reactions were carried out on a CEM Discover® Microwave Synthesis System, fitted with a CEM Explorer® Automated Synthesis Workstation. The magnetron frequency was 2450 MHz with a maximum power output of 300 W and a circular single-mode self-tuning microwave applicator. Reactions were carried out in sealed disposable 10 mL glass microwave vessels with variable speed magnetic stirring. Internal pressure was maintained below 20 Bar. P_{Max} refers to irradiation of a reaction at maximum power with concomitant forced-air cooling to maintain the specified reaction temperature.

Silica gel chromatography was carried out on a Teledyne ISCO CombiFlash Companion Flash Chromatography System with a variable flow rate from 5-100 mL/min. The columns used were Teledyne ISCORediSep Disposable Flash Columns (4, 12, 40, 80, or 120 g prepacked silica gel), which were run with a maximum capacity of 1 g crude sample per 10 g silica gel. Samples were preloaded on Celite in Analogix Sample Loading Cartridges with frits (1/in, 1/out). Peaks were detected by variable wavelength UV absorption (200-360 nm). The resulting fractions were analyzed, combined as appropriate, and evaporated under reduced pressure to provide purified material.

### EXAMPLE 1

### 3-(4-TERT-BUTYLPHENYL)-5-ETHYL-4H-[1,2,4]TRIAZOLE

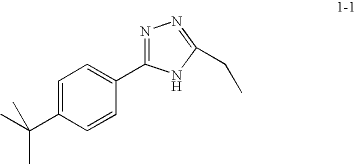

To a disposable glass microwave reactor vessel (10 mL) was added tert-butylbenzhydrazide (37 mg, 0.19 mmol), propionitrile (0.2 mL, 2.8 mmol), potassium carbonate (13 mg, 0.1 mmol), and n-butanol (1 mL). The reaction was stirred under microwave irradiation (P_{Max}, 150° C., 250 W) for 25 minutes. The solution was concentrated to dryness under vacuum, and the resultant mixture was purified by preparative HPLC. Calculated for C₁₄H₁₉N₃; 229. Observed; 229 (M+H)⁺. ¹H NMR (400 MHz, CHLOROFORM-d) δ ppm 1.25-1.33 (m, 12H) 2.76 (q, J=7.6 Hz, 2H) 7.38 (d, J=8.4 Hz, 2H) 7.90 (d, J=8.4 Hz, 2H).

### EXAMPLE 2

### 3-METHYL-5-NAPHTHALEN-2-YL-4H-[1,2,4]TRIAZOLE

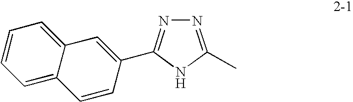

Compound 2-1 was prepared from the appropriate nitrile and hydrazide in a manner analogous to that described for compound 1-1. Calculated for C₁₃H₁₁N₃; 209. Observed; 210 (M+H)⁺. ¹H NMR (400 MHz, CHLOROFORM-d) δ ppm 2.59 (s, 3H) 7.41-7.62 (m, 2H) 7.83-7.89 (m, 1H) 7.91 (s, 2H) 8.15 (dd, J=8.6, 1.7 Hz, 2H) 8.57 (s, 1H).

### EXAMPLE 3

### 3-METHYL-5-PHENYL-4H-[1,2,4]TRIAZOLE

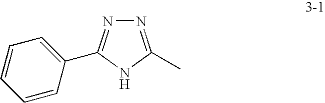

Compound 3-1 was prepared from the appropriate nitrile and hydrazide in a manner analogous to that described for compound 1-1. Calculated for C₉H₉N₃; 159. Observed; 160 (M+H)⁺. ¹H NMR (400 MHz, CHLOROFORM-d) δ ppm 2.51 (s, 3H) 7.31-7.56 (m, 3H) 7.90-8.11 (m, 2H).

### EXAMPLE 4

### 3-METHYL-5-O-TOLYL-4H-[1,2,4]TRIAZOLE

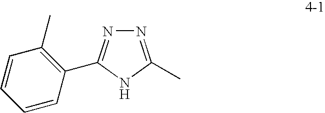

Compound 4-1 was prepared from the appropriate nitrile and hydrazide in a manner analogous to that described for compound 1-1. Calculated for C₁₀H₁₁N₃; 173. Observed; 174 (M+H)⁺. ¹H NMR (400 MHz, CHLOROFORM-d) δ ppm 2.48 (s, 3H) 2.55 (s, 3H) 7.16-7.39 (m, 3H) 7.74 (d, J=7.51 Hz, 1H).

### EXAMPLE 5

### 3-METHYL-5-M-TOLYL-4H-[1,2,4]TRIAZOLE

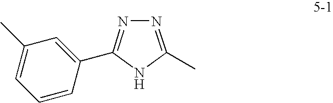

Compound 5-1 was prepared from the appropriate nitrile and hydrazide in a manner analogous to that described for compound 1-1. Calculated for C₁₀H₁₁N₃; 173. Observed; 174 (M+H)⁺. ¹H NMR (400 MHz, CHLOROFORM-d) δ ppm 2.39 (s, 3H) 2.52 (s, 3H) 7.16-7.41 (m, 2H) 7.74-7.92 (m, 2H).

### EXAMPLE 6

### 3-METHYL-5-P-TOLYL-4H-[1,2,4]TRIAZOLE

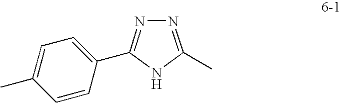

Compound 6-1 was prepared from the appropriate nitrile and hydrazide in a manner analogous to that described for compound 1-1. Calculated for C₁₀H₁₁N₃; 173. Observed; 174 (M+H)⁺. ¹H NMR (400 MHz, CHLOROFORM-d) δ ppm 2.38 (s, 3H) 2.48 (s, 3H) 7.21 (d, J=7.88 Hz, 2H) 7.88 (d, J=7.96 Hz, 2H).

### EXAMPLE 7

### 3-(2-CHLOROPHENYL)-5-METHYL-4H-[1,2,4]TRIAZOLE

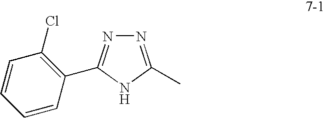

Compound 7-1 was prepared from the appropriate nitrile and hydrazide in a manner analogous to that described for compound 1-1. Calculated for C₉H₈ClN₃; 193. Observed; 194 (M+H)⁺. ¹H NMR (400 MHz, CHLOROFORM-d) δ ppm 2.52 (s, 3H) 7.30-7.45 (m, 2H) 7.44-7.58 (m, 1H) 8.04-8.22 (m, 1H).

### EXAMPLE 8

### 3-(3-CHLOROPHENYL)-5-METHYL-4H-[1,2,4]TRIAZOLE

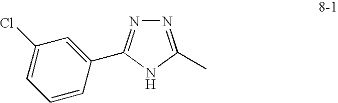

Compound 8-1 was prepared from the appropriate nitrile and hydrazide in a manner analogous to that described for compound 1-1. Calculated for C₉H₈ClN₃; 193. Observed; 194 (M+H)⁺. ¹H NMR (400 MHz, CHLOROFORM-d) δ ppm 2.53 (s, 3H) 7.29-7.45 (m, 2H) 7.91 (d, J=6.66 Hz, 1H) 8.05 (s, 1H).

### EXAMPLE 9

### 3-(4-CHLOROPHENYL)-5-METHYL-4H-[1,2,4]TRIAZOLE

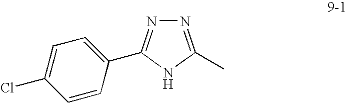

Compound 9-1 was prepared from the appropriate nitrile and hydrazide in a manner analogous to that described for compound 1-1. Calculated for C₉H₈ClN₃; 193. Observed; 194 (M+H)⁺. ¹H NMR (400 MHz, CHLOROFORM-d) δ ppm 2.55 (s, 3H) 7.42 (d, J=8.43 Hz, 2H) 7.99 (d, J=8.43 Hz, 2H).

### EXAMPLE 10

### 3-BENZYL-5-METHYL-4H-[1,2,4]TRIAZOLE

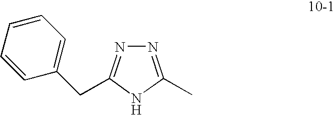

Compound 10-1 was prepared from the appropriate nitrile and hydrazide in a manner analogous to that described for compound 1-1. Calculated for C₁₀H₁₁N₃; 173. Observed; 174 (M+H)⁺. ¹H NMR (400 MHz, CHLOROFORM-d) δ ppm 2.38 (s, 3H) 4.06 (s, 2H) 7.21-7.33 (m, 5H).

### EXAMPLE 11

### 3-(3-METHOXYPHENYL)-5-METHYL-4H-[1,2,4]TRIAZOLE

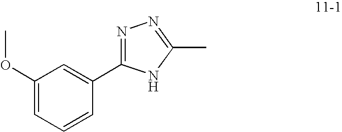

Compound 11-1 was prepared from the appropriate nitrile and hydrazide in a manner analogous to that described for compound 1-1. Calculated for C₁₀H₁₁N₃O; 189. Observed; 190 (M+H)⁺. ¹H NMR (400 MHz, CHLOROFORM-d) δ ppm 2.52 (s, 3H) 3.85 (s, 3H) 6.97 (dd, J=8.15, 1.78 Hz, 1H) 7.34 (t, J=7.92 Hz, 1H) 7.50-7.68 (m, 2H).

### EXAMPLE 12

### 3-(4-METHOXYPHENYL)-5-METHYL-4H-[1,2,4]TRIAZOLE

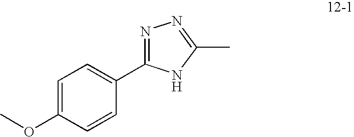

Compound 12-1 was prepared from the appropriate nitrile and hydrazide in a manner analogous to that described for compound 1-1. Calculated for C₁₀H₁₁N₃O; 189. Observed; 190 (M+H)⁺. ¹H NMR (400 MHz, CHLOROFORM-d) δ ppm 2.49 (s, 3H) 3.84 (s, 3H) 6.93 (d, J=8.93 Hz, 2H) 7.93 (d, J=8.94 Hz, 2H).

### EXAMPLE 13

### 3-METHYL-5-(4-PHENOXYPHENYL)-4H-[1,2,4]TRIAZOLE

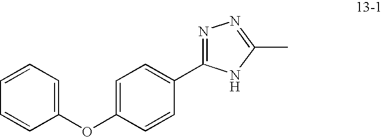

Compound 13-1 was prepared from the appropriate nitrile and hydrazide in a manner analogous to that described for compound 1-1. Calculated for C₁₅H₁₃N₃O; 251. Observed; 252 (M+H)⁺. ¹H NMR (400 MHz, CHLOROFORM-d) δ ppm 2.53 (s, 3H) 7.06 (dd, J=8.33, 2.81 Hz, 4H) 7.15 (t, J=7.35 Hz, 1H) 7.37 (t, J=7.85 Hz, 2H) 7.99 (d, J=8.61 Hz, 2H).

### EXAMPLE 14

### 3-(3,4-DICHLOROPHENYL)-5-METHYL-4H-[1,2,4]TRIAZOLE

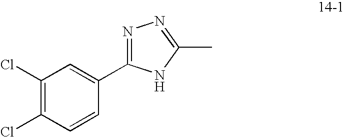

Compound 14-1 was prepared from the appropriate nitrile and hydrazide in a manner analogous to that described for compound 1-1. Calculated for C₉H₇Cl₂N₃; 228. Observed; 229 (M+H)⁺. ¹H NMR (400 MHz, METHANOL-d4) δ ppm 7.62 (d, J=8.39 Hz, 1H) 7.91 (dd, J=8.37, 1.56 Hz, 1H) 8.14 (d, J=1.53 Hz, 1H).

### EXAMPLE 15

### 3-BIPHENYL-4-YL-5-METHYL-4H-[1,2,4]TRIAZOLE

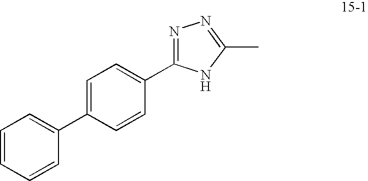

Compound 15-1 was prepared from the appropriate nitrile and hydrazide in a manner analogous to that described for compound 1-1. Calculated for C₁₅H₁₃N₃; 235.

Observed; 236 (M+H)⁺. ¹H NMR (400 MHz, CHLOROFORM-d) δ ppm 2.56 (s, 3H) 7.38 (d, J=7.26 Hz, 1H) 7.46 (t, J=7.47 Hz, 2H) 7.66 (dd, J=18.32, 7.81 Hz, 4H) 8.11 (d, J=8.19 Hz, 2H).

### EXAMPLE 16

### 3-METHYL-5-(3-PHENOXYPHENYL)-4H-[1,2,4]TRIAZOLE

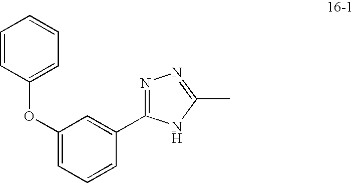

Compound 16-1 was prepared from the appropriate nitrile and hydrazide in a manner analogous to that described for compound 1-1. Calculated for C₁₅H₁₃N₃O; 251. Observed; 252 (M+H)⁺. ¹H NMR (400 MHz, CHLOROFORM-d) δ ppm 2.47 (s, 3H) 6.96-7.14 (m, 4H) 7.31 (t, J=7.8 Hz, 2H) 7.39 (t, J=7.9 Hz, 1H) 7.67 (s, 1H) 7.76 (d, J=7.6 Hz, 1H).

### EXAMPLE 17

### 3-(2,4-DICHLOROPHENYL)-5-METHYL-4H-[1,2,4]TRIAZOLE

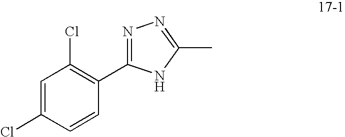

Compound 17-1 was prepared from the appropriate nitrile and hydrazide in a manner analogous to that described for compound 1-1. Calculated for C₉H₇Cl₂N₃; 228. Observed; 229 (M+H)⁺. ¹H NMR (400 MHz, CHLOROFORM-d) δ ppm 2.52 (s, 3H) 7.36 (dd, J=8.4, 1.7 Hz, 1H) 7.51 (d, J=1.6 Hz, 1H) 8.02 (d, J=8.4 Hz, 1H).

### EXAMPLE 18

### 3-HEPTYL-5-METHYL-4H-[1,2,4]TRIAZOLE

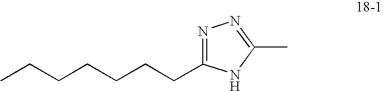

Compound 18-1 was prepared from the appropriate nitrile and hydrazide in a manner analogous to that described for compound 1-1. Calculated for C₁₀H₁₉N₃; 181. Observed; 182 (M+H)⁺. ¹H NMR (400 MHz, METHANOL-d4) δ ppm 0.91 (t, J=6.5 Hz, 3H) 1.33 (d, J=10.7 Hz, 9H) 1.72 (t, 2H) 2.37 (s, 3H) 2.70 (t, J=7.6 Hz, 2H).

### EXAMPLE 19

### 3-(4-TERT-BUTYLPHENYL)-5-PROPYL-4H-[1,2,4]TRIAZOLE

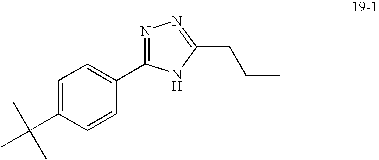

Compound 19-1 was prepared from the appropriate nitrile and hydrazide in a manner analogous to that described for compound 1-1. Calculated for C₁₅H₂₁N₃; 243. Observed; 244 (M+H)⁺. ¹H NMR (400 MHz, METHANOL-d4) δ ppm 1.03 (t, J=7.4 Hz, 3H) 1.37 (s, 9H) 1.77-1.90 (m, 2H) 2.82 (t, J=7.5 Hz, 2H) 7.54 (d, J=8.4 Hz, 2H) 7.90 (d, J=8.1 Hz, 2H).

### EXAMPLE 20

### 3-BUTYL-5-(4-TERT-BUTYLPHENYL)-4H-[1,2,4]TRIAZOLE

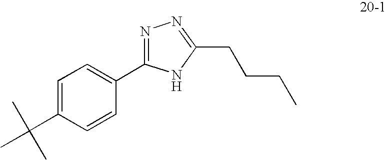

Compound 20-1 was prepared from the appropriate nitrile and hydrazide in a manner analogous to that described for compound 1-1. Calculated for C₁₆H₂₃N₃; 257. Observed; 258 (M+H)⁺. ¹H NMR (400 MHz, METHANOL-d4) δ ppm 1.00 (t, J=7.4 Hz, 3 H) 1.38 (s, 9H) 1.40-1.51 (m, 2H) 1.73-1.87 (m, 2H) 2.85 (t, J=7.7 Hz, 2H) 7.55 (d, J=8.4 Hz, 2H) 7.91 (d, J=8.3 Hz, 2H).

### EXAMPLE 21

### 3-(4-TERT-BUTYLPHENYL)-5-ISOPROPYL-4H-[1,2,4]TRIAZOLE

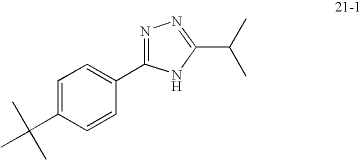

Compound 21-1 was prepared from the appropriate nitrile and hydrazide in a manner analogous to that described for compound 1-1. Calculated for C₁₅H₂₁N₃; 243. Observed; 244 (M+H)⁺. ¹H NMR (400 MHz, METHANOL-d4) δ ppm 1.38 (s, 9H) 1.41 (d, J=6.9 Hz, 6H) 3.09-3.29 (m, 1H) 7.54 (d, 2H) 7.91 (d, 2H).

### EXAMPLE 22

### 3-(4-TERT-BUTYLPHENYL)-5-CYCLOPROPYL-4H-[1,2,4]TRIAZOLE

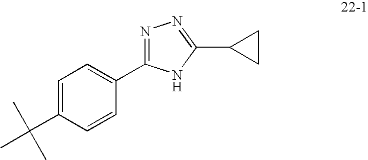

Compound 22-1 was prepared from the appropriate nitrile and hydrazide in a manner analogous to that described for compound 1-1. Calculated for C₁₅H₁₉N₃; 241. Observed; 242 (M+H)⁺. ¹H NMR (400 MHz, METHANOL-d4) δ ppm 1.07 (s, 4H) 1.37 (s, 9H) 2.03-2.17 (m, J=5.0 Hz, 1H) 7.52 (d, J=7.5 Hz, 2H) 7.88 (d, J=7.9 Hz, 2H).

### EXAMPLE 23

### 3-(4-TERT-BUTYLPHENYL)-5-CYCLOHEXYL-4H-[1,2,4]TRIAZOLE

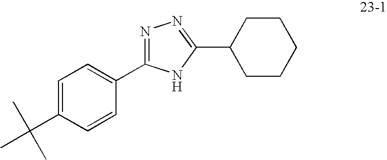

Compound 23-1 was prepared from the appropriate nitrile and hydrazide in a manner analogous to that described for compound 1-1. Calculated for C₁₈H₂₅N₃; 283. Observed; 284 (M+H)⁺. ¹H NMR (400 MHz, METHANOL-d4) δ ppm 1.38 (s, 9H) 1.42-1.57 (m, 2H) 1.59-1.73 (m, 2H) 1.81 (d, J=12.2 Hz, 1H) 1.91 (d, J=12.9 Hz, 2H) 2.08 (d, J=12.0 Hz, 2H) 2.89 (t, 1H) 7.54 (d, J=8.3 Hz, 2H) 7.92 (d, J=8.3 Hz, 2H).

### EXAMPLE 24

### 3-(4-TERT-BUTYLPHENYL)-5-PHENYL-4H-[1,2,4]TRIAZOLE

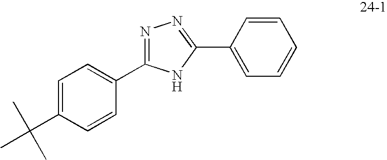

Compound 24-1 was prepared from the appropriate nitrile and hydrazide in a manner analogous to that described for compound 1-1. Calculated for C₁₈H₁₉N₃; 277. Observed; 278 (M+H)⁺. ¹H NMR (400 MHz, METHANOL-d4) δ ppm 1.40 (s, 9H) 7.56 (t, 5H) 8.05 (t, 4H).

### EXAMPLE 25

### 3-(4-TERT-BUTYLPHENYL)-5-CYCLOBUTYL-4H-[1,2,4]TRIAZOLE

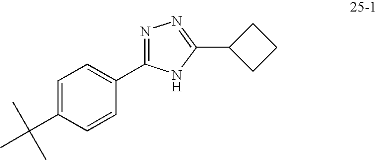

Compound 25-1 was prepared from the appropriate nitrile and hydrazide in a manner analogous to that described for compound 1-1. Calculated for C₁₆H₂₁N₃; 255. Observed; 256 (M+H)⁺. ¹H NMR (400 MHz, METHANOL-d4) δ ppm 1.37 (s, 9H) 1.92-2.08 (m, 1H) 2.07-2.32 (m, 1H) 2.45 (q, J=8.6 Hz, 4H) 3.62-3.84 (m, 1H) 7.53 (d, J=8.3 Hz, 2H) 7.92 (d, J=8.3 Hz, 2H).

### EXAMPLE 26

### 3-BENZYL-5-(4-TERT-BUTYLPHENYL)-4H-[1,2,4]TRIAZOLE

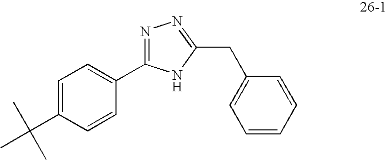

Compound 26-1 was prepared from the appropriate nitrile and hydrazide in a manner analogous to that described for compound 1-1. Calculated for C₁₉H₂₁N₃; 291. Observed; 292 (M+H)⁺. ¹H NMR (400 MHz, METHANOL-d4) δ ppm 1.36 (s, 9H) 4.16 (s, 2H) 7.18-7.28 (m, 1H) 7.27-7.36 (m, 4H) 7.53 (d, J=8.3 Hz, 2H) 7.90 (d, J=8.3 Hz, 2H).

### EXAMPLE 27

### 4-[5-(4-TERT-BUTYLPHENYL)-4H-[1,2,4]TRIAZOLE-3-YL]-PYRIDINE

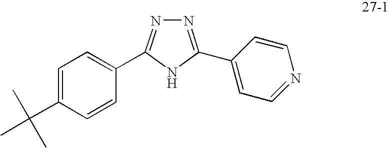

Compound 27-1 was prepared from the appropriate nitrile and hydrazide in a manner analogous to that described for compound 1-1. Calculated for C₁₇H₁₈N₄; 278. Observed; 279 (M+H)⁺. ¹H NMR (400 MHz, METHANOL-d4) δ ppm 1.37 (s, 9H) 7.97 (d, J=8.3 Hz, 2H) 8.11 (d, J=5.5 Hz, 2H) 8.66 (d, J=5.1 Hz, 2H).

### EXAMPLE 28

### 2-[5-(4-TERT-BUTYLPHENYL)-4H-[1,2,4]TRIAZOLE-3-YL]-PYRAZINE

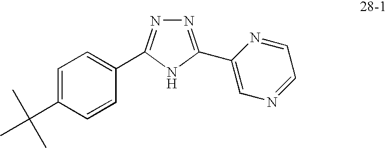

Compound 28-1 was prepared from the appropriate nitrile and hydrazide in a manner analogous to that described for compound 1-1. Calculated for C₁₆H₁₇N₅; 279. Observed; 280 (M+H)⁺. ¹H NMR (400 MHz, METHANOL-d4) δ ppm 1.32-1.47 (m, 9H) 7.61 (d, J=8.3 Hz, 2H) 8.05 (d, J=8.3 Hz, 2H) 8.70 (d, J=2.2 Hz, 1H) 8.76 (s, 1H) 9.42 (s, 1H).

### EXAMPLE 29

### DIMETHYL-[4-(5-METHYL-4H-[1,2,4]TRIAZOLE-3-YL)-PHENYL]-AMINE

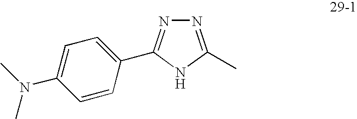

Compound 29-1 was prepared from the appropriate nitrile and hydrazide in a manner analogous to that described for compound 1-1. Calculated for CIH₁₄N₄; 202. Observed; 203 (M+H)⁺. ¹H NMR (400 MHz, METHANOL-d4) δ ppm 2.42 (s, 3H) 3.01 (s, 6H) 6.81 (d, J=8.8 Hz, 2H) 7.78 (d, J=8.6 Hz, 2H).

### EXAMPLE 30

### 3-(4-BENZYLOXYPHENYL)-5-METHYL-4H-[1,2,4]TRIAZOLE

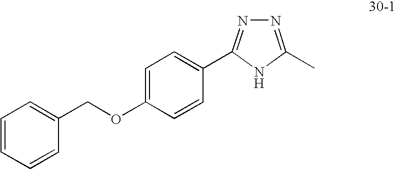

Compound 30-1 was prepared from the appropriate nitrile and hydrazide in a manner analogous to that described for compound 1-1. Calculated for C₁₆H₁₅N₃O; 265. Observed; 266 (M+H)⁺. ¹H NMR (400 MHz, METHANOL-d4) δ ppm 2.47 (s, 3H) 5.17 (s, 2H) 7.11 (d, J=8.6 Hz, 2H) 7.30-7.37 (m, 1H) 7.40 (t, J=7.4 Hz, 2H) 7.47 (d, 2H) 7.90 (d, J=8.5 Hz, 2H).

### EXAMPLE 31

### 3-(4-ISOPROPYLPHENYL)-5-METHYL-4H-[1,2,4]TRIAZOLE

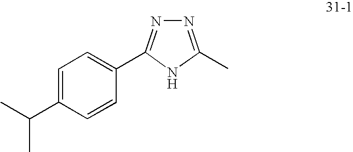

Compound 31-1 was prepared from the appropriate nitrile and hydrazide in a manner analogous to that described for compound 1-1. Calculated for C₁₂H₁₅N₃; 201. Observed; 201 (M+H)⁺. ¹H NMR (400 MHz, CHLOROFORM-d) δ ppm 1.29 (d, J=6.9 Hz, 6H) 2.54 (s, 3H) 2.74-3.13 (m, 1H) 7.32 (d, J=8.4 Hz, 2H) 7.94 (d, J=8.4 Hz, 2H).

### EXAMPLE 32

### 3-(4-BUTOXYPHENYL)-5-METHYL-4H-[1,2,4]TRIAZOLE

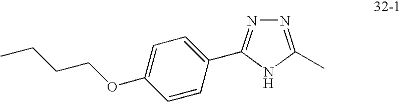

Compound 32-1 was prepared from the appropriate nitrile and hydrazide in a manner analogous to that described for compound 1-1. Calculated for C₁₃H₁₇N₃O; 231. Observed; 232 (M+H)⁺. ¹H NMR (400 MHz, METHANOL-d4) δ ppm 0.99 (t, J=7.4 Hz, 3H) 1.46-1.58 (m, 2H) 1.72-1.83 (m, 2H) 1.92 (s, 1H) 2.44 (s, 3H) 4.02 (t, J=6.4 Hz, 2H) 6.99 (d, J=8.5 Hz, 2H) 7.85 (d, J=8.6 Hz, 2H).

### EXAMPLE 33

### 2-[5-(4-TERT-BUTYLPHENYL)-4H-[1,2,4]TRIAZOLE-3-YL]-PYRIDINE

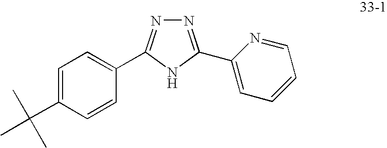

Compound 33-1 was prepared from the appropriate nitrile and hydrazide in a manner analogous to that described for compound 1-1. Calculated for C₁₇H₁₈N₄; 278. Observed; 279 (M+H)⁺. ¹H NMR (400 MHz, METHANOL-d4) δ ppm 1.33-1.42 (m, 9H) 7.50-7.65 (m, 5H) 7.91 (d, 1H) 7.96 (d, 1H) 8.03 (d, J=8.2 Hz, 3H) 8.57 (d, J=7.6 Hz, 1H).

### EXAMPLE 34

### 3-[5-(4-TERT-BUTYLPHENYL)-4H-[1,2,4]TRIAZOLE-3-YL]-PYRIDINE

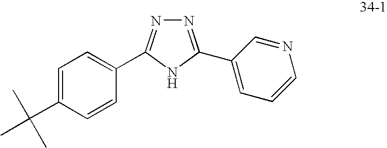

Compound 34-1 was prepared from the appropriate nitrile and hydrazide in a manner analogous to that described for compound 1-1. Calculated for C₁₇H₁₈N₄; 278. Observed; 279 (M+H)⁺. ¹H NMR (400 MHz, METHANOL-d4) δ ppm 1.39 (s, 9H) 7.50-7.68 (m, 3H) 7.99 (d, J=8.3 Hz, 2H) 8.52 (d, J=7.9 Hz, 1H) 8.62 (s, 1H) 9.28 (s, 1H).

### EXAMPLE 35

### 3-METHYL-5-NAPHTHALEN-1-YL-4H-[1,2,4]TRIAZOLE

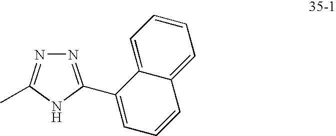

Compound 35-1 was prepared from the appropriate nitrile and hydrazide in a manner analogous to that described for compound 1-1. Calculated for C₁₃H₁₁N₃; 209. Observed; 210 (M+H)⁺. ¹H NMR (400 MHz, METHANOL-d4) δ ppm 2.57 (s, 3H) 7.50-7.63 (m, 3H) 7.87 (d, J=6.9 Hz, 1H) 7.92-7.99 (m, 1H) 8.01 (d, J=8.2 Hz, 1H) 8.55 (s, 1H).

### EXAMPLE 36

### 2-[4-(5-METHYL-4H-[1,2,4]TRIAZOLE-3-YL)-PHENYL]-PROPAN-2-OL

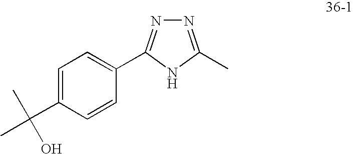

Compound 36-1 was prepared from the appropriate nitrile and hydrazide in a manner analogous to that described for compound 1-1. Calculated for C₁₂H₁₅N₃O; 217. Observed; 218 (M+H)⁺. ¹H NMR (400 MHz, METHANOL-d4) δ ppm 1.57 (s, 6H) 2.49 (s, 3H) 7.61 (d, J=8.2 Hz, 2H) 7.94 (d, J=8.2 Hz, 2H).

### EXAMPLE 37

### 3-SEC-BUTYL-5-(4-TERT-BUTYLPHENYL)-4H-[1,2,4]TRIAZOLE

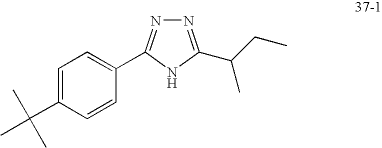

Compound 37-1 was prepared from the appropriate nitrile and hydrazide in a manner analogous to that described for compound 1-1. Calculated for C₁₆H₂₃N₃; 257. Observed; 258 (M+H)⁺. ¹H NMR (400 MHz, METHANOL-d4) δ ppm 0.94 (t, J=7.4 Hz, 3H) 1.36 (s, 9H) 1.40 (d, J=7.0 Hz, 3H) 1.68-1.93 (m, 2H) 2.96-3.08 (m, 1H) 7.56 (d, J=8.3 Hz, 2H) 7.91 (d, J=8.3 Hz, 2H).

### EXAMPLE 38

### 3-TERT-BUTYL-5-(4-TERT-BUTYLPHENYL)-4H-[1,2,4]TRIAZOLE

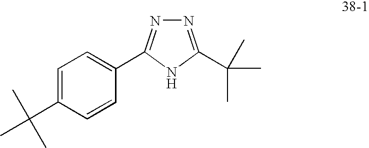

Compound 38-1 was prepared from the appropriate nitrile and hydrazide in a manner analogous to that described for compound 1-1. Calculated for C₁₆H₂₃N₃; 257. Observed; 258 (M+H)⁺. ¹H NMR (400 MHz, METHANOL-d4) δ ppm 1.38 (s, 9H) 1.47 (s, 9H) 7.72 (dd, 4H).

### EXAMPLE 39

### 3-BIPHENYL-4-YL-5-(4-TERT-BUTYLPHENYL)-4H-[1,2,4]TRIAZOLE

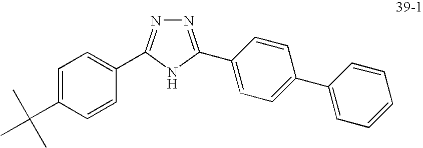

Compound 39-1 was prepared from the appropriate nitrile and hydrazide in a manner analogous to that described for compound 1-1. Calculated for C₂₄H₂₃N₃; 353. Observed; 354 (M+H)⁺. ¹H NMR (400 MHz, DMSO-d₆) δ ppm 1.33 (s, 9H) 7.37-7.44 (m, 1H) 7.50 (t, 2H) 7.56 (d, J=8.1 Hz, 2H) 7.75 (d, J=7.5 Hz, 2H) 7.84 (d, J=8.0 Hz, 2H) 8.02 (d, J=8.3 Hz, 2H) 8.17 (d, J=8.2 Hz, 2H).

### EXAMPLE 40

### 3-(4-TERT-BUTYLPHENYL)-5-NAPHTHALEN-1-YL-4H-[1,2,4]TRIAZOLE

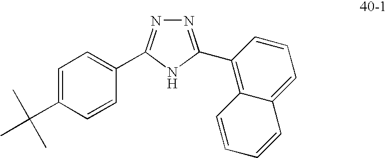

Compound 40-1 was prepared from the appropriate nitrile and hydrazide in a manner analogous to that described for compound 1-1. Calculated for C₂₂H₂₁N₃; 327. Observed; 328 (M+H)⁺. ¹H NMR (400 MHz, METHANOL-d4) δ ppm 1.33-1.42 (m, 9H) 7.50-7.65 (m, 5H) 7.91 (d, 1H) 7.96 (d, 1H) 8.03 (d, J=8.2 Hz, 3H) 8.57 (d, J=7.6 Hz, 1H).

### EXAMPLE 41

### 3-(4-TERT-BUTYLPHENYL)-5-(1H-IMIDAZOL-4-YLMETHYL)-4H-[1,2,4]TRIAZOLE

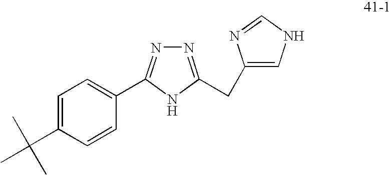

Compound 41-1 was prepared from the appropriate nitrile and hydrazide in a manner analogous to that described for compound 1-1. Calculated for C₁₆H₁₉N₅; 281. Observed; 282 (M+H)⁺. ¹H NMR (400 MHz, METHANOL-d4) δ ppm 1.34 (s, 9H) 4.14 (s, 2H) 6.97 (s, 1H) 7.51 (d, J=8.3 Hz, 2H) 7.68 (s, 1H) 7.88 (d, J=8.3 Hz, 2H).

### EXAMPLE 42

### DIETHYL-[4-(5-METHYL-4H-[1,2,4]TRIAZOLE-3-YL)-PHENYL]-AMINE

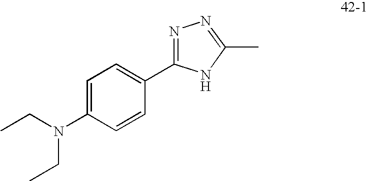

Compound 42-1 was prepared from the appropriate nitrile and hydrazide in a manner analogous to that described for compound 1-1. Calculated for C₁₃H₁₈N₄; 230. Observed; 231 (M+H)⁺. ¹H NMR (400 MHz, METHANOL-d4) δ ppm 1.18 (t, J=7.0 Hz, 6H) 2.42 (s, 3H) 3.44 (q, J=7.0 Hz, 4H) 6.75 (d, J=8.8 Hz, 2H) 7.74 (d, J=8.7 Hz, 2H).

### EXAMPLE 43

### 3-METHYL-5-NAPHTHALEN-1-YLMETHYL-4H-[1,2,4]TRIAZOLE

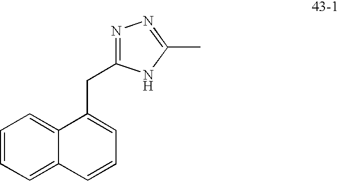

To a disposable glass microwave reactor vessel was added S-methylisothioamide hydroiodide (150 mg, 0.69 mmol), triethylamine (0.3 mL, 2.1 mmol), ammonium acetate (534 mg, 6.9 mmol), silica gel (450 mg), and 1-naphthyleneacethydrazide (140 mg, 0.69 mmol) The solution was stirred under microwave irradiation (P_{Max}, 120° C., 300 W) for 10 minutes. The solution was concentrated to dryness under vacuum, and the product was purified by silica gel chromatography (0-10% methanol in dichloromethane, linear gradient). The title compound was isolated following purification by preparative HPLC. Calculated for C₁₄H₁₃N₃; 223. Observed; 224 (M+H)⁺. ¹H NMR (400 MHz, CHLOROFORM-d) δ ppm 2.37 (s, 3H) 4.51 (s, 2H) 7.40-7.45 (m, 2H) 7.46-7.54 (m, 2H) 7.80 (dd, J=5.7, 3.7 Hz, 1H) 7.83-7.92 (m, J=6.7, 2.8 Hz, 1H) 7.96-8.06 (m, 1H).

### EXAMPLE 44

### 3-(2-METHOXYPHENYL)-5-METHYL-4H-[1,2,4]TRIAZOLE

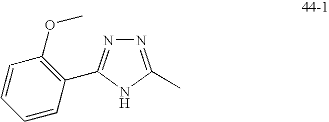

Compound 44-1 was prepared from the appropriate hydrazide in a manner analogous to that described for compound 43-1. Calculated for C₁₀H₁₁N₃O; 189. Observed; 190 (M+H)⁺. ¹H NMR (400 MHz, METHANOL-d4) δ ppm 2.43 (s, 3H) 4.00 (s, 3H) 7.09 (t, J=7.5 Hz, 1H) 7.18 (d, J=8.4 Hz, 1H) 7.48 (t, J=7.4 Hz, 1H) 8.01 (s, 1H).

### EXAMPLE 45

### 3-METHYL-5-(2-PHENOXYPHENYL)-4H-[1,2,4]TRIAZOLE

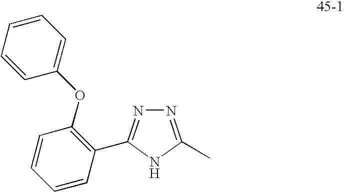

Compound 45-1 was prepared from the appropriate hydrazide in a manner analogous to that described for compound 43-1. Calculated for C₁₅H₁₃N₃O; 251. Observed; 252 (M+H)⁺. ¹H NMR (400 MHz, METHANOL-d4) δ ppm 2.42 (s, 3H) 6.91 (d, J=8.2 Hz, 1H) 7.02 (d, J=7.6 Hz, 2H) 7.13 (t, J=7.0 Hz, 1H) 7.23 (t, J=7.5 Hz, 1H) 7.34 (t, J=7.6 Hz, 2H) 7.41 (t, J=7.3 Hz, 1H) 7.97 (d, J=6.9 Hz, 1H).

### EXAMPLE 46

### 5-(4-TERT-BUTYLPHENYL)-2-METHYL-2H-[1,2,4]TRIAZOLE-3-THIOL

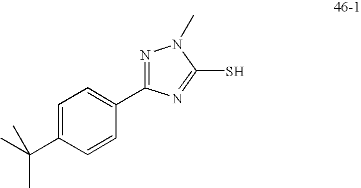

To a solution of 2-methyl-3-thiosemicarbazide (1.06 g, 10.1 mmol) in pyridine (10 mL) was added 4-tert-butylbenzoyl chloride (2 g, 10.1 mmol). The reaction was stirred for 16 hours at room temperature. Aqueous sodium bicarbonate (1 M, 20 mL) was added, and the reaction was heated to reflux for 60 hours. The solution was cooled to room temperature and the title compound was isolated by filtration. Calculated for C₁₃H₁₇N₃S; 247. Observed; 248 (M+H)⁺. ¹H NMR (400 MHz, CHLOROFORM-d) δ ppm 1.34 (s, 9H) 3.86 (s, 3H) 7.51 (d, J=8.6 Hz, 2H) 7.78 (d, J=8.6 Hz, 2H).

### EXAMPLE 47

### 3-(4-TERT-BUTYLPHENYL)-5-METHOXY-4H-[1,2,4]TRIAZOLE

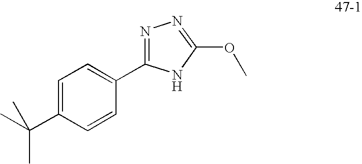

To a solution of 5-(4-tert-Butylphenyl)-[1,3,4]oxadiazol-2-ylamine (1 g, 4.6 mmol) in methanol (50 mL) was added potassium hydroxide (1.27 g, 23 mmol). The reaction was heated to reflux for 3 hours. The solution was cooled to room temperature and concentrated to dryness under vacuum. The title compound was isolated following purification by silica gel chromatography (0-10% methanol in dichloromethane, linear gradient). Calculated for C₁₃H₁₇N₃O; 231. Observed; 232 (M+H)⁺. ¹H NMR (400 MHz, DMSO-d₆) δ ppm 1.25-1.38 (m, 9H) 3.84-4.11 (m, 3H) 7.17-7.66 (m, 2H) 7.71-8.09 (m, 2H) 12.88-13.71 (m, 1H).

### EXAMPLE 48

### 5-(4-TERT-BUTYLPHENYL)-4H-[1,2,4]TRIAZOLE-3-OL

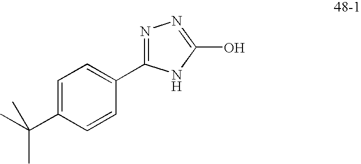

A 50 mL RB flask was charged with 3-(4-tert-Butylphenyl)-5-methoxy-4H-[1,2,4]triazole (150 mg, 0.65 mmol) and concentrated hydrochloric acid (10 mL). The reaction was heated to reflux for 3 hours. After cooling to room temperature, the solution was concentrated to dryness under vacuum. The title compound was isolated following recrystallization from ethanol. Calculated for C₁₂H₁₅N₃O; 217. Observed; 218 (M+H)⁺. ¹H NMR (400 MHz, DMSO-d₆) δ ppm 1.29 (s, 9H) 7.49 (d, J=8.5 Hz, 2H) 7.70 (d, J=8.5 Hz, 2H) 11.94 (s, 1H).

### EXAMPLE 49

### 3-(4-TERT-BUTYLPHENYL)-5-METHYLSULFANYL-4H-[1,2,4]TRIAZOLE

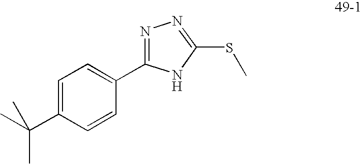

To a solution of 5-(4-tert-butylphenyl)-1H-[1,2,4]triazole-3-thiol (540 mg, 2.3 mmol) in THF (30 mL) was sequentially added aqueous sodium hydroxide (1 M, 7 mL) and iodomethane (137 μL, 2.78 mmol). The mixture was stirred for 2 hours at room temperature. The reaction was quenched by dropwise addition of aqueous hydrochloric acid (1 M). The solution was concentrated to dryness under vacuum. The title compound was isolated following silica gel chromatography (0-100% ethyl acetate in heptane, linear gradient). Calculated for C₁₃H₁₇N₃S; 247. Observed; 248 (M+H)⁺. ¹H NMR (400 MHz, CHLOROFORM-d) δ ppm 1.36 (s, 9H) 2.70 (s, 3H) 7.49 (d, J=8.4 Hz, 2H) 7.89 (d, J=8.6 Hz, 2H).

### EXAMPLE 50

### 5-(4-TERT-BUTYLPHENYL)-4H-[1,2,4]TRIAZOLE-3-YLAMINE

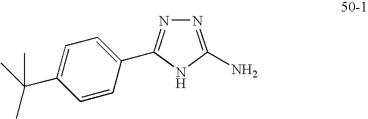

To a solution of aminoguanidine nitrate (2.16 g, 15.8 mmol) in pyridine (52 mL) at 0° C. was slowly added 4-tert-butylbenzoyl chloride (3.26 g, 16.6 mmol). The reaction was allowed to stir for 16 hours at room temperature. Aqueous sodium hydroxide (1 M, 100 mL) was added, and the reaction was heated to reflux for 16 hours. After cooling to room temperature, the solution was acidified by dropwise addition of aqueous hydrochloric acid. The resultant solid was isolated by filtration. The title compound was isolated following purification by silica gel chromatography (0-10% methanol in dichloromethane, linear gradient). Calculated for C₁₂H₁₆N₄; 216. Observed; 217 (M+H)⁺. ¹H NMR (400 MHz, METHANOL-d4) δ ppm 1.33 (s, 9H) 7.46 (s, 1H) 7.79 (d, 2H).

### EXAMPLE 51

### 5-(4-TERT-BUTYLPHENYL)-1-METHYL-1H-[1,2,4]TRIAZOLE-3-THIOL

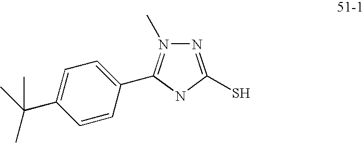

Potassium thiocyanate (371 mg, 3.82 mmol) was dissolved in a minimal amount of ethanol. Aqueous hydrochloric acid (1 M, 25 mL) was added, and the solution stirred for 10 minutes at room temperature. The mixture was added to a solution of 1-(tert-butylbenzoyl)-1-methylhydrazine (393 mg, 1.91 mmol) in ethanol (75 mL). The reaction was heated for 4 hours, and then allowed to stir at room temperature for 60 hours. The solution was concentrated to dryness under vacuum, and the resultant mixture was purified by silica gel chromatography (0-10% methanol in dichloromethane, linear gradient). The isolated product was dissolved in aqueous sodium hydroxide (1 M, 100 mL) and heated to reflux for 16 hours. The solution was cooled to room temperature and acidified by dropwise addition of aqueous hydrochloric acid. The precipitate was isolated by filtration and purified by preparative HPLC to give the title compound. Calculated for C₁₃H₁₇N₃S; 247. Observed; 248 (M+H)⁺. ¹H NMR (400 MHz, METHANOL-d4) δ ppm 1.37 (s, 9H) 3.85 (s, 3H) 7.65 (d, 2H) 7.67 (d, 2H).

### EXAMPLE 52

### 3-(4-TERT-BUTYLPHENYL)-4H-[1,2,4]TRIAZOLE

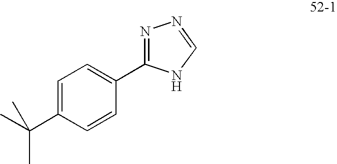

To a solution of 4-tert-butylbenzhydrazide (2 g, 10.4 mmol) in acetonitrile (150 mL) was added dimethylformamide dimethylacetal (1.38 mL, 10.4 mmol). The reaction was heated to 50° C. for 1 hour. 4-Fluorobenzylamine (1.07 mL, 9.45 mmol) was added, followed by acetic acid (7 mL). The solution was heated to 120° C. and stirred for 16 hours. The reaction was cooled to room temperature and concentrated to dryness under vacuum. The mixture was purified by silica gel chromatography (0-5% methanol in ethyl acetate) to give 3-(4-tert-Butylphenyl)-4-(4-fluorobenzyl)-4H-[1,2,4]triazole as a white solid.

The solid was dissolved in ethanol (150 mL) and palladium (II) hydroxide (10% on carbon, 100 mg) was added. The solution was degassed and fitted with a hydrogen balloon. The reaction stirred for 16 hours at room temperature. The reaction was again degassed, and filtered through Celite to remove the palladium catalyst. The filtrate was concentrated to dryness under vacuum and purified by silica gel chromatography (0-5% methanol in ethyl acetate) to yield the title compound. Calculated for C₁₂H₁₅N₃; 201. Observed; 202 (M+H)⁺. ¹H NMR (400 MHz, CHLOROFORM-d) δ ppm 1.35 (s, 9H) 7.50 (d, J=8.25 Hz, 2H) 7.94 (d, J=8.15 Hz, 2H) 8.19 (br. s., 1H).

### EXAMPLE 53

### 3-(4-TERT-BUTYLPHENYL)-5-METHYL-4H-[1,2,4]TRIAZOLE

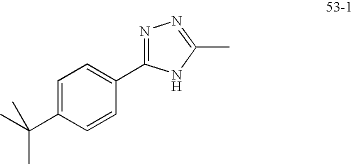

Compound 53-1 was prepared from the appropriate hydrazide and amide dimethylacetal in a manner analogous to that described for compound 52-1. Calculated for C₁₃H₁₇N₃; 215. Observed; 216 (M+H)⁺. ¹H NMR (400 MHz, CHLOROFORM-d) δ ppm 1.34 (s, 9H) 3.86 (s, 3H) 7.51 (d, J=8.6 Hz, 2H) 7.78 (d, J=8.6 Hz, 2H).

### EXAMPLE 54

### 3-METHYL-5-(4-PENTYLPHENYL)-4H-[1,2,4]TRIAZOLE

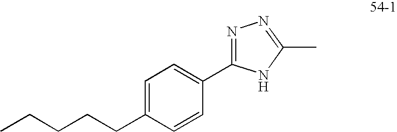

Compound 54-1 was prepared from the appropriate hydrazide and amide dimethylacetal in a manner analogous to that described for compound 52-1. Calculated for C₁₄H₁₉N₃; 229. Observed; 230 (M+H)⁺. ¹H NMR (400 MHz, CHLOROFORM-d) δ ppm 2.11-2.28 (m, J=14.3, 7.1, 7.1 Hz, 2H) 3.16 (t, J=7.0 Hz, 2H) 3.34 (t, J=7.1 Hz, 2H) 3.58 (s, 3H) 7.40-7.51 (m, J=7.6, 7.6 Hz, 2H) 7.51-7.60 (m, 1H) 7.90-8.00 (m, 2H) 8.13 (s, 1H).

### EXAMPLE 55

### [5-(4-TERT-BUTYLPHENYL)-4-(4-FLUOROBENZYL)-4H-[1,2,4]TRIAZOLE-3-YL]-METHANOL

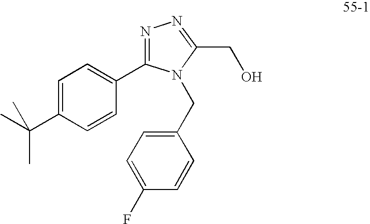

To a solution of 3-(4-tert-Butylphenyl)-4-(4-fluorobenzyl)-4H-[1,2,4]triazole (0.47 g, 1.52 mmol) in dry THF (30 mL) at −78° C. was added n-butyllithium (1.6 M in hexane, 1.14 mL, 1.82 mmol). The reaction was stirred for 45 minutes, and then DMF (0.47 mL, 6.08 mmol) was added dropwise. The solution was stirred for an additional 4 hours at −78° C. The reaction was warmed to room temperature and quenched by dropwise addition of saturated aqueous ammonium chloride. The mixture was partitioned between ethyl acetate (50 mL) and water (30 mL). The organic portion was washed with brine, dried over sodium sulfate, and concentrated to dryness under vacuum. 5-(4-tert-Butylphenyl)-4-(4-fluorobenzyl)-4H-[1,2,4]triazole-3-carbaldehyde was isolated as a white solid.

The crude product was redissolved in methanol (10 mL). Water (5 mL) and sodium borohydride (227 mg, 6 mmol) were added, and the reaction stirred for 5 hours at room temperature. The solution was concentrated to dryness and purified by preparative HPLC, yielding the title compound. Calculated for C₂₀H₂₂FN₃O 339; Observed; 340 (M+H)⁺; ¹H NMR (400 MHz, METHANOL-d4) δ ppm 1.33 (s, 9H) 4.71 (s, 2H) 5.41 (s, 2H) 7.42 (d, J=8.4 Hz, 2H) 7.52 (d, J=8.4 Hz, 2H).

### EXAMPLE 56

### [5-(4-TERT-BUTYLPHENYL)-4H-[1,2,4]TRIAZOLE-3-YL]-METHANOL

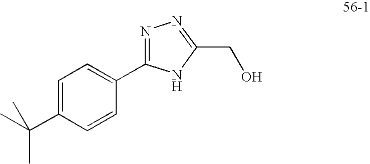

A solution of [5-(4-tert-Butylphenyl)-4-(4-fluorobenzyl)-4H-[1,2,4]triazol-3-yl]-methanol (0.2 g, 0.59 mmol) in methanol (50 mL) was degassed and purged with nitrogen. Palladium hydroxide (10% on carbon, 100 mg) was added, the reaction was fitted with a hydrogen balloon, and the mixture was allowed to stir at room temperature for 16 hours. The reaction was degassed and the catalyst was removed by filtration through Celite. The filtrate was concentrated to dryness under vacuum and purified by preparative HPLC to yield the title compound. Calculated for C₁₃H₁₇N₃O; 231. Observed; 232 (M+H)⁺. ¹H NMR (400 MHz, METHANOL-d4) δ ppm 1.32 (s, 9H) 4.71 (s, 2H) 7.49 (d, J=8.5 Hz, 2H) 7.87 (d, J=8.5 Hz, 2H).

### EXAMPLE 57

### 3-(4-TERT-BUTYLPHENYL)-5-DIFLUOROMETHYL-4H-[1,2,4]TRIAZOLE

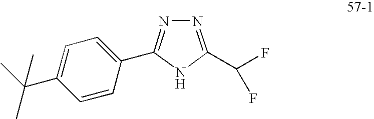

To a solution of 5-(4-tert-Butylphenyl)-4-(4-fluorobenzyl)-4H-[1,2,4]triazole-3-carbaldehyde (0.25 g, 0.74 mmol) in dichloromethane (20 mL) was added bis(2-methoxyethyl)amino-sulfur trifluoride (0.65 g, 2.96 mmol). The reaction was heated to reflux for 90 minutes. The solution was cooled to room temperature and concentrated to dryness under vacuum. The crude product was taken up in ethanol (100 mL) and palladium (II) hydroxide (10% on carbon, 100 mg) was added. The reaction was degassed and fitted with a hydrogen balloon. The mixture was allowed to stir at room temperature for 16 h. The reaction was again degassed and the catalyst was removed by filtration through Celite. The resultant mixture was purified by silica gel chromatography (0-5% methanol in ethyl acetate) to generate the title compound. Calculated for C₁₃H₁₅F₂N₃; 251. Observed; 252 (M+H)⁺. ¹H NMR (400 MHz, CHLOROFORM-d) δ ppm 1.35 (s, 9H) 6.78 (t, J=53.6 Hz, 1H) 7.52 (d, J=8.5 Hz, 2H) 7.85 (d, J=8.5 Hz, 2H).

### EXAMPLE 58

### 5-(4-TERT-BUTYLPHENYL)-[1,3,4]OXADIAZOL-2-YLAMINE

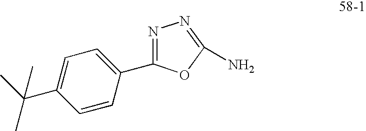

To a solution of tert-butylbenzhydrazide (4 g, 20.8 mmol) in 1,4-dioxane (70 mL) was added cyanogen bromide (2.64 g, 24.9 mmol). A solution of sodium bicarbonate (1.76 g) in water (50 mL) was added slowly, resulting in significant gas evolution. The reaction was stirred for 1 hour at room temperature, and then diluted with 9:1 dichloromethane/methanol (100 mL). The organic portion was washed with brine, dried over sodium sulfate, and concentrated to dryness under vacuum. The title compound was isolated after recrystallization from ethyl acetate. Calculated for C₁₂H₁₅N₃O; 217. Observed; 218 (M+H)⁺. ¹H NMR (400 MHz, DMSO-d₆) δ ppm 1.30 (s, 9H) 7.19 (s, 2H) 7.55 (d, J=8.6 Hz, 2H) 7.73 (d, J=8.6 Hz, 2H).

### EXAMPLE 59

### 5-(4-TERT-BUTYLPHENYL)-[1,3,4]OXADIAZOLE-2-THIOL

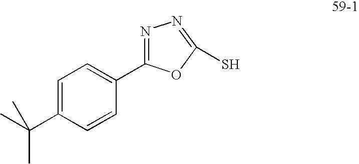

To a solution of tert-butylbenzhydrazide (2 g, 10.4 mmol) in ethanol (30 mL) was added carbon disulfide (1.97 g, 26 mmol) and potassium hydroxide (0.58 g, 26 mmol). The reaction was heated to reflux for 16 hours. The solution was concentrated to dryness under vacuum, and the resultant mixture was purified by silica gel chromatography (0-10% methanol in dichloromethane). Calculated for C₁₂H₁₄N₂OS; 234. Observed; 235 (M+H)⁺. ¹H NMR (400 MHz, DMSO-d₆) δ ppm 1.31 (s, 9H) 7.61 (d, J=8.6 Hz, 2H) 7.81 (d, J=8.6 Hz, 2H).

### EXAMPLE 60

### 2-(4-TERT-BUTYLPHENYL)-5-METHYL-[1,3,4]OXADIAZOLE

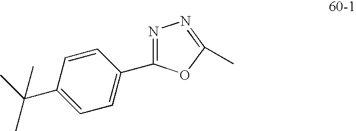

To a solution of tert-butylbenzhydrazide (0.2 g, 1.04 mmol) in acetonitrile (2 mL) was added dimethylacetamide dimethylacetal (0.15 mL, 1.04 mmol). The reaction was heated to 80° C. for 1 hour. Acetic acid (1 mL) was added, and the reaction was refluxed for an additional hour. The reaction was cooled to room temperature and concentrated to dryness under vacuum. The title compound was isolated following purification by preparative HPLC. Calculated for C₁₃H₁₆N₂O; 216. Observed; 217 (M+H)⁺. ¹H NMR (400 MHz, CHLOROFORM-d) δ ppm 1.36 (s, 9H) 2.62 (s, 3H) 7.52 (d, J=8.6 Hz, 2H) 7.96 (d, J=8.6 Hz, 2H).

### EXAMPLE 61

### 3-(4-TERT-BUTYLPHENYL)-5-METHYL-[1,2,4]OXADIAZOLE

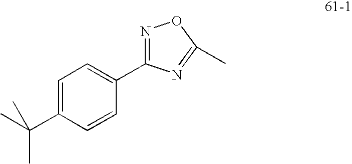

To a solution of 4-tert-butylbenzonitrile (2 g, 12.6 mmol) in methanol (50 mL) was added aqueous hydroxylamine (50 mL), and the reaction was heated to reflux for 16 hours. The solution was cooled to room temperature and concentrated to dryness under vacuum. The resultant solid was redissolved in pyridine (40 mL) and cooled to −78° C. Acetyl chloride (7 mL, 9.8 mmol) was added, and the solution was heated to reflux for 16 hours. The reaction was cooled to room temperature and concentrated to dryness under vacuum. The resultant mixture was diluted with ethyl acetate and washed with aqueous hydrochloric acid (2 M). The title compound was isolated following purification by silica gel chromatography. Calculated for C₁₃H₁₆N₂O; 216. Observed; 217 (M+H)⁺. ¹H NMR (400 MHz, CHLOROFORM-d) δ ppm 1.34 (s, 9H) 2.64 (s, 3H) 7.49 (d, J=8.5 Hz, 2H) 7.98 (d, J=8.5 Hz, 2H).

### EXAMPLE 62

### 5-(4-TERT-BUTYLPHENYL)-3-METHYL-[1,2,4]OXADIAZOLE

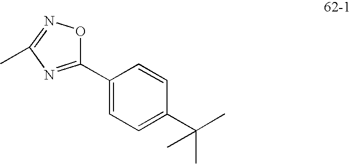

A mixture of acetonitrile (3.93 g, 95.8 mmol) and saturated aqueous hydroxylamine (5 mL) was heated to reflux for 16 hours. The solution was cooled to room temperature and concentrated to dryness under vacuum. The resultant solid product was redissolved in pyridine (20 mL), and 4-tert-butylbenzoyl chloride (9.3 g, 48 mmol) was added. The reaction was heated to reflux for 16 hours. The reaction was cooled to room temperature, concentrated to dryness under vacuum, and diluted with ethyl acetate (200 mL). The solution was washed with aqueous hydrochloric acid (10%). The organic layer was dried over sodium sulfate and concentrated under vacuum. The title compound was isolated following purification by preparative HPLC. Calculated for C₁₃H₁₆N₂O; 216. Observed; 217 (M+H)⁺. ¹H NMR (400 MHz, CHLOROFORM-d) δ ppm 1.35 (s, 9H) 2.46 (s, 3H) 7.53 (d, J=8.5 Hz, 2H) 8.03 (d, J=8.5 Hz, 2H).

### EXAMPLE 63

### 5-(4-TERT-BUTYLPHENYL)-2-METHYL-1H-IMIDAZOLE

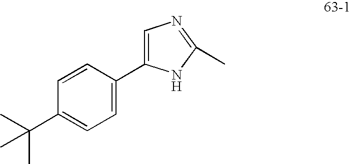

To a solution of acetamidine hydrochloride (0.094 g, 1 mmol) in DMF (15 mL) was added 4-tert-butylphenacyl chloride (0.21 g, 1 mmol) and potassium carbonate (1.38 g, 10 mmol). The reaction was heated to reflux for 90 minutes. The solution was cooled to room temperature and filtered to remove excess potassium carbonate. The filtrate was concentrated to dryness under vacuum, and the resultant solid was purified by preparative HPLC to yield the title compound. Calculated for C₁₄H₁₈N₂; 214. Observed; 215 (M+H)⁺. ¹H NMR (400 MHz, CHLOROFORM-d) δ ppm 1.33 (s, 9H) 2.46 (s, 3H) 7.17 (s, 1H) 7.39 (d, J=8.5 Hz, 2H) 7.60 (d, J=8.5 Hz, 2H).

### EXAMPLE 64

### 2-(4-TERT-BUTYLPHENYL)-5-METHYL-1H-IMIDAZOLE

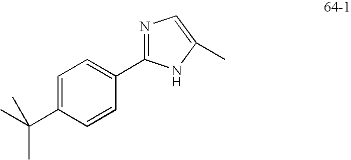

To a solution of 4-tert-butylbenzamidine (0.176 g, 1 mmol) in THF (8 mL) was added potassium bicarbonate (0.2 g, 2 mmol) in water (2 mL). Chloroacetone (0.092 g, 1 mmol) in THF (2 mL) was added dropwise over several minutes, and the reaction was then heated to reflux for 4 hours. The solution was concentrated to dryness under vacuum, and the resultant solid was purified by preparative HPLC to yield the title compound. Calculated for C₁₄H₁₈N₂; 214. Observed; 215 (M+H)⁺. ¹H NMR (400 MHz, METHANOL-d4) δ ppm 1.33 (s, 9H) 2.26 (d, J=0.7 Hz, 3H) 6.77 (s, 1H) 7.46 (d, J=8.5 Hz, 2H) 7.73 (d, J=8.5 Hz, 2H).

### EXAMPLE 65

### 2-(5-METHYL-4H-[1,2,4]TRIAZOL-3-YL)-PYRIDINE

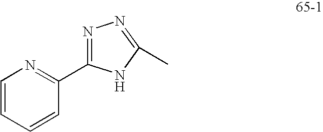

Calculated for C₈H₈N₄; 160. Observed; 161 (M+H)⁺. ¹H NMR (400 MHz, CHLOROFORM-d) δ ppm 2.54 (s, 3H) 7.30-7.50 (m, 1H) 7.87 (t, J=7.43 Hz, 1H) 8.20 (d, J=7.74 Hz, 1H) 8.70 (s, 1H).

### EXAMPLE 66

### 3-(5-METHYL-4H-[1,2,4]TRIAZOL-3-YL)-PYRIDINE

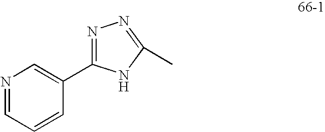

Calculated for C₈H₈N₄; 160. Observed; 161 (M+H)⁺. ¹H NMR (400 MHz, CHLOROFORM-d) δ ppm 2.56 (s, 3H) 7.41 (dd, J=7.7, 5.0 Hz, 1H) 8.38 (d, J=7.9 Hz, 1H) 8.65 (d, J=3.9 Hz, 1H) 9.34 (s, 1H).

### EXAMPLE 67

### 4-(5-METHYL-4H-[1,2,4]TRIAZOL-3-YL)-PYRIDINE

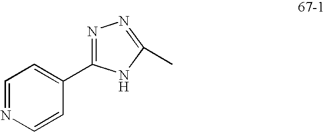

Calculated for C₈H₈N₄; 160. Observed; 161 (M+H)⁺. ¹H NMR (400 MHz, CHLOROFORM-d) δ ppm 2.59 (s, 3H) 8.00 (d, J=5.7 Hz, 2H) 8.71 (d, J=5.6 Hz, 2H).

### EXAMPLE 68

### 3-METHYL-5-THIOPHEN-2-YL-4H-[1,2,4]TRIAZOLE

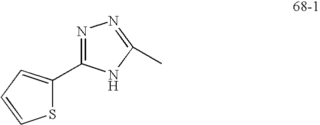

Calculated for C₇H₇N₃S; 165. Observed; 166 (M+H)⁺. ¹H NMR (400 MHz, METHANOL-d4) δ ppm 0.92 (s, 3H) 5.48-5.68 (m, 1H) 5.95 (s, 1H) 6.08 (d, J=3.2 Hz, 1H).

### EXAMPLE 69

### Ncad-Fc Bead-Bead Aggregation Assay

Compounds of the invention were assayed according to the following procedures.

a. Preparation of Ncad-Fc Beads

10 μl of magnetic Dynabeads® Protein A (Prod. No. 100.01) were pipetted into a 1.5 ml eppendorf tube and washed with 0.5 ml of PBS (1×), 0.1% Tween 20, and 2 mM EGTA using the Dynal MPC-S Magnetic Particle Concentrator. Supernatant was aspirated, the magnet was removed and beads were washed once more with same buffer using the Dynal MPC-S Magnetic Particle Concentrator. Following this supernatant was once again removed and the beads were resuspended in 10 μl of PBS (1×), 0.1% Tween 20, and 2 mM EGTA buffer.

An equal 10 ul volume of Ncad-Fc protein (0.25 μg/10 μl, chicken N-cadherin ectodomain fused to the Fc fragment of mouse IgG2b) or human Fc (Jackson Immunoresearch, concentration 2.3 mg/ml) suspended in PBS (1×), 0.1% Tween 20, 2 mM EGTA and 1% BSA (Sigma, Prod. No. A0281) was added and incubated for 1-2 hours at room temperature on the Vortex Genie 2, followed by three washes in 0.5 ml of the same buffer. The beads were then resuspended in 80 μl PBS (1×) plus 1% BSA (dilution 1/8) and kept on ice.

b. Bead-Bead Aggregation

The aggregation assay was performed in duplicates in 12 well/6 mm slides (CEL-LINE/ERIE SCIENTIFIC CO. Prod. No. 10-103). Experimentation was conducted in eppendorf tubes containing 200 μl of DMEM medium (1000 mg/ml glucose, Gibco Prod. No. 21 885-025)+10% Fetal Calf Serum (FCS) containing an N-cadherin inhibitor or calcium chelator (EDTA, EGTA) with the addition of 4 μl of N-cadherin coated Dynabeads®. The resulting bead solutions were gently mixed on ice followed by 50 μl of solution deposited per well.

Slides were incubated for 30 minutes to 1 hour at 37° C. and 5% CO₂. After incubation two images per well were recorded on an inverted microscope (Nikon Diaphot) at 20× magnification, with an 8 Volt illumination using a Nikon D100. Images were analyzed using the Bead Counting software program from Metamorph (Meta Imaging Series 6.2r6).

In this assay, in the absence of inhibitor, the beads bind to each other and aggregate due to dimerization of the N-cadherin molecules on the surfaces of the beads. Accordingly, compounds effective for disrupting N-cadherin-mediated cell adhesion can be identified on the basis of whether they disrupt bead-bead aggregation in this assay.

Using this approach, illustrative compounds of the invention were tested and the following representative compounds were determined to be active: compound 1-1, compound 5-1, compound 7-1, compound 8-1, compound 9-1, compound 10-1, compound 11-1, compound 12-1, compound 14-1, compound 50-1, compound 53-1, compound 65-1, compound 66-1, compound 67-1, and compound 68-1.

In addition, the following compounds were also determined to be active in this assay.

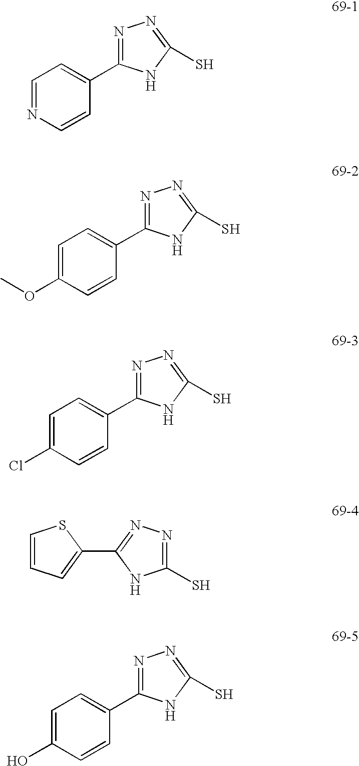

c. Culture of Retinal Explants and Quantification of Neurite Outgrowth:

Tissue culture dishes were coated with nitrocellulose and allowed to dry (Lagenaur and Lemmon, [citation?] 1987). Substrate protein (Human N-cadherin-Fc or Laminin) was spread across the central region of each dish. Retinal explant cultures were made according to a previously described procedure (Halfter, W. et al., Dev. Biol., 95:56-64, 1983; Drazba, J. and Lemmon, V., Dev. Biol., 138:82-93, 1990). In brief, embryonic day 8 (stage 32-34 according to Hamburger and Hamilton, [citation?] 1951) White Leghorn chick eyes were dissected and the retina was flattened with the photoreceptor side down onto black nitrocellulose filters that had previously been incubated in concanavalin A. The filter was then cut into strips perpendicular to the optic fissure. Strips were inverted onto substrate-coated culture dishes so that the gang lion cell layer was directly adjacent to the substratum. N-cadherin small molecule antagonists were diluted in the culture medium and added at the time of plating. Neurite outgrowth was examined at approximately 20 hours after plating.

Neurite outgrowth from retina explants was catalogued using a SPOT RT digital camera and image acquisition software. The length of the five longest neurites per explant were measured (Burden-Gulley, S. M. and Brady-Kalnay, S. M. J. Cell Biol., 144:1323-1336, 1999). To measure neurite density, the region of neurite outgrowth was outlined to define the region of interest and the neurites were highlighted using Metamorph software. Data from similar experimental conditions were combined, analyzed by Student's t test and plotted.

Using this approach, illustrative compounds of the invention were tested and the following representative compounds were confirmed to inhibit neurite outgrowth: compound 46-1, compound 57-1, compound 69-2, compound 69-4, compound 1-1, compound 28-1, compound 53-1, compound 21-1, compound 22-1, compound 25-1, compound 26-1, compound 64-1.

In addition, the following compounds were also determined to be active in this assay:

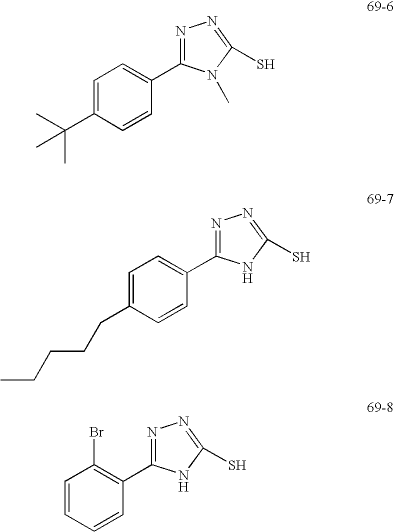

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

We claim:

1. A compound having the following structure (I): 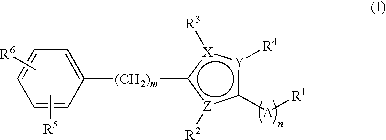
or a pharmaceutically acceptable salt, stereoisomer or prodrug thereof, wherein
A is —NH—, —O— or —S—;
X and Y are independently nitrogen, oxygen or carbon;
Z is nitrogen or oxygen;
R¹ is hydrogen, optionally substituted alkyl, optionally substituted aryl or optionally substituted heterocycle;
R², R³ and R⁴ are independently either present or absent and when present are independently hydrogen, optionally substituted alkyl, optionally substituted aryl or optionally substituted heterocycle, except that R², R³ and R⁴ cannot be carboxyl;
R⁵ and R⁶ are independently hydrogen, halogen, optionally substituted alkyl, optionally substituted aryl, optionally substituted heterocycle or —OR⁷, or R⁵ and R⁶, when attached to adjacent carbons of the phenyl ring, join to form an optionally substituted, fused aryl group;
R⁷ is hydrogen, lower alkyl, aryl or alkylaryl;
m and n are independently 0 or 1; and
the ring formed by X, Y and Z is aromatic.

2. The compound of claim 1 wherein A is —S—, X, Y and Z are nitrogen, m is 0 and n is 1, and the compound has the following structure (II): 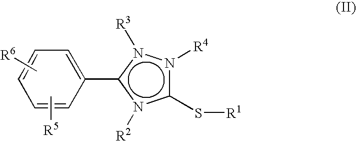
or a pharmaceutically acceptable salt, stereoisomer or prodrug thereof.

3. The compound of claim 2 wherein R¹ is hydrogen or methyl, at least two of R², R³ and R⁴ are absent and at least one of R², R³ and R⁴ is hydrogen, methyl or ethyl.

4. The compound of claim 1 wherein A is —O—, X, Y and Z are nitrogen, m is 0 and n is 1, and the compound has the following structure (III): 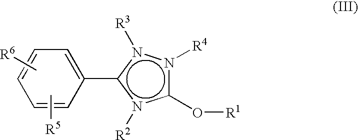
or a pharmaceutically acceptable salt, stereoisomer or prodrug thereof.

5. The compound of claim 4 wherein R¹ is hydrogen or methyl, at least two of R², R³ and R⁴ are absent and at least one of R², R³ and R⁴ is hydrogen.

6. The compound of claim 1 wherein A is —NH—, X, Y and Z are nitrogen, m is 0 and n is 1, and the compound has the following structure (IV): 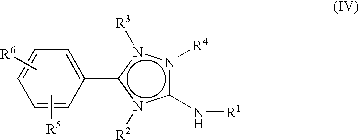
or a pharmaceutically acceptable salt, stereoisomer or prodrug thereof.

7. The compound of claim 6 wherein R¹ is hydrogen, at least two of R², R³ and R⁴ are absent and at least one of R², R³ and R⁴ is hydrogen.

8. The compound of claim 1 wherein A is —S—, X and Y are nitrogen, Z is oxygen, m is 0 and n is 1, and the compound has the following structure (V): 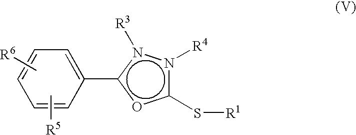
or a pharmaceutically acceptable salt, stereoisomer or prodrug thereof.

9. The compound of claim 8 wherein R¹ is hydrogen and R³ and R⁴ are absent.

10. The compound of claim 1 wherein A is —NH—, X and Y are nitrogen, Z is oxygen, m is 0 and n is 1, and the compound has the following structure (VI): 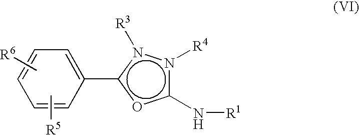
or a pharmaceutically acceptable salt, stereoisomer or prodrug thereof.

11. The compound of claim 10 wherein R¹ is hydrogen and R³ and R⁴ are absent.

12. The compound of claim 1 wherein X, Y and Z are nitrogen and m and n are 0, and the compound has the following structure (VII): 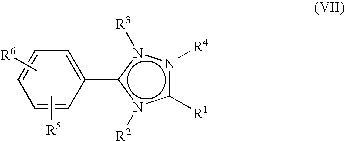
or a pharmaceutically acceptable salt, stereoisomer or prodrug thereof.

13. The compound of claim 12 wherein R¹ is hydrogen or optionally substituted alkyl, at least two of R², R³ and R⁴ are absent and at least one of R², R³ and R⁴ is hydrogen.

14. The compound of claim 12 wherein R¹ is methyl, at least two of R², R³ and R⁴ are absent and at least one of R², R³ and R⁴ is hydrogen.

15. The compound of claim 12 wherein R¹ is optionally substituted aryl, at least two of R², R³ and R⁴ are absent and at least one of R², R³ and R⁴ is hydrogen.

16. The compound of claim 12 wherein R¹ is optionally substituted heterocycle, at least two of R², R³ and R⁴ are absent and at least one of R², R³ and R⁴ is hydrogen.

17. The compound of claim 1 wherein X and Y are nitrogen, Z is oxygen and m and n are 0, and the compound has the following structure (VIII): 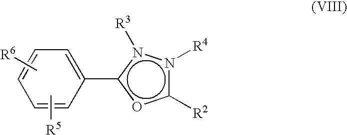
or a pharmaceutically acceptable salt, stereoisomer or prodrug thereof.

18. The compound of claim 17 wherein R¹ is methyl and R³ and R⁴ are absent.

19. The compound of claim 1 wherein m and n are 0 and either Y and Z are nitrogen, X is oxygen and R³ is absent or X and Z are nitrogen, Y is oxygen and R⁴ is absent, and the compound has one of the following structures (IX) and (X): 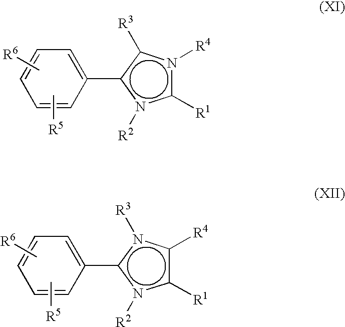
or a pharmaceutically acceptable salt, stereoisomer or prodrug thereof.

20. The compound of claim 19 wherein R¹ is methyl and R², R³ and R⁴ are all absent.

21. The compound of claim 1 wherein m and n are 0 and either Y and Z are nitrogen and X is carbon or X and Z are nitrogen and Y is carbon, and the compound has one of the following structures (X₁) and (XII): 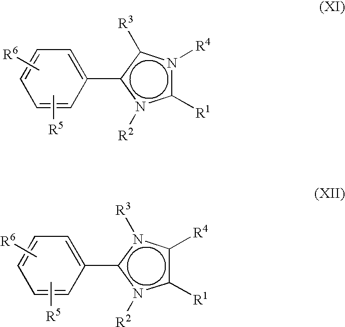
or a pharmaceutically acceptable salt, stereoisomer or prodrug thereof.

22. The compound of claim 21 wherein R¹ is methyl and R² and R³ are hydrogen and R⁴ is absent in structure (XI) or R¹ is methyl and R² and R⁴ are hydrogen and R³ is absent in structure (XII).

23. The compound of claim 1 wherein X, Y and Z are nitrogen, m is 1 and n is 0, and the compound has the following structure (XIII): 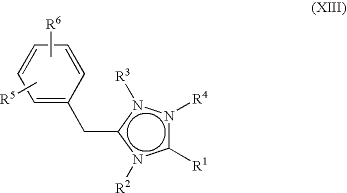
or a pharmaceutically acceptable salt, stereoisomer or prodrug thereof.

24. The compound of claim 23 wherein R¹ is methyl, R² is hydrogen and R³ and R⁴ are absent.

25. The compound of claim 1 wherein at least one of R⁵ and R⁶ has the following structure:

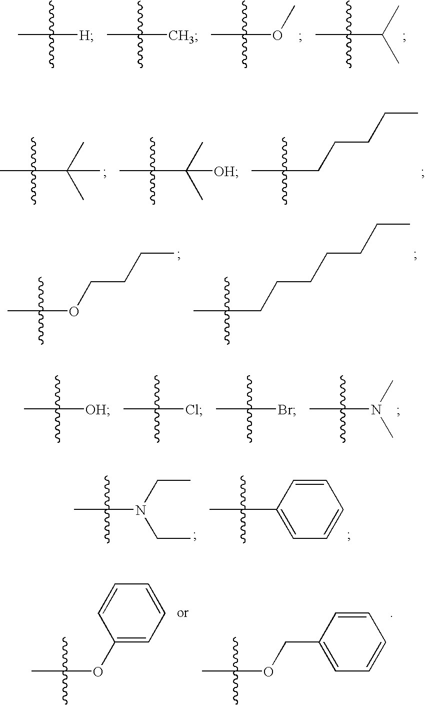

26. The compound of claim 1 wherein R⁵ and R⁶ are attached to adjacent atoms of the phenyl ring and are taken together with the carbon atoms to which they are attached to form an optionally substituted, fused phenyl ring, and the compound has one of the following structures (XIV) and (XV): 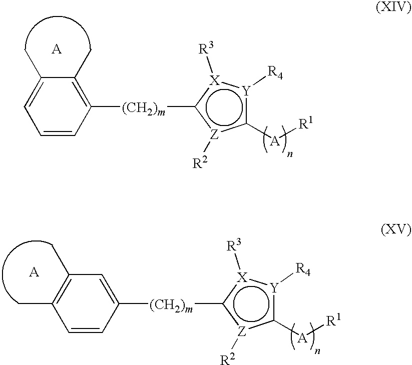
or a pharmaceutically acceptable salt, stereoisomer or prodrug thereof, wherein
A is an optionally substituted, fused phenyl ring.

27. The compound of claim 1 wherein n is 1 and R¹ has the following structure:

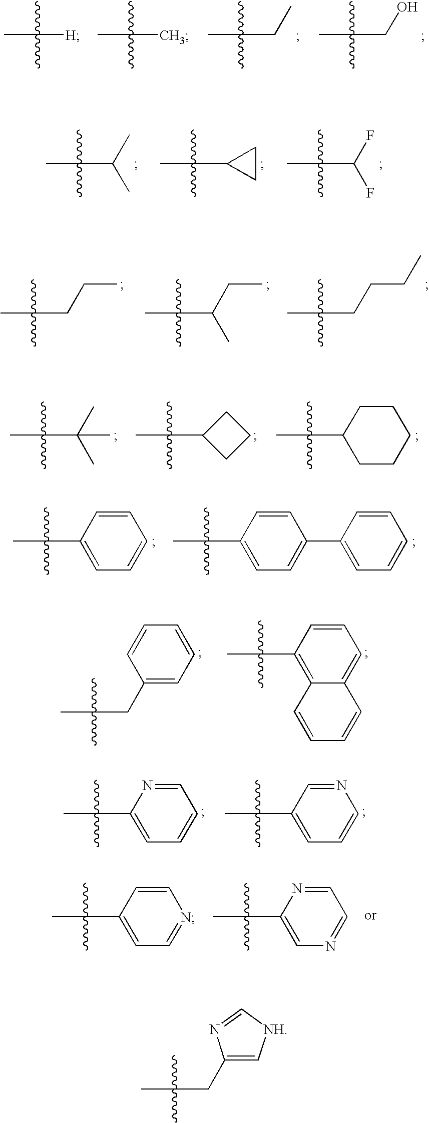

28. The compound of claim 1 wherein Z is nitrogen and R² has the following structure:

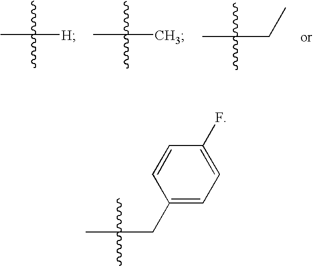

29. The compound of claim 1 wherein the compound is: 3-(4-tert-Butylphenyl)-5-ethyl-4H-[1,2,4]triazole; 3-Methyl-5-naphthalen-2-yl-4H-[1,2,4]triazole; 3-Methyl-5-phenyl-4H-[1,2,4]triazole; 3-Methyl-5-o-tolyl-4H-[1,2,4]triazole; 3-Methyl-5-m-tolyl-4H-[1,2,4]triazole; 3-Methyl-5-p-tolyl-4H-[1,2,4]triazole; 3-(2-Chlorophenyl)-5-methyl-4H-[1,2,4]triazole; 3-(3-Chlorophenyl)-5-methyl-4H-[1,2,4]triazole; 3-(4-Chlorophenyl)-5-methyl-4H-[1,2,4]triazole; 3-Benzyl-5-methyl-4H-[1,2,4]triazole; 3-(3-Methoxyphenyl)-5-methyl-4H-[1,2,4]triazole; 3-(4-Methoxyphenyl)-5-methyl-4H-[1,2,4]triazole; 3-Methyl-5-(4-phenoxyphenyl)-4H-[1,2,4]triazole; 3-(3,4-Dichlorophenyl)-5-methyl-4H-[1,2,4]triazole; 3-Biphenyl-4-yl-5-methyl-4H-[1,2,4]triazole; 3-Methyl-5-(3-phenoxyphenyl)-4H-[1,2,4]triazole; 3-(2,4-Dichlorophenyl)-5-methyl-4H-[1,2,4]triazole; 3-Heptyl-5-methyl-4H-[1,2,4]triazole; 3-(4-tert-Butylphenyl)-5-propyl-4H-[1,2,4]triazole; 3-Butyl-5-(4-tert-butylphenyl)-4H-[1,2,4]triazole; 3-(4-tert-Butylphenyl)-5-isopropyl-4H-[1,2,4]triazole; 3-(4-tert-Butylphenyl)-5-cyclopropyl-4H-[1,2,4]triazole; 3-(4-tert-Butylphenyl)-5-cyclohexyl-4H-[1,2,4]triazole; 3-(4-tert-Butylphenyl)-5-phenyl-4H-[1,2,4]triazole; 3-(4-tert-Butylphenyl)-5-cyclobutyl-4H-[1,2,4]triazole; 3-Benzyl-5-(4-tert-butylphenyl)-4H-[1,2,4]triazole; 4-[5-(4-tert-Butylphenyl)-4H-[1,2,4]triazol-3-yl]-pyridine; 2-[5-(4-tert-Butylphenyl)-4H-[1,2,4]triazol-3-yl]-pyrazine; Dimethyl-[4-(5-methyl-4H-[1,2,4]triazol-3-yl)-phenyl]-amine; 3-(4-Benzyloxyphenyl)-5-methyl-4H-[1,2,4]triazole; 3-(4-Isopropylphenyl)-5-methyl-4H-[1,2,4]triazole; 3-(4-Butoxyphenyl)-5-methyl-4H-[1,2,4]triazole; 2-[5-(4-tert-Butylphenyl)-4H-[1,2,4]triazol-3-yl]-pyridine; 3-[5-(4-tert-Butylphenyl)-4H-[1,2,4]triazol-3-yl]-pyridine; 3-Methyl-5-naphthalen-1-yl-4H-[1,2,4]triazole; 2-[4-(5-Methyl-4H-[1,2,4]triazol-3-yl)-phenyl]-propan-2-ol; 3-sec-Butyl-5-(4-tert-butylphenyl)-4H-[1,2,4]triazole; 3-tert-Butyl-5-(4-tert-butylphenyl)-4H-[1,2,4]triazole; 3-Biphenyl-4-yl-5-(4-tert-butylphenyl)-4H-[1,2,4]triazole; 3-(4-tert-Butylphenyl)-5-naphthalen-1-yl-4H-[1,2,4]triazole; 3-(4-tert-Butylphenyl)-5-(1H-imidazol-4-ylmethyl)-4H-[1,2,4]triazole; Diethyl-[4-(5-methyl-4H-[1,2,4]triazol-3-yl)-phenyl]-amine; 3-Methyl-5-naphthalen-1-ylmethyl-4H-[1,2,4]triazole; 3-(2-Methoxyphenyl)-5-methyl-4H-[1,2,4]triazole; 3-Methyl-5-(2-phenoxyphenyl)-4H-[1,2,4]triazole; 5-(4-tert-Butylphenyl)-2-methyl-2H-[1,2,4]triazole-3-thiol; 3-(4-tert-Butylphenyl)-5-methoxy-4H-[1,2,4]triazole; 5-(4-tert-Butylphenyl)-4H-[1,2,4]triazol-3-ol; 3-(4-tert-Butylphenyl)-5-methylsulfanyl-4H-[1,2,4]triazole; 5-(4-tert-Butylphenyl)-4H-[1,2,4]triazol-3-ylamine; 5-(4-tert-Butylphenyl)-1-methyl-1H-[1,2,4]triazole-3-thiol; 3-(4-tert-Butylphenyl)-4H-[1,2,4]triazole; 3-(4-tert-Butylphenyl)-5-methyl-4H-[1,2,4]triazole; 3-Methyl-5-(4-pentylphenyl)-4H-[1,2,4]triazole; [5-(4-tert-Butylphenyl)-4-(4-fluorobenzyl)-4H-[1,2,4]triazol-3-yl]-methanol; [5-(4-tert-Butylphenyl)-4H-[1,2,4]triazol-3-yl]-methanol; 3-(4-tert-Butylphenyl)-5-difluoromethyl-4H-[1,2,4]triazole; 5-(4-tert-Butylphenyl)-[1,3,4]oxadiazol-2-ylamine; 5-(4-tert-Butylphenyl)-[1,3,4]oxadiazole-2-thiol; 2-(4-tert-Butylphenyl)-5-methyl-[1,3,4]oxadiazole; 3-(4-tert-Butylphenyl)-5-methyl-[1,2,4]oxadiazole; 5-(4-tert-Butylphenyl)-3-methyl-[1,2,4]oxadiazole; 5-(4-tert-Butylphenyl)-2-methyl-1H-imidazole or 2-(4-tert-Butylphenyl)-5-methyl-1H-imidazole.

30. A cell adhesion modulating composition comprising a compound of claim 1 in combination with a pharmaceutically acceptable carrier or diluent.

31. A method for inhibiting cadherin-mediated cell adhesion in a subject comprising the step of administering to a subject in need of such treatment a therapeutically effective amount of a composition of claim 30.

32. The method of claim 31 wherein the method provides for reducing unwanted cellular adhesion in a mammal.

33. The method of claim 31 wherein the method provides for inhibiting the development of cancer in a mammal.

34. The method of claim 31 wherein the method provides for inhibiting angiogenesis in a mammal.

35. The method of claim 31 wherein the method provides for increasing vasopermeability in a mammal.

36. The method of claim 31 wherein the method provides for inhibiting neurite outgrowth.

37. The method of claim 31 wherein the method provides for enhancing apoptosis.

38. A method for inhibiting classical cadherin-mediated intercellular adhesion, comprising contacting a classical cadherin-expressing cell with a composition of claim 30.
